(19) United States
(12) Patent Application Publication
(10) Pub. No.: US 2024/0352438 A1
(43) Pub. Date: Oct. 24, 2024

(54) MEANS AND METHODS FOR DETOXIFYING OCHRATOXIN A

(71) Applicant: DSM Austria GmbH, Getzersdorf (AT)

(72) Inventors: Shreenath PRASAD, Getzersdorf (AT); Christoph GONAUS, Getzersdorf (AT); Wulf-Dieter MOLL, Getzersdorf (AT); Gerd SCHATZMAYR, Getzersdorf (AT); Sven BENSON, Stuttgart (DE); Philipp SCHELLENBERGER, Stuttgart (DE); Lenz LORENZ, Stuttgart (DE)

(21) Appl. No.: 18/685,478

(22) PCT Filed: Aug. 26, 2022
(86) PCT No.: PCT/EP2022/073780

§ 371 Date: Feb. 21, 2024

(30) Foreign Application Priority Data
Aug. 27, 2021 (EP) 21193553.1

(51) Int. Cl. C12N 9/48; A61K 38/48

(52) U.S. Cl. CPC C12N 9/485; A61K 38/4813; C12Y 304/13009

(57) ABSTRACT

The present invention relates to novel uses of the polypeptides and novel variants of said polypeptides capable of detoxifying Ochratoxin A (OTA) and methods (e.g., for detoxifying mycotoxins) based thereon. The present invention further relates to compositions, kits, transgenic plants, transgenic seeds, transgenic pollen grains, foodstuff, intermediate foodstuff; fodder, intermediate fodder; feed, intermediate feed; additive (e.g., foodstuff-, fodder- or feed additive), intermediate additive (e.g., foodstuff-, fodder- or feed intermediate additive); detoxifying agent, intermediate detoxifying agent; nutritional supplement, intermediate nutritional supplement, prebiotic, intermediate prebiotic and/or mixture/s thereof comprising one or more of the polypeptides and/or variants capable of detoxifying Ochratoxin A (OTA).

This application contains a Sequence Listing in a computer readable form, which is incorporated herein by reference.

### TECHNICAL FIELD

The present invention relates to polypeptides and variants thereof capable of detoxifying a mycotoxin (e.g., Ochratoxin A (OTA)) and methods (e.g., for detoxifying a mycotoxin, e.g., OTA) based thereon. The present invention further relates to compositions, kits, transgenic plants, transgenic seeds, transgenic pollen grains, transgenic host cells, transgenic spores, foodstuff, intermediate foodstuff; fodder, intermediate fodder; feed, intermediate feed; additive (e.g., foodstuff-, fodder- or feed additive), intermediate additive (e.g., foodstuff-, fodder- or feed intermediate additive); detoxifying agent, intermediate detoxifying agent; nutritional supplement, intermediate nutritional supplement, prebiotic, intermediate prebiotic and/or mixture/s thereof comprising one or more polypeptides and/or variants of the present invention capable of detoxifying a mycotoxin (e.g., Ochratoxin A (OTA)).

### BACKGROUND OF THE INVENTION

Mycotoxins are secondary low molecular-weight metabolites produced by fungal species mainly belonging to Aspergillus and Penicillium genera. The mycotoxin ochratoxin A (OTA; also termed e.g. N-{[(3R)-5-chloro-8-hydroxy-3-methyl-1-oxo-3,4-dihydro-1H-2-benzopyran-7-yl]carbonyl}-L-phenylalanine, (−)-N-((5-chloro-8-hydroxy-3-methyl-1-oxo-7-isochromanyl)carbonyl)-3-phenylalanine, (2S)-2-{[(3R)-5-chloro-8-hydroxy-3-methyl-1-oxo-3,4-dihydro-1H-2-benzopyran-7-carbonyl]amino}-3-phenylpropanoic acid, (R)—N-((5-chloro-3,4-dihydro-8-hydroxy-3-methyl-1-oxo-1H-2-benzopyran-7-yl)carbonyl)phenylalanine, N-(((3R)-5-chloro-8-hydroxy-3-methyl-1-oxo-7-isochromanyl)carbonyl)-3-phenyl-L-alanine, N-[(3R)-5-chloro-8-hydroxy-3-methyl-1-oxo-3,4-dihydro-1H-2-benzopyran-7-carbonyl]-L-phenylalanine, N-{[(3R)-5-chloro-8-hydroxy-3-methyl-1-oxo-3,4-dihydro-1H-isochromen-7-yl]carbonyl}-L-phenylalanine), CAS No. 303-47-9, is abundantly produced by fungi and the most toxic member of the ochratoxins group comprising inter alia ochratoxin B, ochratoxin C etc. In particular, OTA imposes serious concerns for food and feed safety due to severe adverse effects on humans and animals, including nephrotoxicity, immunotoxicity and carcinogenicity (Carballo et al., 2019; Malier et al., 2016). In this respect, OTA has been classified by the International Agency of Research on Cancer (IARC) in Group 2B as possible human carcinogen (IARC, 2012).

The environmental conditions of pre- and post-harvest stage of the edible crop, plant and related products influence the propagation of OTA-producing fungi, which lead to contamination with OTA and possibly other mycotoxins in food and feed materials. Notably, the presence of OTA has also been reported in cereal-based food and feed as well as in egg, meat, milk and products derived therefrom as a consequence of carry-over phenomena when animals were fed with contaminated feed. Further OTA presence was reported in a wide range of food items such as coffee, wine, beer, grapes, pulse, cocoa, etc. Due to the risk associated with OTA to the health and wellbeing of humans and animals, the European Commission has provided guidelines to control its presence in food (EC 1881/2006) and feed (EC 2016/1319). Therein, a tolerable weekly intake of 120 ng of OTA per kg of body weight was reported. With particular regards to animal feed, the guidance value for cereals/cereal products is 0.25 mg of OTA per kg of feed, and 0.05 mg/kg, 0.1 mg/kg and 0.01 mg/kg for compound feed for pigs, poultry and cats/dogs, respectively.

In general, a reduction of the mycotoxin content, in particular the OTA content, in food and feed materials is an unmet need of the food and feed industry striving to ensure a safe supply chain. Reports are available on the microbial detoxification of OTA using whole cells of Phenylobacterium immobile or Trichosporon mycotoxinivorans for OTA hydrolysis (Wegst and Lingens 1983; US 2009/0098244 A1). However, the applicability of microbes for OTA hydrolysis in food and feed applications is challenging due to regulatory and safety requirements. Additionally, the use of whole cell microbes is associated with complex and expensive requirements for cultivating and formulating the microbes in a manner ensuring sufficient activity of the enzymes comprised by said microbes.

As an alternative to microbial detoxification of OTA, it would be desirable to develop an enzyme-based strategy for biochemical OTA degradation. In this regard, an enzyme termed ochratoxinase from Aspergillus niger was previously reported to possess OTA hydrolyzing activity (e.g., Dobritzsch et al., 2014, Biochem J. 15; 462(3):441-52). However, an application of this enzyme under real-life conditions as required in the food and feed production line has not been shown and is considered unlikely. Dellafiora et al. (Toxins 2020, 12, 258) reported a porcine carboxypeptidase B as OTA hydrolyzing enzyme. EP 2 613 647 B1 discloses an amidase capable of degrading OTA. Notwithstanding, the OTA hydrolyzing enzymes reported so far cannot be expected to effectively perform in the application conditions due to insufficient activity, stability and/or recombinant reducibility.

Accordingly, there is a need to provide further improved OTA-hydrolyzing enzymes and variants thereof with improved functionality (e.g., enzymatic kinetics and/or efficiency) and/or stability (e.g., thermostability).

### SUMMARY OF THE INVENTION

The present invention relates to a method (e.g., in vitro, ex vivo, in vivo and/or manufacturing method, e.g., an industrial application) for detoxifying (e.g., modifying and/or hydrolyzing) a mycotoxin (e.g., an ochratoxin) having Formula I (e.g., Ochratoxin A, Ochratoxin B and/or Ochratoxin C):

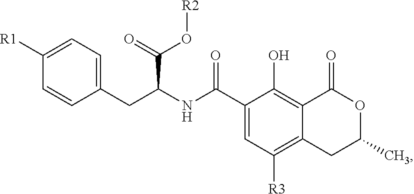

wherein R1 is selected from the group consisting of H and OH, R2 is selected from the group consisting of H and CH₂—CH₃ and R3 is selected from the group consisting of H and Cl; said method comprising: (a) providing: (i) one or more polypeptides having at least 70% sequence identity (e.g. at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) to the amino acid sequence selected from the group consisting of: SEQ ID NO: 1, SEQ ID NO: 2, SEQ ID NO: 22, SEQ ID NO: 318, SEQ ID NO: 336, SEQ ID NO: 348, SEQ ID NOs: 16-359, 368-370; preferably said one or more polypeptide are capable of detoxifying (e.g., modifying or hydrolyzing) at least one mycotoxin (e.g. ochratoxin) having said Formula I; further preferably said polypeptide/s having peptidase activity having EC 3.4.13.X, wherein X is selected from the group consisting of: 9, 4, 5, 7, 12, 17, 18, 19, 20, 21, 22, 23; most preferably said polypeptide/s having peptidase activity having EC 3.4.13.9; further most preferably said one or more polypeptide comprising one or more of the following amino acid sequences (e.g., conservative motifs): (i′)-(xxi′) as defined herein (e.g., SEQ ID NOs: 3-15, 360-367); (ii) one or more variants of a parent polypeptide, wherein said variant/s comprising an alteration (e.g., a substitution, deletion and/or insertion) at one or more positions, wherein said variant/s having at least 70% (e.g. at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%), but less than 100% sequence identity to the amino acid sequence selected from the group consisting of: SEQ ID NOs: 1, 2, 16-21, 22, 23-34, 317-318, 326-335, 336-337, 348-349, 368-370, preferably said variant/s are capable of detoxifying (e.g., modifying or hydrolyzing) at least one mycotoxin (e.g. ochratoxin) having said Formula I (e.g., Ochratoxin A, Ochratoxin B and/or Ochratoxin C); further preferably said variant/s having peptidase activity having EC 3.4.13.X, wherein X is selected from the group consisting of: 9, 4, 5, 7, 12, 17, 18, 19, 20, 21, 22, 23; most preferably said variant/s having peptidase activity having EC 3.4.13.9; further most preferably said variant/s comprising one or more of the following amino acid sequences (e.g., conservative motifs): (i′)-(xiii′) according to (i); further most preferably said parent polypeptide is selected from the group consisting of: SEQ ID NOs: 1, 2, 16-21, 22, 23-34, 317-318, 326-335, 336-337, 348-349, 368-370; (iii) one or more polynucleotides, nucleic acid constructs and/or expression vectors encoding and/or capable of expressing one or more polypeptides and/or one or more variants according to (i)-(ii); (iv) one or more recombinant host cells, spores, transgenic plants, transgenic seeds and/or transgenic pollen grains comprising (i), (ii) and/or (iii); and/or (v) foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent, intermediate detoxifying agent; nutritional supplement, intermediate nutritional supplement; prebiotic, intermediate prebiotic and/or mixture/s thereof comprising (i), (ii), (iii) and/or (iv); (b) applying (a) to said mycotoxin.

The present invention further relates to a foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent, intermediate detoxifying agent; nutritional supplement, intermediate nutritional supplement; prebiotic, intermediate prebiotic and/or mixture/s thereof, comprising one or more of the following: (i) one or more polypeptides having at least 70% sequence identity (e.g. at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) to the amino acid sequence selected from the group consisting of: SEQ ID NO: 1, SEQ ID NO: 2, SEQ ID NO: 22, SEQ ID NO: 318, SEQ ID NO: 336, SEQ ID NO: 348, SEQ ID NOs: 16-359, 368-370; preferably said polypeptide/s are capable of detoxifying (e.g., modifying or hydrolyzing) at least one mycotoxin (e.g. ochratoxin) having said Formula I as disclosed herein (e.g., Ochratoxin A, Ochratoxin B and/or Ochratoxin C); further preferably said polypeptide/s having peptidase activity having EC 3.4.13.X, wherein X is selected from the group consisting of: 9, 4, 5, 7, 12, 17, 18, 19, 20, 21, 22, 23; most preferably said polypeptide/s having peptidase activity having EC 3.4.13.9; further most preferably said polypeptide/s comprising one or more of the following amino acid sequences (e.g., conservative motifs): (i′)-(xxi′) as defined herein (e.g., SEQ ID NOs: 3-15, 360-367); (ii) one or more variants of a parent polypeptide, wherein said variant/s comprising an alteration (e.g., a substitution, deletion and/or insertion) at one or more positions, wherein said variant/s having at least 70% (e.g. at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%), but less than 100% sequence identity to the amino acid sequence selected from the group consisting of: SEQ ID NOs: 1, 2, 16-21, 22, 23-34, 317-318, 326-335, 336-337, 348-349, 368-370, preferably said variant/s are capable of detoxifying (e.g., modifying or hydrolyzing) at least one mycotoxin (e.g. ochratoxin) having said Formula I (e.g., Ochratoxin A, Ochratoxin B and/or Ochratoxin C); further preferably said variant/s having peptidase activity having EC 3.4.13.X, wherein X is selected from the group consisting of: 9, 4, 5, 7, 12, 17, 18, 19, 20, 21, 22, 23; most preferably said variant/s having peptidase activity having EC 3.4.13.9; further most preferably said variant/s comprising one or more of the following amino acid sequences (e.g., conservative motifs): (i′)-(xxi′) according to (i); further most preferably said parent polypeptide is selected from the group consisting of: (iii) one or more polynucleotides, nucleic acid constructs and/or expression vectors encoding and/or capable of expressing one or more polypeptides and/or one or more variants according to (i)-(ii); and/or (iv) one or more recombinant host cells, spores, transgenic plants, transgenic seeds and/or transgenic pollen grains comprising (i), (ii) and/or (iii).

The present invention further relates to a variant of a parent polypeptide (e.g., a parent peptidase having EC 3.4.13.X, wherein X is selected from the group consisting of: 9, 4, 5, 7, 12, 17, 18, 19, 20, 21, 22, 23; e.g., said parent peptidase having EC 3.4.13.9 and/or selected from the group consisting of: SEQ ID NOs: 1-2, SEQ ID NO: 22, SEQ ID NO: 318, SEQ ID NO: 336, SEQ ID NO: 348) capable of detoxifying (e.g., modifying or hydrolyzing) at least one mycotoxin (e.g. ochratoxin) having said Formula I as defined herein (e.g., Ochratoxin A, Ochratoxin B and/or Ochratoxin C), wherein said variant comprising an alteration (e.g., a substitution, deletion and/or insertion) at one or more positions, wherein said variant/s having at least 70% (e.g. at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%), but less than 100% sequence identity to the amino acid sequence selected from the group consisting of: SEQ ID NOs: 1, 2, 16-21, 22, 23-34, 317-318, 326-335, 336-337, 348-349, 368-370; preferably said variant having peptidase activity having EC 3.4.13.X, wherein X is selected from the group consisting of: 9, 4, 5, 7, 12, 17, 18, 19, 20, 21, 22, 23; further preferably said variant having peptidase activity having EC 3.4.13.9; most preferably, said variant comprising one or more of the following amino acid sequences (e.g., conservative motifs): (i′)-(xxi′) as defined herein (e.g., SEQ ID NOs: 3-15, 360-367).

The present application satisfies this need by the provision of polypeptides described herein below, characterized in the claims and illustrated by the appended Examples and FIGURES.

### Overview of the Sequence Listing

As described herein references can be made to UniProtKB Accession Numbers (http://www.uniprot.org/, e.g., as available in UniProt release 202101 published Feb. 10, 2021).

As described herein references can be made to GenBank Accession Numbers (https://www.ncbi.nlm.nih.gov/genbank/, e.g., as available in Release 242.0 published Feb. 15, 2021.

SEQ ID NO: 1 is the amino acid sequence of a mature species of an exemplary OTA-hydrolyzing polypeptide 1 of the present invention belonging to the amidohydrolase protein family. SEQ ID NO: 1 can be used as an exemplary parent polypeptide of the present invention.

SEQ ID NO: 2 is the amino acid sequence of an exemplary OTA-hydrolyzing polypeptide 2 of the present invention belonging to the amidohydrolase protein family. SEQ ID NO: 2 can be used as an exemplary parent polypeptide of the present invention.

SEQ ID NOs: 3-15 are exemplary amino acid motifs (e.g., conservative motifs) of the present invention.

SEQ ID NO: 16 is the amino acid sequence of an exemplary OTA-hydrolyzing polypeptide of the present invention.

SEQ ID NO: 17 is the amino acid sequence of an exemplary OTA-hydrolyzing polypeptide 3 of the present invention belonging to the amidohydrolase protein family.

SEQ ID NO: 18 is the amino acid sequence of an exemplary OTA-hydrolyzing polypeptide 4 of the present invention belonging to the amidohydrolase protein family.

SEQ ID NO: 19 is the amino acid sequence of an exemplary OTA-hydrolyzing polypeptide 5 of the present invention belonging to the amidohydrolase protein family.

SEQ ID NO: 20 is the amino acid sequence of an exemplary OTA-hydrolyzing polypeptide 6 of the present invention belonging to the amidohydrolase protein family.

SEQ ID NO: 21 is the amino acid sequence of an exemplary OTA-hydrolyzing polypeptide 7.

SEQ ID NO: 22 is the amino acid sequence of an exemplary OTA-hydrolyzing polypeptide 8 of the present invention belonging to the amidohydrolase protein family. SEQ ID NO: 22 can be used as an exemplary parent polypeptide of the present invention.

SEQ ID NOs: 23-34 are the amino acid sequences of further exemplary OTA-hydrolyzing polypeptides of the present invention belonging to the amidohydrolase protein family.

SEQ ID NOs: 35-47 are the amino acid sequences of further exemplary OTA-hydrolyzing polypeptides of the present invention belonging to the amidohydrolase protein family.

SEQ ID NOs: 48-316 are the amino acid sequences of further exemplary OTA-hydrolyzing polypeptides of the present invention belonging to the amidohydrolase protein family.

SEQ ID NOs: 317 and 318 are the amino acid sequences of another mature species and the native, non-matured species, respectively of an exemplary OTA-hydrolyzing polypeptide having the amino acid sequence of SEQ ID NO: 1 of the present invention belonging to the amidohydrolase protein family.

SEQ ID NOs: 319-325 are the amino acid sequences of further exemplary variants of the OTA-hydrolyzing polypeptide having the amino acid sequence of SEQ ID NO: 1 and further comprising alternative N-terminal leader peptides.

SEQ ID NOs: 326-335 are the amino acid sequences of mature species of either of the exemplary OTA-hydrolyzing polypeptides of SEQ ID NOs: 17-21.

SEQ ID NO: 336-337 are the amino acid sequences of another exemplary OTA-hydrolyzing polypeptides of the present invention belonging to the amidohydrolase protein family. SEQ ID NO: 336 can be used as an exemplary parent polypeptide of the present invention.

SEQ ID NOs: 338-347 are the amino acid sequences of exemplary variants of the OTA-hydrolyzing polypeptide having the amino acid sequence of SEQ ID NO: 336.

SEQ ID NOs: 348-349 are the amino acid sequence of another exemplary OTA-hydrolyzing polypeptides of the present invention belonging to the amidohydrolase protein family. SEQ ID NO: 348 can be used as an exemplary parent polypeptide of the present invention.

SEQ ID NOs: 350-359 are the amino acid sequences of exemplary variants of the OTA-hydrolyzing polypeptide having the amino acid sequence of SEQ ID NO: 348.

SEQ ID NOs: 360-367 are further exemplary amino acid motifs (e.g., conservative motifs) of the present invention.

SEQ ID NOs: 368-370 are the amino acid sequences of further exemplary OTA-hydrolyzing polypeptides of the present invention belonging to the amidohydrolase protein family.

### BRIEF DESCRIPTION OF THE DRAWINGS

FIG. 1: Exemplary scheme of Ochratoxin A hydrolysis into phenylalanine and Ochratoxin a (Dellafiora et al., Toxins 2020, 12, 258).

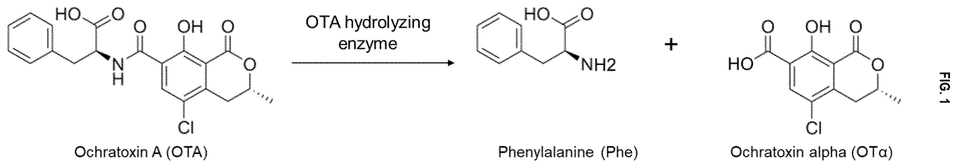

### DETAILED DESCRIPTION OF THE INVENTION

### Definitions

As referred herein “EC numbers” (Enzyme Commission numbers) may be used to refer to enzymatic activity according to the Enzyme nomenclature database, Release of Feb. 26, 2020 (e.g., available at https://enzyme.expasy.org/). The EC number refers to Enzyme Nomenclature 1992 from NC-IUBMB, Academic Press, San Diego, Calif., including supplements 1-5 published in Eur. J. Biochem. 1994, 223, 1-5; Eur. J. Biochem. 1995, 232, 1-6; Eur. J. Biochem. 1996, 237, 1-5; Eur. J. Biochem. 1997, 250, 1-6; and Eur. J. Biochem. 1999, 264, 610-650; respectively.

The term “polypeptide” is equally used herein with the term “protein”. Proteins (including fragments thereof, preferably biologically active fragments, and peptides, usually having less than 30 amino acids) comprise one or more amino acids coupled to each other via a covalent peptide bond (resulting in a chain of amino acids). The term “polypeptide(s)” as used herein describes a group of molecules, which, for example, consist of more than 30 amino acids. Polypeptides may further form multimers such as dimers, trimers and higher oligomers, i.e. consisting of more than one polypeptide molecule. Polypeptide molecules forming such dimers, trimers etc. may be identical or non-identical. The corresponding higher order structures of such multimers are, consequently, termed homo- or heterodimers, homo- or heterotrimers etc. An example for a heteromultimer is an antibody molecule, which, in its naturally occurring form, consists of two identical light polypeptide chains and two identical heavy polypeptide chains. The terms “polypeptide” and “protein” also refer to naturally modified polypeptides/proteins wherein the modification is affected e.g. by post-translational modifications like glycosylation, acetylation, phosphorylation and the like. Such modifications are well known in the art.

Amino acid motif: The term “amino acid motif” or “the motif” as used herein may refer to a specifically defined amino acid stretch of a polypeptide. Thus, an amino acid motif of the prevent invention (e.g., as shown in SEQ ID NOs: 3-15) may relate to a short sequence of amino acids within a polypeptide (e.g., within SEQ ID NO: 1 or SEQ ID NO: 2).

Sequence identity: The relatedness between two amino acid sequences or between two nucleotide sequences is described by the parameter “sequence identity”. For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used may be gap open penalty of 10, gap extension penalty of 0.5, and the EBLOSUM62 (EMBOSS version of BLOSUM62) substitution matrix. The output of Needle labeled “longest identity” (obtained using the no-brief option) or labeled “identity” is used as the percent identity and is calculated as follows:

(Identical Residues×100)/(Length of Alignment−Total Number of Gaps in Alignment).

Alternatively, the parameters used may be gap open penalty of 10, gap extension penalty of 0.5, and the EDNAFULL (EMBOSS version of NCBI NUC4.4) substitution matrix. The output of Needle labeled “longest identity” (obtained using the no-brief option) is used as the percent identity and is calculated as follows:

(Identical Deoxyribonucleotides×100)/(Length of Alignment−Total Number of Gaps in Alignment).

Expression: The term “expression” includes any step involved in the production of a variant (polypeptide) including, but not limited to, transcription, post-transcriptional modification, translation, post-translational modification, and secretion.

Expression vector: The term “expression vector” may refer to a linear or circular DNA molecule that comprises a polynucleotide encoding a variant (polypeptide) and is operably linked to control sequences that provide for its expression, in particular for its transcription.

Fragment: The term “fragment” may refer to a polypeptide having one or more (e.g. several) amino acids absent from the amino and/or carboxyl terminus of a mature polypeptide; wherein the fragment has an activity as described elsewhere herein.

Host cell: The term “host cell” may refer to any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication, e.g., recombinant or transgenic host cell.

Nucleic acid construct: The term “nucleic acid construct” may refer to a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” may refer to a configuration in which a control sequence is placed at an appropriate position relative to the coding sequence of a polynucleotide such that the control sequence directs expression of the coding sequence.

Control sequences: The term “control sequences” as used herein may refer to nucleic acid sequences necessary for expression of a polynucleotide encoding a variant (polynucleotide) of the present invention. Each control sequence may be native (i.e., from the same gene) or foreign (i.e., from a different gene) to the polynucleotide encoding the variant or native or foreign to each other. Such control sequences include, but are not limited to, a leader, polyadenylation sequence, pro-peptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide of the present invention.

As used herein, the term “corresponding to” may refer to a way of determining the specific amino acid of a sequence wherein reference is made to a specific amino acid sequence (e.g., US2020071638). E.g. for the purposes of the present invention, when references are made to specific amino acid positions, the skilled person would be able to align another amino acid sequence to said amino acid sequence that reference has been made to, in order to determine which specific amino acid may be of interest in said another amino acid sequence. Alignment of another amino acid sequence with e.g. the sequence as set forth in SEQ ID NOs: 1 or 2 or 22 or 318 or any other sequence listed herein (e.g., SEQ ID NO: 336, SEQ ID NO: 348), has been described elsewhere herein. Alternative alignment methods may be used, and are well-known for the skilled person.

The term “position” when used in accordance with the present invention may refer to a position of an amino acid within an amino acid sequence depicted herein. The term “corresponding” in this context may include that a position is not only determined by the number of the preceding nucleotides/amino acids.

As used herein, “silent” mutations mean base substitutions within a nucleic acid sequence which do not change the amino acid sequence encoded by the nucleic acid sequence. “Conservative or equivalent” substitutions (or mutations) mean substitutions as listed as “Exemplary Substitutions” in Table 1 below. “Highly conservative” substitutions as used herein mean substitutions as shown under the heading “Preferred Substitutions” in Table 1 below.

TABLE 1
Amino acid substitutions

| Original | Exemplary Substitutions | Preferred Substitutions |
|---|---|---|
| Ala (A) | val; leu; ile | val |
| Arg (R) | lys; gln; asn | lys |
| Asn (N) | gln; his; asp, lys; arg | gln |
| Asp (D) | glu; asn | glu |
| Cys (C) | ser; ala | ser |
| Gln (Q) | asn; glu | asn |
| Glu (E) | asp; gln | asp |
| Gly (G) | ala | ala |
| His (H) | asn; gln; lys; arg | arg |
| Ile (I) | leu; val; met; ala; phe; | leu |
| Leu (L) | norleucine; ile; val; met; ala; | ile |
| Lys (K) | arg; gln; asn | arg |
| Met (M) | leu; phe; ile | leu |
| Phe (F) | leu; val; ile; ala; tyr | tyr |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; ala; | leu |

Variant: The term “variant” may refer to a polypeptide having specific activity as described herein comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding an amino acid adjacent to and immediately following the amino acid occupying a position.

In describing the variants of the present invention, the nomenclature described below is adapted for ease of reference. The accepted IUPAC single letter or three letter amino acid abbreviation is employed.

Substitutions. For an amino acid substitution, the following nomenclature is used: Original amino acid, position, substituted amino acid. Accordingly, the substitution of Asn (N) at position 167 with Thr (T) is designated as “N167T” or “Asn167Thr”. Multiple mutations can be separated by addition marks (“+”) or (“,”) or (“ ”) e.g., “N167T+F168Y+S174C+F218Y;” or “N167T, F168Y, S174C, F218Y;” or “N167T/F168Y/S174C/F218Y;”, representing multiple substitutions at given positions. In the Examples of the present application, multiple mutations can be separated by comma, e.g., N167T, F168Y, S174C, F218Y. Furthermore, “X” or “Xaa” as used herein may mean any amino acid (e.g., as depicted in Table 1 above). Accordingly, “X167T” as used herein may mean substitution of any amino acid in position 167 with T (Thr). In case where the original amino acid residue may be any amino acid residue, a short hand notation may also be used indicating only the position and substituted amino acid. Accordingly, “X” or “Xaa” may be omitted in designating substitutions, e.g., “167T” designation may be used meaning a substitution of any amino acid in position 167 with T (Thr). Furthermore, “X167G,A,S,C,U,I,L,V,T” as used herein may mean substitution of any amino acid in position 167 with any one of G, A, S, C, U, I, L, V or T. In case where the substituting amino acid residue may be any amino acid residue, a short hand notation may also be used indicating only the original amino acid and its position, e.g., “N167”.

As used herein the term “transgenic” may refer to an organism whose genome has been altered by the incorporation of foreign genetic material or additional copies of native genetic material, e.g. by transformation or recombination (e.g., U.S. Pat. No. 7,410,800B2). The transgenic organism may be a plant, mammal, fungus, bacterium or virus. As used herein “transgenic plant, seed or pollen grain” may refer to a plant, seed or pollen grain or progeny plant, seed or pollen grain of any subsequent generation derived therefrom, wherein the DNA of the plant, seed or pollen grain or progeny thereof contains an introduced exogenous DNA not originally present in a non-transgenic plant, seed or pollen grain of the same strain. The transgenic plant, seed or pollen grain may additionally contain sequences which are native to the plant being transformed, but wherein the exogenous DNA has been altered in order to alter the level or pattern of expression of the coding sequence.

The term “foodstuff” may refer to a substance having a food value.

The term “fodder” may refer to a substance fed to domestic animals.

The term “feed” may refer to a substance used as food for livestock.

The term “additive” may refer to a compound or substance added to another product or substance, e.g., in a small amount, to affect a desired property and/or characteristics.

The term “prebiotic” may refer to a compound or substance capable of inducing the growth and/or activity of beneficial microorganisms.

The term “detoxifying agent” may refer to a compound or substance capable of reducing- and/or inhibiting toxicity (e.g., in a suitable form (e.g., solubilized or granulated or pelleted) and/or in a suitable solution (e.g., in a buffer solution, e.g., in sodium phosphate buffer solution) and/or under suitable conditions, e.g., as disclosed in Example 3 herein (e.g., under pH 7.5 and/or at 37° C.).

The term “nutritional supplement” may refer to a compound or substance capable to support the nutritional content of the diet, e.g., vitamins and minerals.

The term “phycophytic substance” may refer to a substance derived from a seaweed or algae species.

Amino acid motif: The term “amino acid motif” or “the motif” as used herein may refer to a specifically defined amino acid stretch of a polypeptide. Thus, an amino acid motif of the prevent invention may relate to a short sequence of amino acids within a given polypeptide.

The term “intermediate” may refer to a compound or substance produced during the process (e.g., during an intermediate stage of the process) of obtaining an end-product of the present invention, e.g., foodstuff, fodder, fodder; feed, additive (e.g., foodstuff-, fodder- or feed additive), detoxifying agent, nutritional supplement or prebiotic of the present invention.

The term “ochratoxin” may refer to a compound having Formula I (e.g., Ochratoxin A, Ochratoxin B and/or Ochratoxin C):

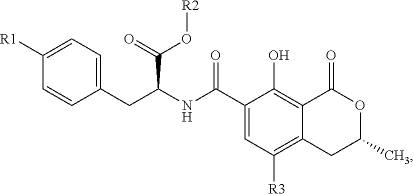

wherein R1 is selected from the group consisting of H and OH, R2 is selected from the group consisting of H and CH₂—CH₃ and R3 is selected from the group consisting of H and Cl.

It must be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

In each instance herein any of the terms “comprising”, “consisting essentially of” and “consisting of” may be replaced with either of the other two terms.

The objective of the present invention has been achieved by providing means and methods as described herein, e.g., by providing further improved polypeptides and novel variants thereof capable of detoxifying mycotoxin/s (e.g. ochratoxin/s, e.g., Ochratoxin A (OTA)), in particular polypeptides/variants, e.g., with improved thermostability and/or improved OTA degradation ability, e.g., before pelleting and/or after pelleting (e.g., as disclosed in Table 6-18 herein).

As shown in examples section below the polypeptides and variants of the present invention demonstrate applicability in industrial application of OTA detoxification in food/feed.

In some embodiments/aspects, the present invention relates to polypeptide capable of detoxifying (e.g., modifying or hydrolyzing) at least one mycotoxin (e.g. ochratoxin) having Formula I (e.g., Ochratoxin A, Ochratoxin B and/or Ochratoxin C):

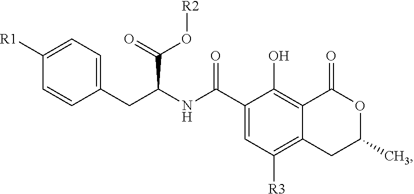

wherein R1 is selected from the group consisting of H and OH, R2 is selected from the group consisting of H and CH2-CH3 and R3 is selected from the group consisting of H and Cl.

In some embodiments/aspects, said polypeptide having a peptidase activity EC 3.4.13.X, wherein X is selected from the group consisting of: 9, 4, 5, 7, 12, 17, 18, 19, 20, 21, 22, 23; wherein said polypeptide is one or more of the following: a) a polypeptide having at least 58% sequence identity (e.g. at least 59%, 60%, 61%, 62%, 63%, 64%, 65%, 66%, 67%, 68%, 69%, 70%, 71%, 72%, 73%, 74%, 80%, 81%, 82%, 83%, 84%, 85%, 86%, 87%, 88%, 89%, 90%, 91%, 92%, 93%, 94%, 95%, 96%, 97%, 98%, 99% or 100%) to the amino acid sequence selected from the group consisting of: SEQ ID NOs: 1, SEQ ID NO: 2, SEQ ID NO: 22, SEQ ID NO: 318, SEQ ID NO: 336, SEQ ID NO: 348, SEQ ID NOs: 16-359, 368-370; b) a variant of the polypeptide of (a), wherein said variant comprising a substitution, deletion, and/or insertion at one or more positions (e.g., said variant having one or more conservative or equivalent substitutions; c) a fragment of the polypeptide of (a) or (b), wherein said fragment is capable of detoxifying the mycotoxin having said Formula I.

In some embodiments/aspects, the present invention relates to a method (e.g., in vitro, ex vivo, in vivo method and/or manufacturing method) for detoxifying (e.g., modifying or hydrolyzing) a mycotoxin (e.g., an ochratoxin) having said Formula I (e.g., Ochratoxin A, Ochratoxin B and/or Ochratoxin C); said method comprising: (a) providing: (i) one or more polypeptides having at least 70% sequence identity (e.g. at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) to the amino acid sequence selected from the group consisting of: SEQ ID NOs: 1, SEQ ID NO: 2, SEQ ID NO: 22, SEQ ID NO: 318, SEQ ID NO: 336, SEQ ID NO: 348, SEQ ID NOs: 16-359, 368-370; wherein said one or more polypeptide are capable of detoxifying (e.g., modifying or hydrolyzing) at least one mycotoxin (e.g. ochratoxin) having said Formula I; preferably said polypeptide/s having peptidase activity having EC 3.4.13.X, wherein X is selected from the group consisting of: 9, 4, 5, 7, 12, 17, 18, 19, 20, 21, 22, 23; further preferably said polypeptide/s having a peptidase activity having EC 3.4.13.9; most preferably said one or more polypeptide comprising one or more of the following amino acid sequences (e.g., conservative motifs): PGFIDAHVH (SEQ ID NO: 3), preferably said SEQ ID NO: 3 is comprised at amino acid positions corresponding to positions 87-95 of SEQ ID NO: 1 (e.g., using the numbering of SEQ ID NO: 1); PAEQA (SEQ ID NO: 4), preferably said SEQ ID NO: 4 is comprised at amino acid positions corresponding to positions 116-120 of SEQ ID NO: 1 (e.g., using the numbering of SEQ ID NO: 1), GVCNG (SEQ ID NO: 5), preferably said SEQ ID NO: 5 is comprised at amino acid positions corresponding to positions 197-201 of SEQ ID NO: 1 (e.g., using the numbering of SEQ ID NO: 1); CRAAVR (SEQ ID NO: 6), preferably said SEQ ID NO: 6 is comprised at amino acid positions corresponding to positions 205-210 of SEQ ID NO: 1 (e.g., using the numbering of SEQ ID NO: 1); IKFMPSGGVLSL (SEQ ID NO: 7), preferably said SEQ ID NO: 7 is comprised at amino acid positions corresponding to positions 220-231 of SEQ ID NO: 1 (e.g., using the numbering of SEQ ID NO: 1); RKVAAH (SEQ ID NO: 8), preferably said SEQ ID NO: 8 is comprised at amino acid positions corresponding to positions 257-262 of SEQ ID NO: 1 (e.g., using the numbering of SEQ ID NO: 1); AGVDSIEHG (SEQ ID NO: 9), preferably said SEQ ID NO: 9 is comprised at amino acid positions corresponding to positions 275-283 of SEQ ID NO: 1 (e.g., using the numbering of SEQ ID NO: 1); VMPGLIDAH (SEQ ID NO: 10), preferably said SEQ ID NO: 10 is comprised at amino acid positions corresponding to positions 51-59 of SEQ ID NO: 2 (e.g., using the numbering of SEQ ID NO: 2); AGFTTVRDCG (SEQ ID NO: 11), preferably said SEQ ID NO: 11 is comprised at amino acid positions corresponding to positions 96-105 of SEQ ID NO: 2 (e.g., using the numbering of SEQ ID NO: 2); QTFGH (SEQ ID NO: 12), preferably said SEQ ID NO: 12 is comprised at amino acid positions corresponding to positions 135-139 of SEQ ID NO: 2 (e.g., using the numbering of SEQ ID NO: 2); ATGGVLS (SEQ ID NO: 13), preferably said SEQ ID NO: 13 is comprised at amino acid positions corresponding to positions 185-191 of SEQ ID NO: 2 (e.g., using the numbering of SEQ ID NO: 2); YVAAHAHGD (SEQ ID NO: 14), preferably said SEQ ID NO: 14 is comprised at amino acid positions corresponding to positions 219-227 of SEQ ID NO: 2 (e.g., using the numbering of SEQ ID NO: 2); LTPTL (SEQ ID NO: 15), preferably said SEQ ID NO: 15 is comprised at amino acid positions corresponding to positions 262-266 of SEQ ID NO: 2 (e.g., using the numbering of SEQ ID NO: 2); VMPGLID (SEQ ID NO: 360), preferably said SEQ ID NO: 360 is comprised at amino acid positions corresponding to positions 52-58 of SEQ ID NO: 22 (e.g., using the numbering of SEQ ID NO: 22); KGGSL (SEQ ID NO: 361), preferably said SEQ ID NO: 361 is comprised at amino acid positions corresponding to positions 67-71 of SEQ ID NO: 22 (e.g., using the numbering of SEQ ID NO: 22); LLLAGFTTVRDCG (SEQ ID NO: 362), preferably said SEQ ID NO: 362 is comprised at amino acid positions corresponding to positions 93-105 of SEQ ID NO: 22 (e.g., using the numbering of SEQ ID NO: 22); QTFGHGE (SEQ ID NO: 363), preferably said SEQ ID NO: 363 is comprised at amino acid positions corresponding to positions 135-141 of SEQ ID NO: 22 (e.g., using the numbering of SEQ ID NO: 22); FATGGVLSQRD (SEQ ID NO: 364), preferably said SEQ ID NO: 364 is comprised at amino acid positions corresponding to positions 184-194 of SEQ ID NO: 22 (e.g., using the numbering of SEQ ID NO: 22); IVNEA (SEQ ID NO: 365), preferably said SEQ ID NO: 365 is comprised at amino acid positions corresponding to positions 209-213 of SEQ ID NO: 22 (e.g., using the numbering of SEQ ID NO: 22); IGVDEWGL (SEQ ID NO: 366), preferably said SEQ ID NO: 366 is comprised at amino acid positions corresponding to positions 279-286 of SEQ ID NO: 22 (e.g., using the numbering of SEQ ID NO: 22); GFETGL (SEQ ID NO: 367), preferably said SEQ ID NO: 367 is comprised at amino acid positions corresponding to positions 317-322 of SEQ ID NO: 22 (e.g., using the numbering of SEQ ID NO: 22; (b) applying (a) to said mycotoxin (e.g. ochratoxin) having Formula I.

In some embodiments/aspects, the present invention relates to a method (e.g., in vitro, ex vivo, in vivo and/or manufacturing method) for detoxifying (e.g., modifying or hydrolyzing) a mycotoxin (e.g., an ochratoxin) having said Formula I (e.g., Ochratoxin A, Ochratoxin B and/or Ochratoxin C); said method comprising: (a) providing: ii) one or more variants of a parent polypeptide, wherein said variant/s comprising an alteration (e.g., a substitution, deletion and/or insertion) at one or more positions corresponding to positions of the parent polypeptide, wherein said variant/s having at least 70% (e.g. at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%), but less than 100% sequence identity to a parent amino acid sequence selected from the group consisting of: SEQ ID NOs: 1, 2, 16-21, 22, 23-34, 317-318, 326-335, 336-337, 348-349, 368-370; wherein said variant/s are capable of detoxifying (e.g., modifying or hydrolyzing) at least one mycotoxin (e.g. ochratoxin) having said Formula I (e.g., Ochratoxin A, Ochratoxin B and/or Ochratoxin C); preferably said variant/s having peptidase activity having EC 3.4.13.X, wherein X is selected from the group consisting of: 9, 4, 5, 7, 12, 17, 18, 19, 20, 21, 22, 23; further preferably said variant/s having a peptidase activity having EC 3.4.13.9; most preferably said variant/s comprising one or more of the following amino acid sequences (e.g., conservative motifs): (i′)-(xiii′) as defined herein; further most preferably said parent polypeptide is selected from the group consisting of: SEQ ID NOs: 1, 2, 16-21, 22, 23-34, 317-318, 326-335, 336-337, 348-349, 368-370; (b) applying (a) to said mycotoxin (e.g. ochratoxin (having Formula I.

In some embodiments/aspects, the present invention relates to a method (e.g., in vitro, ex vivo, in vivo and/or manufacturing method) for detoxifying (e.g., modifying or hydrolyzing) a mycotoxin having said Formula I (e.g., Ochratoxin A, Ochratoxin B and/or Ochratoxin C); said method comprising: (a) providing: (iii) one or more polynucleotides, nucleic acid constructs and/or expression vectors encoding and/or capable of expressing one or more polypeptides and/or one or more variants according to (i)-(ii) as above; (b) applying (a) to said mycotoxin (e.g. ochratoxin) having Formula I.

In some embodiments/aspects, the present invention relates to a method (e.g., in vitro, ex vivo, in vivo and/or manufacturing method) for detoxifying (e.g., modifying or hydrolyzing) a mycotoxin (e.g. ochratoxin) having said Formula I (e.g., Ochratoxin A, Ochratoxin B and/or Ochratoxin C); said method comprising: (a) providing: (iv) one or more recombinant host cells, spores, transgenic plants, transgenic seeds and/or transgenic pollen grains comprising (i), (ii) and/or (iii) as above; (b) applying (a) to said mycotoxin having Formula I.

In some embodiments/aspects, the present invention relates to a method (e.g., in vitro, ex vivo, in vivo and/or manufacturing method) for detoxifying (e.g., modifying or hydrolyzing) a mycotoxin (e.g. ochratoxin) having said Formula I (e.g., Ochratoxin A, Ochratoxin B and/or Ochratoxin C); said method comprising: (a) providing: (v) foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent, intermediate detoxifying agent; nutritional supplement, intermediate nutritional supplement; prebiotic, intermediate prebiotic and/or mixture/s thereof comprising (i), (ii), (iii) and/or (iv) as above; (b) applying (a) to said mycotoxin having Formula I.

In some embodiments/aspects of the present invention, the polypeptide and/or variant having a TIM barrel structure comprising 8 α-helices and 8 parallel β-strands alternating along the polypeptide backbone, wherein preferably said TIM barrel further comprising a phosphate binding site.

In some embodiments/aspects of the present invention, the polypeptide and/or variant having a specific activity of at least 2.9 U/g at pH7.5;

In some embodiments/aspects of the present invention, the polypeptide and/or variant having a specific activity of at least 0.9 U/g at pH6.0.

In some embodiments/aspects of the present invention, the polypeptide and/or variant having a T50 value of more than 80° C. (e.g. more than 85° C.).

In some embodiments/aspects of the present invention, the polypeptide is selected from the group consisting of: SEQ ID NOs: 1, 2, 16-21, 22, 23-34, 317-318, 326-335, 336-337, 348-349, 368-370.

In some embodiments/aspects of the present invention, the variant of the present invention is selected from the group consisting of: SEQ ID NOs: 35-316, 319-325, 338-347, 350-359.

In some embodiments/aspects of the present invention, the variant comprising an alteration (e.g., a substitution, deletion and/or insertion) at one or more positions corresponding to positions 65,106,132, 268, 269, 272, 275 and/or 318 of SEQ ID NO: 2, preferably using the numbering of SEQ ID NO: 2, for example, the variant comprising one or more of the following substitutions or combinations of substitutions: I65G, I65A, E106G, E106S, P132A, I268A, Q269I, I272V, Y275V; F318V; I272V; Y275F; Y275H; and/or F318V; or equivalent amino acid substitutions thereof.

In some embodiments/aspects of the present invention, the variant comprising an alteration (e.g., a substitution, deletion and/or insertion) at one or more positions corresponding to positions 30, 36, 50, 85, 97, 98, 99, 100, 103, 104, 105, 106, 107, 108, 109, 111, 112, 113, 119, 122, 126, 130, 142, 143, 144, 170, 171, 176, 178, 179, 181, 183, 184, 187, 188, 192, 198, 223, 226, 228, 229, 230, 231, 233, 235, 239, 242, 253, 264, 284, 287, 307, 308, 310, 311, 314, 315, 317, 318, 322, 329, 330, 332, 333, 339, 353, 354, 355, 356, 357, 358 or 361 of SEQ ID NO: 318 or 1, preferably using the numbering of SEQ ID NO: 318, for example, variant comprising one or more of the following substitutions or combinations of substitutions: Y300C+L389C; I308V; A171P; T144A; V355L; I308A; G354A; S143E; M223C; A171G; H187N; I184V; H187Q; E239Q; V311I; K314Q; F109A; Y122R; F318Y; A36C+L85C; A375P; M223C; M223A; H264Q; A307G; H178Q; A307G+V355T; T144A; A171P; A171G; M223C; V228A; L229V; L229M; A307S; A307G; I308V; V311T; V355T; V311A; V355A; H264Q; M223C; M223A; A171L; A171G; H178Q; H178L; A307G+V311A; A307G+V311T; A307G+V355T; A307G+A355A; H178S; V228L; V2281; L229I; H264N; H264L; A307V; A307T; I308A; V311L; V311I; G354A; S97D; S97N; S97P; S97T; S97V; G142A; G142P; T144D; T144E; T144P; T144R; A179P; A179S; A179T; A171P+I184V; A171P+I308A; A171P+I308V; A171P+F318Y; A171P+G354A; I184V+I308A; I184V+I308V; I184V+F318Y; I184V+G354A; I308A+F318Y; I308V+F318Y; I308A+G354A; I308V+G354A; F318Y+G354A; A171P+I184V+I308A; A171P+I184V+I308V; A171P+I184V+F318Y; A171P+I184V+G354A; A171P+I308A+F318Y; A171P+I308V+F318Y; A171P+I308A+G354A; A171P+I308V+G354A; A171P+F318Y+G354A; I184V+I308A+F318Y; I184V+I308V+F318Y; I184V+I308A+G354A; I184V+I308V+G354A; I184V+F318Y+G354A; I308A+F318Y+G354A; I308V+F318Y+G354A; Q181M; Q181L; F318L; F318M; A329G; A329V; V355L; V355I; Y170E; Y170F; Y170R; A330L; A330M; A330V; L333I; L333M; L333V; G354L; G354S; G354T; G354V; G356A; G356F; G356P; G356V; G356Y; F109A; P183G; P183S; F108V; F108L; F109V; F109L; M112I; M112W; I184V; I184F; W104F; W104L; W104P; W104Y; I322L; I322M; I322Q; I322S; H187Q; H187N; H310Q; H310N; K314Q; K314E; H332Q; H332V; E287Q; H358Q; H358Y; Q99D; H30E; H30K; H30P; H30R; H50D; H50N; H50P; S100D; N103D; N103L; N103Q; T130E; H187D; H187E; H187G; H187P; K188D; K188E; K188R; G226A; G226P; G226S; S230D; S230P; S230T; D233N; D233Q; D233S; H253A; H253N; S284D; S284H; S284T; H310D; H310E; H332E; H332R; H332S; H339E; H339K; H339N; H339S; A353S; H358A; H358K; H358N; N361E; N361Q; D110K+K188D; D110K+K188E; H187N+K188R; S284D+A329S; S284D+A329T; H310D+E313A; H310E+E313A; V235P; E239Q; A357P; F108Y; N111D; M113Q; Y122R; Y126W; F109Y; Q242P; I315P; T317Y; F318Y; G98S; G98V; Y105N; Y105R; G106P; D107R; D107Y; M112F; M112K; M113D; M113R; Q119E; Q119H; Q119K; Q119M; G176S; A192L; A192M; A192V; V198T; L231F; V235A; V235D; V235F; V235L; L231E+V321T; L229I+I308A; I308A+A329V; I308A+G356F; I308A+G356P; I308A+G356Y; L229I+I308V; I308V+A329V; I308V+G356F; I308V+G356P; I308V+G356Y; G354A+G356V; G354A+G356Y; H310D+V355L; H310E+V355L; V311A+V355L; G354P+V355L; V355L+G356P; I184V+A192L; I184V+A192M; S143E; N361W; I308A+A330L; I308A+A330M; I308A+A330V; I308A+L333I; I308A+L333M; I308V+A330L; I308V+A330M; I308V+A330V; I308V+L333I; I308V+L333M; I308V+V355L; F318Y+V355L; H253E+D278R; K258Y+G391R; A36C+L85C; S1000+T3170; A179C+M223C; Y300C+L389C; A309C+A330C or equivalent amino acid substitutions thereof.

In some embodiments/aspects, the present invention relates to polynucleotide, nucleic acid construct or expression vector encoding and/or capable of expressing one or more variants and/or polypeptides of the present invention.

In some embodiments/aspects, the present invention relates to a recombinant host cell (e.g., an isolated recombinant host cell), spore, transgenic plant, transgenic seed or transgenic pollen grain comprising one or more of the following: (i) one or more variants and/or polypeptides of the present invention; (ii) one or more polynucleotides of the present invention; and/or (iii) one or more nucleic acid constructs and/or expression vectors of the present invention.

In some embodiments/aspects, the present invention relates to a composition or kit comprising one or more of the following: variants, polypeptides, nucleic acid constructs, expression vectors, recombinant host cells, spores, transgenic plants, transgenic seeds and/or transgenic pollen grains, foodstuff, intermediate foodstuff; fodder, intermediate fodder; feed, intermediate feed; additive (e.g., foodstuff-, fodder- or feed additive), intermediate additive (e.g., foodstuff-, fodder- or feed intermediate additive); detoxifying agent, intermediate detoxifying agent; nutritional supplement, intermediate nutritional supplement, prebiotic, intermediate prebiotic and/or mixture/s thereof of the present invention and/or one or more polypeptides having at least 70% sequence identity (e.g. at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) to the amino acid sequence selected from the group consisting of: SEQ ID NOs: 1, 2, 16-21, 22, 23-34, 317-318, 326-335, 336-337, 348-349, 368-370; wherein said polypeptide/s are capable of detoxifying (e.g., modifying or hydrolyzing) at least one mycotoxin (e.g. ochratoxin) having said Formula I. preferably said polypeptide/s having a peptidase activity having EC 3.4.13.X, wherein X is selected from the group consisting of: 9, 4, 5, 7, 12, 17, 18, 19, 20, 21, 22, 23; further preferably said polypeptide/s having a peptidase activity having EC 3.4.13.9; most preferably said polypeptide/s comprising one or more of the following amino acid sequences (e.g., conservative motifs): (i′)-(xxi′) as defined herein.

In some further aspects/embodiments, the composition or kit of the present invention further comprising one or more of the following: (i) one or more further polypeptides capable of detoxifying a mycotoxin (e.g. ochratoxin) of said Formula I, further preferably said further one or more polypeptides belonging to the M20 Peptidase aminoacylase 1-like protein 2-like amidohydrolase subfamily (e.g., M20 Peptidase ACY1L2 amidohydrolase subfamily, e.g., having identifier cd05672 according to the Conserved Domain Database (e.g., https://www.ncbi.nlm.nih.gov/Structure/cdd/cddsrv.cgi?uid=cd05672), and/or having aminopeptidase activity (e.g., EC 3.4.11.10), and/or comprising a carboxypeptidase activity (e.g., carboxypeptidase A and/or B activity, e.g., having EC 3.4.17.1 and/or EC 3.4.17.2 respectively) and/or thermolysin activity (e.g., having EC 3.4.24.27); (ii) one or more further polypeptides capable of detoxifying one or more mycotoxins (e.g., ZEN and/or trichothecene mycotoxin/s such as e.g. deoxynivalenol, nivalenol, neosolaniol, trichotecin, crotocin, roridin A, satratoxin H, diacetoxyscirpenol, HT-2 toxin or T-2 toxin; aflatoxins such as e.g. aflatoxin B1, B2, G1 or G2; fumonisins such as e.g. fumonisin B1, B2, B3 or B4; polypeptide mycotoxins such as e.g. beauvericin or enniatins; zearalenone; citrinin; patulin; ergot alkaloids such as e.g. ergotamine) and/or one or more plant- and/or bacteria-derived toxins (e.g. endotoxin, etc.), in particular said one or more further polypeptides capable of detoxifying one or more further mycotoxins and/or one or more plant- and/or bacteria-derived toxins, e.g., a fumonisin esterase (e.g. as disclosed in WO 2016/134387 A1) and/or a zearalenone lactonase (e.g. as disclosed in WO 2020/025580 A1) and/or an ergopeptine hydrolase (e.g. as disclosed in WO 2014/056006 A1); (iii) one or more organic absorbents (e.g., live, inactivated, lyophilized, dormant, and/or dead whole-yeast or yeast-derived product such as e.g. yeast cell wall, or yeast oligosaccharides such as e.g. mannan) and/or one or more inorganic absorbents (e.g., diatomaceous earth and/or clay mineral such as e.g. kaolins or kaolinites, smectites such as e.g. montmorillonites, illites or chlorites; in particular bentonite); (iv) one or more live, inactivated, lyophilized and/or dormant microorganisms capable of detoxifying one or more further mycotoxins (e.g., trichothecene mycotoxins such as e.g. deoxynivalenol, nivalenol, neosolaniol, trichotecin, crotocin, roridin A, satratoxin H, diacetoxyscirpenol, HT-2 toxin or T-2 toxin; aflatoxins such as e.g. aflatoxin B1, B2, G1 or G2; fumonisins such as e.g. fumonisin B1, B2, B3 or B4; polypeptide mycotoxins such as e.g. beauvericin or enniatins; zearalenone; citrinin; patulin; ergot alkaloids such as e.g. ergotamine) and/or one or more plant- or bacteria-derived toxins (e.g. endotoxin, etc.), in particular said microorganism is selected from the group consisting of: Trichosporon and Apiotrichum genera (e.g. as disclosed in WO 03/053161 A1) and the Coriobacteriaceae family (e.g. as disclosed in EP 3 501 526 A1); (v) one or more plant products (e.g., seaweed, preferably seaweed meal; and/or algae, preferably algae meal; and/or thistle, preferably thistle seeds; and/or glycyrrhiza plant preparation, preferably glycyrrhiza meal and/or glycyrrhiza extract e.g. as disclosed in WO 2018/121881 A1); (vi) one or more flavoring compounds (e.g., plant extract e.g. from oregano, thyme, wintergreen, caraway, marjoram, mint, peppermint, anise, orange, lemon, fennel, star anise, clove, cinnamon and/or garlic; and/or essential oil such as e.g. D-limonene, γ-terpinene, p-cymene, 2-carene, linalool oxide, isomenthone, camphor, linalool, terpinen-4-ol, 2-isopropyl-1-methoxy-4-methylbenzene, L-menthol, ethylamine, α-terpineol, β-caryophyllene, D-carvone, methyl salicylate, α-caryophyllene, lavandulyl acetate, caryophyllene oxide, eugenol, thymol and/or carvacrol); (vii) one or more vitamins (e.g. vitamin A, D, E, K, C, B1, B2, B3, B4, B5, B6, B7, B8, B9 and/or B12; in particular vitamin E).

In particular aspects/embodiments, the composition or kit of the present invention further comprising one or more of the following: bentonite, fumonisin esterase and/or a zearalenone lactonase, a Coriobacteriaceae microorganism (e.g., a microorganism selected from the family Coriobacteriaceae, e.g., https://lpsn.dsmz.de/family/coriobacteriaceae) capable of detoxifying one or more mycotoxins, diatomaceous earth, yeast (in particular, inactivated yeast), seaweed meal, thistle seeds, and one or more flavoring compound.

In some further embodiments, compositions (e.g., exemplary compositions 1-24 as depicted in Table 2 below) or kits (corresponding to exemplary compositions 1-24 as depicted in Table 2 below) of the present invention comprising one or more further components (e.g., in addition to at least one polypeptide according to the present invention). Such further exemplary compositions or kits are explicitly disclosed in Table 2 herein below, which describes embodiments of the present invention.

TABLE 2
Exemplary compositions or kits of the present invention comprising
one or more further components.

| | Composition Number | | | | | |
|---|---|---|---|---|---|---|
| | 1 | 2 | 3 | 4 | 5 | 6 |
| Any variant or | yes | yes | yes | yes | yes | yes |
| polypeptide according | | | | | | |
| to the present | | | | | | |
| invention (e.g., | | | | | | |
| SEQ ID NOs: 1-2, | | | | | | |
| 16-359, 368-370) | | | | | | |
| Any suitable | | | | e.g., fumonisin | e.g., fumonisin | |
| polypeptide capable | | | | esterase, e.g., | esterase, e.g., | |
| of detoxifying one or | | | | having | having | |
| more further myco-/ | | | | EC: 3.1.1.87 | EC: 3.1.1.87 | |
| plant-/bacterial-toxins | | | | enzymatic | enzymatic | |
| | | | | activity, | activity, | |
| | | | | e.g., UniProtKB- | e.g., UniProtKB- | |
| | | | | D2D3B6 and/or | D2D3B6 and/or | |
| | | | | variant/s thereof | variant/s thereof | |
| Any suitable organic | | e.g., | e.g., | e.g., yeast | | |
| absorbent | | inactivated | dried yeast | | | |
| | | yeast | | | | |
| Any suitable inorganic | e.g., | e.g., | e.g., | e.g., bentonite | e.g., bentonite | e.g., |
| absorbent | bentonite | zeolite | bentonite- | | | bentonite |
| | | | montmorillonite | | | |
| Any suitable | e.g., | | | e.g., Corio- | e.g., Corio- | |
| microorganism | Corio- | | | bacteriaceae | bacteriaceae | |
| capable of detoxyifying | bacteriaceae | | | | | |
| one or more further | | | | | | |
| mycotoxins | | | | | | |
| Any suitable plant | | | e.g., algae | e.g., thistle | | |
| product | | | powder | seed | | |
| Any suitable vitamin | | | | e.g., Vitamin E | | |
| Any suitable flavoring | | | | yes | | |
| compound | | | | | | |
| | Composition Number | | | | | |
| | 7 | 8 | 9 | 10 | 11 | 12 |
| Any variant or | yes | yes | yes | yes | yes | yes |
| polypeptide according | | | | | | |
| to the present | | | | | | |
| invention (e.g., | | | | | | |
| SEQ ID NOs: 1-2 and/or | | | | | | |
| 16-359, 368-370) | | | | | | |
| Any polypeptide capable | | | e.g., fumonisin | e.g., fumonisin | e.g., fumonisin | e.g., fumonisin |
| of detoxifying one or | | | esterase, e.g., | esterase, e.g., | esterase, e.g., | esterase, e.g., |
| more further myco-/ | | | having | having | having | having |
| plant-/bacteria-toxins | | | EC: 3.1.1.87 | EC: 3.1.1.87 | EC: 3.1.1.87 | EC: 3.1.1.87 |
| | | | enzymatic | enzymatic | enzymatic | enzymatic |
| | | | activity, e.g., | activity, e.g., | activity, e.g., | activity, e.g., |
| | | | UniProtKB- | UniProtKB- | UniProtKB- | UniProtKB- |
| | | | D2D3B6 and/or | D2D3B6 and/or | D2D3B6 and/or | D2D3B6 and/or |
| | | | variant/s thereof | variant/s thereof | variant/s thereof | variant/s thereof |
| Any suitable organic | | e.g., | | e.g., | | |
| absorbent | | inactivated | | Saccharomyces | | |
| | | yeast | | cerevisiae | | |
| Any suitable inorganic | e.g., | e.g., | e.g., bentonite | e.g., bentonite | e.g., bentonite | e.g., bentonite |
| absorbent | bentonite | bentonite | | | | |
| Any suitable | e.g., | | e.g., Corio- | e.g., | e.g., Corio- | |
| microorganism | Eubacterium | | bacteriaceae | Eubacterium sp. | bacteriaceae | |
| capable of detoxyifying | sp. | | | | | |
| one or more further | | | | | | |
| mycotoxin | | | | | | |
| Any suitable plant | e.g., algae | e.g., seaweed | | e.g., phycophytic | | |
| product | extract | meal, milk | | substances | | |
| | | thistle | | | | |
| Any suitable vitamin | | | | | | |
| Any suitable flavoring | | | | | | |
| compound | | | | | | |
| | Composition number | | | | | |
| | 13 | 14 | 15 | 16 | 17 | 18 |
| Any variant or | yes | yes | yes | yes | yes | yes |
| polypeptide according | | | | | | |
| to the present | | | | | | |
| invention (e.g., | | | | | | |
| SEQ ID NOs: 1-2 and/or | | | | | | |
| 16-359, 368-370) | | | | | | |
| Any suitable | | | | e.g., fumonisin | e.g., fumonisin | |
| polypeptide capable | | | | esterase, e.g., | esterase, e.g., | |
| of detoxifying one or | | | | having | having | |
| more further myco-/ | | | | EC: 3.1.1.87 | EC: 3.1.1.87 | |
| plant-/bacteria-toxins | | | | enzymatic | enzymatic | |
| | | | | activity, e.g., | activity, e.g., | |
| | | | | UniProtKB- | UniProtKB- | |
| | | | | D2D3B6 and/or | D2D3B6 and/or | |
| | | | | variant/s thereof, | variant/s thereof, | |
| | | | | zearalenone | zearalenone | |
| | | | | lactonase, e.g., | lactonase, e.g., | |
| | | | | UniProtKB- | UniProtKB- | |
| | | | | Q8NKB0 and/or | Q8NKB0 and/or | |
| | | | | variant/s thereof | variant/s thereof | |
| Any suitable organic | | e.g., | e.g., dried | e.g., yeast | | |
| absorbent | | inactivated | yeast | | | |
| | | yeast | | | | |
| Any suitable inorganic | e.g., | e.g., | e.g., bentonite- | e.g., | e.g., | e.g., |
| absorbent | bentonite | zeolite | montmorillonite | bentonite | bentonite | bentonite |
| Any suitable | e.g., Corio- | | | e.g., Corio- | e.g., Corio- | |
| microorganism | bacteriaceae | | | bacteriaceae | bacteriaceae | |
| capable of detoxyifying | | | | | | |
| one or more further | | | | | | |
| mycotoxins | | | | | | |
| Any suitable plant | | | e.g., algae | | | |
| product | | | powder | | | |
| Any suitable vitamin | | | | e.g., Vitamin E | | |
| Any suitable flavoring | | | | yes | | |
| compound | | | | | | |
| | Composition number | | | | | |
| | 19 | 20 | 21 | 22 | 23 | 24 |
| Any variant or | yes | yes | yes | yes | yes | yes |
| polypeptide according | | | | | | |
| to the present | | | | | | |
| invention (e.g., | | | | | | |
| SEQ ID NOs: 1-2 and/or | | | | | | |
| 16-359, 368-370) | | | | | | |
| Any polypeptide capable | | | fumonisin | fumonisin | fumonisin | fumonisin |
| of detoxifying one or | | | esterase, e.g., | esterase, e.g., | esterase, e.g., | esterase, e.g., |
| more further myco-/ | | | having | having | having | having |
| plant-/bacteria-toxins | | | EC: 3.1.1.87 | EC: 3.1.1.87 | EC: 3.1.1.87 | EC: 3.1.1.87 |
| | | | enzymatic | enzymatic | enzymatic | enzymatic |
| | | | activity, e.g., | activity, e.g., | activity, e.g., | activity, e.g., |
| | | | UniProtKB- | UniProtKB- | UniProtKB- | UniProtKB- |
| | | | D2D3B6 and/or | D2D3B6 and/or | D2D3B6 and/or | D2D3B6 and/or |
| | | | variant/s thereof, | variant/s thereof, | variant/s thereof, | variant/s thereof, |
| | | | zearalenone | zearalenone | zearalenone | zearalenone |
| | | | lactonase, e.g., | lactonase, e.g., | lactonase, e.g., | lactonase, e.g., |
| | | | UniProtKB- | UniProtKB- | UniProtKB- | UniProtKB- |
| | | | Q8NKB0 and/or | Q8NKB0 and/or | Q8NKB0 and/or | Q8NKB0 and/or |
| | | | variant/s thereof | variant/s thereof | variant/s thereof | variant/s thereof |
| Any suitable organic | | e.g., | | e.g., | | |
| absorbent | | inactivated | | Saccharomyces | | |
| | | yeast | | cerevisiae | | |
| Any suitable inorganic | e.g., | e.g., | e.g., | e.g., | e.g., | e.g., |
| absorbent | bentonite | bentonite | bentonite | bentonite | bentonite | bentonite |
| Any suitable | e.g., | | e.g., Corio- | e.g., | e.g., Corio- | |
| microorganism | Eubacterium | | bacteriaceae | Eubacterium sp. | bacteriaceae | |
| capable of detoxyifying | sp. | | | | | |
| one or more further | | | | | | |
| mycotoxins | | | | | | |
| Any suitable plant | e.g., algae | e.g., seaweed | | e.g., phycophytic | | |
| product | extract | meal, milk | | substances | | |
| | | thistle | | | | |
| Any suitable vitamin | | | | | | |
| Any suitable flavoring | | | | | | |
| compound | | | | | | |

In some embodiments/aspects, the present invention relates to a method for producing a foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); —detoxifying agent, intermediate detoxifying agent; nutritional supplement, intermediate nutritional supplement; prebiotic, intermediate prebiotic and/or mixture/s thereof, said method comprising: a) providing: (i) one or more polypeptides having at least 70% sequence identity (e.g. at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) to the amino acid sequence selected from the group consisting of: SEQ ID NO: 1, SEQ ID NO: 2, SEQ ID NO: 22, SEQ ID NO: 318, SEQ ID NO: 336, SEQ ID NO: 348, SEQ ID NO: 17-21, 23-34, 316-359, 368-370; wherein said polypeptide/s are capable of detoxifying (e.g., modifying or hydrolyzing) at least one mycotoxin (e.g. ochratoxin) having said Formula I; preferably said polypeptide/s having a peptidase activity having EC 3.4.13.X, wherein X is selected from the group consisting of: 9, 4, 5, 7, 12, 17, 18, 19, 20, 21, 22, 23; further preferably said polypeptide/s having a peptidase activity having EC 3.4.13.9; most preferably said polypeptide/s comprising one or more of the following amino acid sequences (e.g., conservative motifs): (i′)-(xxi′) as defined herein; (ii) variant/s according of the present invention; (iii) one or more polynucleotides of the present invention; (iv) one or more nucleic acid constructs and/or expression vectors of the present invention; and/or (v) one or more recombinant host cells, spores, transgenic plants, transgenic seeds and/or transgenic pollen grains of the present invention; preferably said one or more polypeptides are one or more recombinant and/or isolated polypeptides; b) applying (a) to a nutritive source or material suitable for production of foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent, intermediate detoxifying agent; nutritional supplement, intermediate nutritional supplement; prebiotic, intermediate prebiotic and/or mixture/s thereof.

In some embodiments/aspects, the present invention relates to a foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent, intermediate detoxifying agent; nutritional supplement, intermediate nutritional supplement; prebiotic, intermediate prebiotic and/or mixture/s thereof produced by the method of the present invention.

In some embodiments/aspects, the present invention relates to a variant, polypeptide, polynucleotide, nucleic acid construct, expression vector, recombinant host cell, spore, transgenic plant, transgenic seed, transgenic pollen grain, foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent, intermediate detoxifying agent; nutritional supplement, intermediate nutritional supplement; prebiotic, intermediate prebiotic or mixture/s thereof, composition or kit of the present invention for use as a medicament (e.g., for veterinary use) and/or in therapy and/or prophylaxis of a disease.

In some embodiments/aspects, the present invention relates to a variant, polypeptide, polynucleotide, nucleic acid construct, expression vector, recombinant host cell, spore, transgenic plant, transgenic seed, transgenic pollen grain, foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent, intermediate detoxifying agent; nutritional supplement, intermediate nutritional supplement; prebiotic, intermediate prebiotic or mixture/s thereof, composition or kit of the present invention for use for use in one or more of the following methods: method for treatment, amelioration, prophylaxis and/or diagnostics of mycotoxicosis, preferably an OTA mycotoxicosis; method for monitoring development of mycotoxicosis and/or assessing the efficacy of a mycotoxicosis prophylaxis and/or therapy, preferably an OTA mycotoxicosis prophylaxis and/or therapy; method for detoxifying and/or altering toxicity of a mycotoxin (e.g. ochratoxin) having Formula I; method of producing one or more of the following: foodstuff, intermediate foodstuff; fodder, intermediate fodder; feed intermediate feed; additive (e.g., foodstuff-, fodder- or feed additive), intermediate additive (e.g., foodstuff-, fodder- or feed intermediate additive); detoxifying agent, intermediate detoxifying agent; nutritional supplement, intermediate nutritional supplement; prebiotic, intermediate prebiotic; pharmaceutical, veterinary, diagnostic, detoxifying, monitoring and/or screening composition or kit; method according to any one of the preceding items; any combination of methods as above; any method as define herein, wherein said method is an in vitro, ex vivo, in vivo and/or manufacturing method.

In some embodiments/aspects, the present invention relates to a use of one or more of variants, polypeptides, polynucleotides, nucleic acid constructs, expression vectors, recombinant host cells, spores, transgenic plants, transgenic seeds, transgenic pollen grains, foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent, intermediate detoxifying agent; nutritional supplement, intermediate nutritional supplement; prebiotic, intermediate prebiotic or mixture/s thereof, composition or kit of the present invention for/in one or more of the following: treatment, amelioration, prophylaxis and/or diagnostics of mycotoxicosis, preferably OTA mycotoxicosis; monitoring development of mycotoxicosis and/or assessing the efficacy of a mycotoxicosis prophylaxis and/or therapy, preferably OTA mycotoxicosis prophylaxis and/or therapy; detoxifying and/or altering toxicity of a mycotoxin (e.g. ochratoxin) having Formula I; producing one or more of the following: foodstuff, intermediate foodstuff; fodder, intermediate fodder; feed intermediate feed; additive (e.g., foodstuff-, fodder- or feed additive), intermediate additive (e.g., foodstuff-, fodder- or feed intermediate additive); detoxifying agent, intermediate detoxifying agent; nutritional supplement, intermediate nutritional supplement; prebiotic, intermediate prebiotic; pharmaceutical, veterinary, diagnostic, detoxifying, monitoring and/or screening composition or kit; in method according to present invention; any combination of the above; use as define herein, wherein said use is an in vitro, ex vivo, in vivo and/or manufacturing use.

In some embodiments/aspects, the present invention relates to the use of the variant, polynucleotide, nucleic acid construct, expression vector, recombinant host cell, spore, transgenic plant, transgenic seed, transgenic pollen grain, foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent, intermediate detoxifying agent; nutritional supplement, intermediate nutritional supplement; prebiotic, intermediate prebiotic or mixture/s thereof, composition or kit according to the present invention for the manufacture of a medicament for treatment or prevention of mycotoxicosis.

In some embodiments/aspects, the present invention relates to particular variants of the enzyme of SEQ ID NO: 318 having either of the following combination of mutations: I308V/V355L; F318Y/V355L; S97V/A171P/I184V/S284T/I308V/V355L; S143E/T144A/A171P/I308V/G354A/V355L; H30E/H50N/S97V/G98V/N103D/W104Y/Q119H/T130E/G142A/T144R/Y170F/A171P/I184V/H187N/K188R/M223C/S230T/E239Q/S284T/I308V/V311I/L333I/H339E/A353S/V355L/G356P; I308V/F318Y/V355L; I184V/I308V/V355L; I184V/I308V/F318Y/V355L; H30E/A36C/H50N/L85C/S97V/G98V/S100D/N103Q/W104Y/Y105R/G106P/D107R/F108A/F109Y/D110K/N111D/M112W/M113Q/Q119H/Y122R/Y126W/T130E/G142A/S143E/T144R/Y170F/A171P/G176S/A179C/Q181M/P183S/I84V/H187N/K188R/A192V/V198T/M223C/G226S/V228A/L229I/S230T/L231F/D233N/V235A/E239Q/Q242P/H253E/K258Y/H264Q/D278R/S284T/E287Q/Y300C/A307G/I308V/A309C/H310E/V311A/E313A/K314Q/T317C/F318Y/I322M/A329G/A330M/H332S/L333M/H339E/A353S/G 354A/V355L/G356P/A357P/H358N/N361Q/L389C/G391R; H30E/V32I/A36C/A37G/H50N/D76D/A83S/L85C/S97V/G98V/S100D/N103Q/W104Y/Y105R/G106P/D107R/F108A/F109Y/D110K/N111D/M112W/M113Q/A117G/Q119H/Y122R/Y126W/T130E/V141L/G142A/S143E/T144R/V147I/Y170F/A171P/G176S/A179C/Q181M/P183S/I184V/H187N/K188R/A192V/V198T/E203D/M223C/G226S/V228A/L229I/S230T/L231F/D233N/V235A/E239Q/Q242P/D243E/I249V/H253E/K258Y/H264Q/D278R/S284T/E287Q/Y300C/A307G/308V/A309C/H310E/V311A/E313A/K314Q/T317C/F318Y/I322M/A329G/A330M/H332S/L333M/H339E/A353S/G354A/V355L/G356P/A357P/H358N/S360T/N361Q/L389C/G391R/L425V; H30E/V32I/A36C/A37G/H50N/D76D/A83S/L85C/S97V/G98V/S100D/N103Q/A117G/Q119H/Y122R/Y126W/T130E/V141L/G142A/S143E/T144P/V147I/Y170F/A171P/G176S/A179C/Q181M/P183S/I184V/H187N/K188P/A192V/V198T/E203D/M223C/G226S/V228A/L229I/S230T/L231F/D233N/V235A/E239Q/Q242P/D243E/I249V/H253E/K258Y/H264Q/D278R/S284T/E287Q/Y300C/A307G/I308V/A309C/H310E/V311A/E313A/K314Q/T317C/F318Y/322M/A329G/A330M/H332S/L333M/H339E/A353S/G354A/V355L/G356P/A357P/H358N/S360T/N361Q/L389C/G391P/L425V; Y170F/A171P/G176S/A179C/Q181M/P183S/I184V/H187N/K188R/A192V/V198T/M223C/G226S/V228A/L229I/S230T/L231F/D233N/V235A/E239Q/Q242P/H253E/K258Y/H264Q/D278R/S284T/E287Q/Y300C/A307G/308V/A309C/H310E/V311A/E313A/K314Q/T317C/F318Y/I322M/A329G/A330M/H332S/L333M; or Y170F/A171P/G176S/A179C/Q181M/P183S/I184V/H187N/K188R/A192V/V198T/M223C/G226S/V228A/L229I/S230T/L231F/D233N/V235A/E239Q/Q242P, e.g., using the numbering of SEQ ID NO: 318.

In some embodiments/aspects, the present invention relates to a variant of the enzyme of SEQ ID NO: 318 having the mutation combination I308V/V355L (using the numbering of SEQ ID NO: 318), e.g., which is found to show 189% and 209% of the activity of the non-mutated enzyme of SEQ ID NO: 318 at pH 7.5 and pH 6, respectively.

In some embodiments/aspects, the present invention relates to a variant of the enzyme of SEQ ID NO: 318 having the mutation combination F318Y/V355L, e.g., which is found to show 138% and 197% of the activity of the non-mutated enzyme of SEQ ID NO: 318 at pH 7.5 and pH 6, respectively.

In some embodiments/aspects, the present invention relates to variants of the enzyme of SEQ ID NO: 318 having either of the combination of mutations of H30E/V32I/A36C/A37G/H50N/D76D/A83S/L85C/S97V/G98V/S100D/N103Q/W104Y/Y105R/G106P/D107R/F108A/F109Y/D110K/N111D/M112W/M113Q/A117G/Q119H/Y122R/Y126W/T130E/V141L/G142A/S143E/T144R/V147I/Y170F/A171P/G176S/A179C/Q181M/P183S/I184V/H187N/K188R/A192V/V198T/E203D/M223C/G226S/V228A/L229I/S230T/L231F/D233N/V235A/E239Q/Q242P/D243E/I249V/H253E/K258Y/H264Q/D278R/S284T/E287Q/Y300C/A307G/I308V/A309C/H310E/V311A/E313A/K314Q/T317C/F318Y/I322M/A329G/A330M/H332S/L333M/H339E/A353S/G354A/V355L/G356P/A357P/H358N/S360T/N361Q/L389C/G391R/L425V; H30E/V32I/A36C/A37G/H50N/D76D/A83S/L85C/S97V/G98V/S100D/N103Q/A117G/Q119H/Y122R/Y126W/T130E/V141L/G142A/S143E/T144P/V147I/N170F/A171P/G176S/A179C/Q181M/P183S/I184V/H187N/K188P/A192V/V198T/E203D/M223C/G226S/V228A/L229I/S230T/L231F/D233N/V235A/E239Q/Q242P/D243E/I249V/H253E/K258Y/H264Q/D278R/S284T/E287Q/Y300C/A307G/I308V/A309C/H310E/V311A/E313A/K314Q/T317C/F318Y/I322M/A329G/A330M/H332S/L333M/H339E/A353S/G354A/V355L/G356P/A357P/H358N/S360T/N361Q/L389C/G391P/L425V; Y170F/A171P/G176S/A179C/Q181M/P183S/I184V/H187N/K188R/A192V/V198T/M223C/G226S/V228A/L229I/S230T/L231F/D233N/V235A/E239Q/Q242P/H253E/K258Y/H264Q/D278R/S284T/E287Q/Y300C/A307G/I308V/A309C/H310E/V311A/E313A/K314Q/T317C/F318Y/I322M/A329G/A330M/H332S/L333M; or Y170F/A171P/G176S/A179C/Q181M/P183S/I184V/H187N/K188R/A192V/V198T/M223C/G226S/V228A/L229I/S230T/L231F/D233N/V235A/E239Q/Q242P (e.g., using the numbering of SEQ ID NO: 318), which e.g., are 75%, 80%, 85% or 90%, respectively identical to the amino acid sequence of SEQ ID NO: 1 in a global sequence alignment (i.e. not omitting N- and/or C-terminal amino acids) using the Needleman-Wunsch algorithm as provided by the National Center for Biotechnology Information (“Needleman-Wunsch Global Align Protein Sequences”) using default settings.

In some embodiments/aspects, the present invention relates to variants of the enzyme of SEQ ID NO: 2 having either of the following combination of mutations are produced: I065A/E106G/I272V/Y275V/F318V; I065A/E106G/Y275V/F318V; I065A/Y275V/F318V; R7K/I18L/D26N/I33V/T34S/A40V/K41P/V44I/N45D/L60I/L62I/T63S/I65A/R66K/D77E/L84I/S87S/E106G/I110L/A115G/I116L/N117Q/E118D/I120L/I126V/P132A/A140G/L142I/L146I/G157S/D164E/N173I/A178S/N183I/E192Q/E197D/T202S/E204D/R231K/R 239K/I268A/Q269I/I272V/Y275V/V296L/I300L/K301J/R304K/V308I/L311I/A312T/F318V/D323E/S343T/A355S/S356A/L359I/L361I/T363S/I365L/A372G/I374L/V375I/L376I/N378D/N384D/I385V/D387E/I388V/A389S/R390K/V391I/I395L/V400L/N405D/L408I; or V44I/N45D/L60I/L62I/T63S/I65A/R66K/D77E/L84I/S87S/E106G/I110L/A115G/I16L/N117Q/E118D/I120L/I126V/P132A/A140G/L142I/L146I/G157S/D164E/V173I/A178S/V183I/E192Q/E197D/T202S/E204D/R231K/R239K/I268A/Q269I/I272V/Y275V/V296L/I 300L/K301RR304K/V308I/L311I/A312T/F318V/D323E/S343T/A355S/S356A/L359I/L361I/T363S/I365L/A372G/I374L/V375I/L376I/N378D/N384D/I385V/D387E (e.g., using the numbering of SEQ ID NO: 2), which e.g., are 98.8%, 99.0%, 99.3%, 81.4% or 85.0% identical to the enzyme of SEQ ID NO: 2, respectively.

In some embodiments/aspects, the present invention relates to variants of the enzyme of SEQ ID NO: 22 having either of the following combination of mutations are produced by the inventors: V5I/D38E/N40D/L66I/I74L/A83T/K131R/S153T/D176E/R195K/S202T/A208V/S245T/S261T/T267S/K274R/E305D/L335V/L360I/D379N; or Q2A/Y3F/V5I/I7L/V12I/T14N/S27N/S32T/D38E/N40D/L51F/L66I/G69A/I74L/K78R/A83T/L84/E91D/E106D/K113A/R114K/N117K/K125R/I126V/A129G/K131R/F142L/S143T/S153T/G162A/D176E/S178T/D179E/I183L/R195K/D197E/S202T/F203Y/R207K/A 208V/I209L/A216V/E235D/A236G/S245T/K248Q/E249D/D250E/L256I/I260L/S261T/L262I/T267S/I271V/K274R/E294D/N295Q/L296I/L300V/R301K/E305D/G313A/T320S/I327L/G328A/L335V/S344T/A357S/L360I/L362I/L366I/I367L/D372E/I375L/V377I/D379N/N385D/V389I/Q398D/L401I/D405E/G406A (e.g., using the numbering of SEQ ID NO: 22), wherein e.g., the variants are 95.15% or 80.10% identical to the enzyme of SEQ ID NO: 22, respectively.

In some embodiments/aspects, the present invention relates to novel polypeptides and variants thereof as described herein, exhibiting altered properties, in particular altered thermostability pattern (relative to the parent and/or other known OTA hydrolyzing enzymes) and/or enhanced hydrolyzing activity as disclosed herein, which are advantageous with respect to applications of the polypeptides and variants thereof, in particular, in industrial and/or manufacturing methods as disclosed herein.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

All publications and patents cited throughout the text of this specification (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

The invention is also characterized by the following items:
1. A polypeptide capable of detoxifying (e.g., modifying or hydrolyzing) at least one mycotoxin, preferably an ochratoxin having Formula I (e.g., Ochratoxin A, Ochratoxin B and/or Ochratoxin C):

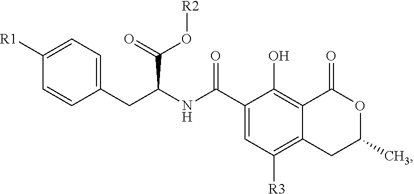

wherein R1 is selected from the group consisting of H and OH, R2 is selected from the group consisting of H and CH2-CH3 and R3 is selected from the group consisting of H and Cl; preferably wherein said polypeptide having a peptidase activity EC 3.4.13.X, wherein X is selected from the group consisting of: 9, 4, 5, 7, 12, 17, 18, 19, 20, 21, 22, 23; wherein said polypeptide is one or more of the following:
   a) a polypeptide having at least 58% sequence identity (e.g. at least 59%, 60%, 61%, 62%, 63%, 64%, 65%, 66%, 67%, 68%, 69%, 70%, 71%, 72%, 73%, 74%, 80%, 81%, 82%, 83%, 84%, 85%, 86%, 87%, 88%, 89%, 90%, 91%, 92%, 93%, 94%, 95%, 96%, 97%, 98%, 99% or 100%) to the amino acid sequence selected from the group consisting of: SEQ ID NOs: 1, 2, 16-21, 22, 23-34, 317-318, 326-335, 336-337, 348-349, 368-370;
   b) a variant of the polypeptide of (a), wherein said variant comprising a substitution, deletion, and/or insertion at one or more positions (e.g., said variant having one or more conservative or equivalent substitutions (e.g., SEQ ID NOs: 35-316, 319-325, 338-347, 350-359);
   c) a fragment of the polypeptide of (a) or (b), wherein said fragment is capable of detoxifying the mycotoxin having said Formula I;
2. A method (e.g., in vitro, ex vivo, in vivo or manufacturing method) for detoxifying (e.g., modifying or hydrolyzing) a mycotoxin having Formula I (e.g., Ochratoxin A, Ochratoxin B and/or Ochratoxin C):

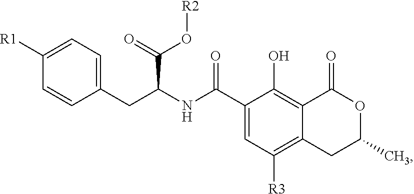

wherein R1 is selected from the group consisting of H and OH, R2 is selected from the group consisting of H and CH₂—CH₃ and R3 is selected from the group consisting of H and Cl; said method comprising: (a) providing:
   (i) one or more polypeptides having at least 70% sequence identity (e.g. at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) to the amino acid sequence selected from the group consisting of: SEQ ID NO: 1, SEQ ID NO: 2, SEQ ID NO: 22, SEQ ID NO: 318, SEQ ID NO: 336, SEQ ID NO: 348, SEQ ID NOs: 16-359, 368-370; wherein said one or more polypeptide are capable of detoxifying (e.g., modifying or hydrolyzing) at least one mycotoxin having said Formula I;
      preferably said polypeptide/s having peptidase activity having EC 3.4.13.X, wherein X is selected from the group consisting of: 9, 4, 5, 7, 12, 17, 18, 19, 20, 21, 22, 23; further preferably said polypeptide/s having a peptidase activity having EC 3.4.13.9; most preferably said one or more polypeptide comprising one or more of the following amino acid sequences (e.g., conservative motifs):
         i′) PGFIDAHVH (SEQ ID NO: 3), preferably said SEQ ID NO: 3 is comprised at amino acid positions corresponding to positions 87-95 of SEQ ID NO: 1 (e.g., using the numbering of SEQ ID NO: 1);
         ii′) PAEQA (SEQ ID NO: 4), preferably said SEQ ID NO: 4 is comprised at amino acid positions corresponding to positions 116-120 of SEQ ID NO: 1 (e.g., using the numbering of SEQ ID NO: 1);
         iii′) GVCNG (SEQ ID NO: 5), preferably said SEQ ID NO: 5 is comprised at amino acid positions corresponding to positions 197-201 of SEQ ID NO: 1 (e.g., using the numbering of SEQ ID NO: 1);
         iv′) CRAAVR (SEQ ID NO: 6), preferably said SEQ ID NO: 6 is comprised at amino acid positions corresponding to positions 205-210 of SEQ ID NO: 1 (e.g., using the numbering of SEQ ID NO: 1);
         v′) IKFMPSGGVLSL (SEQ ID NO: 7), preferably said SEQ ID NO: 7 is comprised at amino acid positions corresponding to positions 220-231 of SEQ ID NO: 1 (e.g., using the numbering of SEQ ID NO: 1);
         vi′) RKVAAH (SEQ ID NO: 8), preferably said SEQ ID NO: 8 is comprised at amino acid positions corresponding to positions 257-262 of SEQ ID NO: 1 (e.g., using the numbering of SEQ ID NO: 1);
         vii′) AGVDSIEHG (SEQ ID NO: 9), preferably said SEQ ID NO: 9 is comprised at amino acid positions corresponding to positions 275-283 of SEQ ID NO: 1 (e.g., using the numbering of SEQ ID NO: 1);
         viii′) VMPGLIDAH (SEQ ID NO: 10), preferably said SEQ ID NO: 10 is comprised at amino acid positions corresponding to positions 51-59 of SEQ ID NO: 2 (e.g., using the numbering of SEQ ID NO: 2);
         ix′) AGFTTVRDCG (SEQ ID NO: 11), preferably said SEQ ID NO: 11 is comprised at amino acid positions corresponding to positions 96-105 of SEQ ID NO: 2 (e.g., using the numbering of SEQ ID NO: 2);
         x′) QTFGH (SEQ ID NO: 12), preferably said SEQ ID NO: 12 is comprised at amino acid positions corresponding to positions 135-139 of SEQ ID NO: 2 (e.g., using the numbering of SEQ ID NO: 2);
         xi′) ATGGVLS (SEQ ID NO: 13), preferably said SEQ ID NO: 13 is comprised at amino acid positions corresponding to positions 185-191 of SEQ ID NO: 2 (e.g., using the numbering of SEQ ID NO: 2);
         xii′) YVAAHAHGD (SEQ ID NO: 14), preferably said SEQ ID NO: 14 is comprised at amino acid positions corresponding to positions 219-227 of SEQ ID NO: 2 or 22 (e.g., using the numbering of SEQ ID NO: 2 or 22);
         xiii′) LTPTL (SEQ ID NO: 15), preferably said SEQ ID NO: 15 is comprised at amino acid positions corresponding to positions 262-266 of SEQ ID NO: 2 or 22 (e.g., using the numbering of SEQ ID NO: 2 or 22);
         xiv′) VMPGLID (SEQ ID NO: 360), preferably said SEQ ID NO: 360 is comprised at amino acid positions corresponding to positions 52-58 of SEQ ID NO: 22 (e.g., using the numbering of SEQ ID NO: 22);
         xv′) KGGSL (SEQ ID NO: 361), preferably said SEQ ID NO: 361 is comprised at amino acid positions corresponding to positions 67-71 of SEQ ID NO: 22 (e.g., using the numbering of SEQ ID NO: 22);
         xvi′) LLLAGFTTVRDCG (SEQ ID NO: 362), preferably said SEQ ID NO: 362 is comprised at amino acid positions corresponding to positions 93-105 of SEQ ID NO: 22 (e.g., using the numbering of SEQ ID NO: 22);
         xvii′) QTFGHGE (SEQ ID NO: 363), preferably said SEQ ID NO: 363 is comprised at amino acid positions corresponding to positions 135-141 of SEQ ID NO: 22 (e.g., using the numbering of SEQ ID NO: 22);
         xviii′) FATGGVLSQRD (SEQ ID NO: 364), preferably said SEQ ID NO: 364 is comprised at amino acid positions corresponding to positions 184-194 of SEQ ID NO: 22 (e.g., using the numbering of SEQ ID NO: 22);
         xix′) IVNEA (SEQ ID NO: 365), preferably said SEQ ID NO: 365 is comprised at amino acid positions corresponding to positions 209-213 of SEQ ID NO: 22 (e.g., using the numbering of SEQ ID NO: 22);
         xx′) IGVDEWGL (SEQ ID NO: 366), preferably said SEQ ID NO: 366 is comprised at amino acid positions corresponding to positions 279-286 of SEQ ID NO: 22 (e.g., using the numbering of SEQ ID NO: 22);
         xxi′) GFETGL (SEQ ID NO: 367), preferably said SEQ ID NO: 367 is comprised at amino acid positions corresponding to positions 317-322 of SEQ ID NO: 22 (e.g., using the numbering of SEQ ID NO: 22).
   ii) one or more variants of a parent polypeptide, wherein said variant/s comprising an alteration (e.g., a substitution, deletion and/or insertion) at one or more positions corresponding to positions of the parent polypeptide, wherein said variant/s having at least 70% (e.g. at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%), but less than 100% sequence identity to a parent amino acid sequence selected from the group consisting of: SEQ ID NOs: 1, 2, 16-21, 22, 23-34, 317-318, 326-335, 336-337, 348-349, 368-370; wherein said variant/s are capable of detoxifying (e.g., modifying or hydrolyzing) at least one mycotoxin having said Formula I (e.g., Ochratoxin A, Ochratoxin B and/or Ochratoxin C);
      preferably said variant/s having peptidase activity having EC 3.4.13.X, wherein X is selected from the group consisting of: 9, 4, 5, 7, 12, 17, 18, 19, 20, 21, 22, 23; further preferably said variant/s having a peptidase activity having EC 3.4.13.9; most preferably said variant/s comprising one or more of the following amino acid sequences (e.g., conservative motifs): (i′)-(xxi′) according to (i); further most preferably said parent polypeptide is selected from the group consisting of: SEQ ID NOs: 1, 2, 16-21, 22, 23-34, 317-318, 326-335, 336-337, 348-349, 368-370;
   (iii) one or more polynucleotides, nucleic acid constructs and/or expression vectors encoding and/or capable of expressing one or more polypeptides and/or one or more variants according to (i)-(ii);
   (iv) one or more recombinant host cells, spores, transgenic plants, transgenic seeds and/or transgenic pollen grains comprising (i), (ii) and/or (iii); and/or
   (v) foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent (e.g., solubilized or granulated or pelleted), intermediate detoxifying agent; nutritional supplement, intermediate nutritional supplement; prebiotic, intermediate prebiotic and/or mixture/s thereof comprising (i), (ii), (iii) and/or (iv);
   (b) applying (a) to said mycotoxin having Formula I.
3. A foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent (e.g., solubilized or granulated or pelleted), intermediate detoxifying agent; nutritional supplement, intermediate nutritional supplement; prebiotic, intermediate prebiotic and/or mixture/s thereof, comprising one or more of the following:
   (i) one or more polypeptides having at least 70% sequence identity (e.g. at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) to the amino acid sequence selected from the group consisting of: SEQ ID NO: 1, SEQ ID NO: 2, SEQ ID NO: 22, SEQ ID NO: 318, SEQ ID NO: 336, SEQ ID NO: 348, SEQ ID NOs: 16-359, 368-370; wherein said polypeptide/s are capable of detoxifying (e.g., modifying or hydrolyzing) at least one mycotoxin having said Formula I (e.g., Ochratoxin A, Ochratoxin B and/or Ochratoxin C):

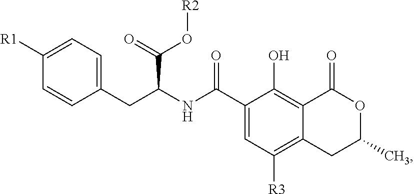

wherein R1 is selected from the group consisting of H and OH, R2 is selected from the group consisting of H and CH₂—CH₃ and R3 is selected from the group consisting of H and Cl;
   preferably said polypeptide/s having a peptidase activity having EC 3.4.13.X, wherein X is selected from the group consisting of: 9, 4, 5, 7, 12, 17, 18, 19, 20, 21, 22, 23; further preferably said polypeptide/s having a peptidase activity having EC 3.4.13.9; most preferably said polypeptide/s comprising one or more of the following amino acid sequences (e.g., conservative motifs):
   i′) PGFIDAHVH (SEQ ID NO: 3), preferably said SEQ ID NO: 3 is comprised at amino acid positions corresponding to positions 87-95 of SEQ ID NO: 1 (e.g., using the numbering of SEQ ID NO: 1);
   ii′) PAEQA (SEQ ID NO: 4), preferably said SEQ ID NO: 4 is comprised at amino acid positions corresponding to positions 116-120 of SEQ ID NO: 1 (e.g., using the numbering of SEQ ID NO: 1);
   iii′) GVCNG (SEQ ID NO: 5), preferably said SEQ ID NO: 5 is comprised at amino acid positions corresponding to positions 197-201 of SEQ ID NO: 1 (e.g., using the numbering of SEQ ID NO: 1);
   iv′) CRAAVR (SEQ ID NO: 6), preferably said SEQ ID NO: 6 is comprised at amino acid positions corresponding to positions 205-210 of SEQ ID NO: 1 (e.g., using the numbering of SEQ ID NO: 1);
   v′) IKFMPSGGVLSL (SEQ ID NO: 7), preferably said SEQ ID NO: 7 is comprised at amino acid positions corresponding to positions 220-231 of SEQ ID NO: 1 (e.g., using the numbering of SEQ ID NO: 1);
   vi′) RKVAAH (SEQ ID NO: 8), preferably said SEQ ID NO: 8 is comprised at amino acid positions corresponding to positions 257-262 of SEQ ID NO: 1 (e.g., using the numbering of SEQ ID NO: 1);
   vii′) AGVDSIEHG (SEQ ID NO: 9), preferably said SEQ ID NO: 9 is comprised at amino acid positions corresponding to positions 275-283 of SEQ ID NO: 1 (e.g., using the numbering of SEQ ID NO: 1);
   viii′) VMPGLIDAH (SEQ ID NO: 10), preferably said SEQ ID NO: 10 is comprised at amino acid positions corresponding to positions 51-59 of SEQ ID NO: 2 (e.g., using the numbering of SEQ ID NO: 2);
   ix′) AGFTTVRDCG (SEQ ID NO: 11), preferably said SEQ ID NO: 11 is comprised at amino acid positions corresponding to positions 96-105 of SEQ ID NO: 2 (e.g., using the numbering of SEQ ID NO: 2);
   x′) QTFGH (SEQ ID NO: 12), preferably said SEQ ID NO: 12 is comprised at amino acid positions corresponding to positions 135-139 of SEQ ID NO: 2 (e.g., using the numbering of SEQ ID NO: 2);
   xi′) ATGGVLS (SEQ ID NO: 13), preferably said SEQ ID NO: 13 is comprised at amino acid positions corresponding to positions 185-191 of SEQ ID NO: 2 (e.g., using the numbering of SEQ ID NO: 2);
   xii′) YVAAHAHGD (SEQ ID NO: 14), preferably said SEQ ID NO: 14 is comprised at amino acid positions corresponding to positions 219-227 of SEQ ID NO: 2 or 22 (e.g., using the numbering of SEQ ID NO: 2 or 22);
   xiii′) LTPTL (SEQ ID NO: 15), preferably said SEQ ID NO: 15 is comprised at amino acid positions corresponding to positions 262-266 of SEQ ID NO: 2 or 22 (e.g., using the numbering of SEQ ID NO: 2 or 22);
   xiv′) VMPGLID (SEQ ID NO: 360), preferably said SEQ ID NO: 360 is comprised at amino acid positions corresponding to positions 52-58 of SEQ ID NO: 22 (e.g., using the numbering of SEQ ID NO: 22);
   xv′) KGGSL (SEQ ID NO: 361), preferably said SEQ ID NO: 361 is comprised at amino acid positions corresponding to positions 67-71 of SEQ ID NO: 22 (e.g., using the numbering of SEQ ID NO: 22);
   xvi′) LLLAGFTTVRDCG (SEQ ID NO: 362), preferably said SEQ ID NO: 362 is comprised at amino acid positions corresponding to positions 93-105 of SEQ ID NO: 22 (e.g., using the numbering of SEQ ID NO: 22);
   xvii′) QTFGHGE (SEQ ID NO: 363), preferably said SEQ ID NO: 363 is comprised at amino acid positions corresponding to positions 135-141 of SEQ ID NO: 22 (e.g., using the numbering of SEQ ID NO: 22);
   xviii′) FATGGVLSQRD (SEQ ID NO: 364), preferably said SEQ ID NO: 364 is comprised at amino acid positions corresponding to positions 184-194 of SEQ ID NO: 22 (e.g., using the numbering of SEQ ID NO: 22);
   xix′) IVNEA (SEQ ID NO: 365), preferably said SEQ ID NO: 365 is comprised at amino acid positions corresponding to positions 209-213 of SEQ ID NO: 22 (e.g., using the numbering of SEQ ID NO: 22);
   xx′) IGVDEWGL (SEQ ID NO: 366), preferably said SEQ ID NO: 366 is comprised at amino acid positions corresponding to positions 279-286 of SEQ ID NO: 22 (e.g., using the numbering of SEQ ID NO: 22);
   xxi′) GFETGL (SEQ ID NO: 367), preferably said SEQ ID NO: 367 is comprised at amino acid positions corresponding to positions 317-322 of SEQ ID NO: 22 (e.g., using the numbering of SEQ ID NO: 22).
ii) one or more variants of a parent polypeptide, wherein said variant/s comprising an alteration (e.g., a substitution, deletion and/or insertion) at one or more positions corresponding to positions of the parent polypeptide, wherein said variant/s having at least 70% (e.g. at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%), but less than 100% sequence identity to a parent amino acid sequence selected from the group consisting of: SEQ ID NOs: 1, 2, 16-21, 22, 23-34, 317-318, 326-335, 336-337, 348-349, 368-370; wherein said variant/s are capable of detoxifying (e.g., modifying or hydrolyzing) at least one mycotoxin having said Formula I (e.g., Ochratoxin A, Ochratoxin B and/or Ochratoxin C);
   preferably said variant/s having a peptidase activity having EC 3.4.13.X, wherein X is selected from the group consisting of: 9, 4, 5, 7, 12, 17, 18, 19, 20, 21, 22, 23; further preferably said variant/s having a peptidase activity having EC 3.4.13.9; most preferably said variant/s comprising one or more of the following amino acid sequences (e.g., conservative motifs): (i′)-(xxi′) according to (i);
(iii) one or more polynucleotides, nucleic acid constructs and/or expression vectors encoding and/or capable of expressing one or more polypeptides and/or one or more variants according to (i)-(ii); and/or
(iv) one or more recombinant host cells, spores, transgenic plants, transgenic seeds and/or transgenic pollen grains comprising (i), (ii) and/or (iii).
4. A variant of a parent polypeptide capable of detoxifying (e.g., modifying or hydrolyzing) at least one mycotoxin having said Formula I (e.g., Ochratoxin A, Ochratoxin B and/or Ochratoxin C):

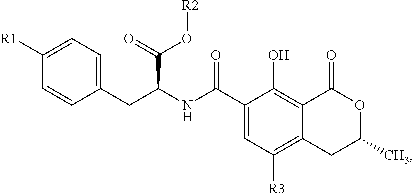

wherein R1 is selected from the group consisting of H and OH, R2 is selected from the group consisting of H and CH₂—CH₃ and R3 is selected from the group consisting of H and Cl; wherein said variant comprising an alteration (e.g., a substitution, deletion and/or insertion) at one or more positions corresponding to positions of the parent polypeptide, wherein said variant/s having at least 70% (e.g. at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%), but less than 100% sequence identity to a parent amino acid sequence selected from the group consisting of: SEQ ID NOs: 1, 2, 16-21, 22, 23-34, 317-318, 326-335, 336-337, 348-349, 368-370;
   preferably said variant having a peptidase activity having EC 3.4.13.X, wherein X is selected from the group consisting of: 9, 4, 5, 7, 12, 17, 18, 19, 20, 21, 22, 23; further preferably said variant having a peptidase activity having EC 3.4.13.9; most preferably, said variant comprising one or more of the following amino acid sequences (e.g., conservative motifs):
   i′) PGFIDAHVH (SEQ ID NO: 3), preferably said SEQ ID NO: 3 is comprised at amino acid positions corresponding to positions 87-95 of SEQ ID NO: 1 (e.g., using the numbering of SEQ ID NO: 1);
   ii′) PAEQA (SEQ ID NO: 4), preferably said SEQ ID NO: 4 is comprised at amino acid positions corresponding to positions 116-120 of SEQ ID NO: 1 (e.g., using the numbering of SEQ ID NO: 1);
   iii′) GVCNG (SEQ ID NO: 5), preferably said SEQ ID NO: 5 is comprised at amino acid positions corresponding to positions 197-201 of SEQ ID NO: 1 (e.g., using the numbering of SEQ ID NO: 1);
   iv′) CRAAVR (SEQ ID NO: 6), preferably said SEQ ID NO: 6 is comprised at amino acid positions corresponding to positions 205-210 of SEQ ID NO: 1 (e.g., using the numbering of SEQ ID NO: 1);
   v) IKFMPSGGVLSL (SEQ ID NO: 7), preferably said SEQ ID NO: 7 is comprised at amino acid positions corresponding to positions 220-231 of SEQ ID NO: 1 (e.g., using the numbering of SEQ ID NO: 1);
   vi′) RKVAAH (SEQ ID NO: 8), preferably said SEQ ID NO: 8 is comprised at amino acid positions corresponding to positions 257-262 of SEQ ID NO: 1 (e.g., using the numbering of SEQ ID NO: 1);
   vii′) AGVDSIEHG (SEQ ID NO: 9), preferably said SEQ ID NO: 9 is comprised at amino acid positions corresponding to positions 275-283 of SEQ ID NO: 1 (e.g., using the numbering of SEQ ID NO: 1);
   viii′) VMPGLIDAH (SEQ ID NO: 10), preferably said SEQ ID NO: 10 is comprised at amino acid positions corresponding to positions 51-59 of SEQ ID NO: 2 (e.g., using the numbering of SEQ ID NO: 2);
   ix′) AGFTTVRDCG (SEQ ID NO: 11), preferably said SEQ ID NO: 11 is comprised at amino acid positions corresponding to positions 96-105 of SEQ ID NO: 2 (e.g., using the numbering of SEQ ID NO: 2);
   x′) QTFGH (SEQ ID NO: 12), preferably said SEQ ID NO: 12 is comprised at amino acid positions corresponding to positions 135-139 of SEQ ID NO: 2 (e.g., using the numbering of SEQ ID NO: 2);
   xi′) ATGGVLS (SEQ ID NO: 13), preferably said SEQ ID NO: 13 is comprised at amino acid positions corresponding to positions 185-191 of SEQ ID NO: 2 (e.g., using the numbering of SEQ ID NO: 2);
   xii′) YVAAHAHGD (SEQ ID NO: 14), preferably said SEQ ID NO: 14 is comprised at amino acid positions corresponding to positions 219-227 of SEQ ID NO: 2 (e.g., using the numbering of SEQ ID NO: 2);
   xiii′) LTPTL (SEQ ID NO: 15), preferably said SEQ ID NO: 15 is comprised at amino acid positions corresponding to positions 262-266 of SEQ ID NO: 2 (e.g., using the numbering of SEQ ID NO: 2);
   xiv′) VMPGLID (SEQ ID NO: 360), preferably said SEQ ID NO: 360 is comprised at amino acid positions corresponding to positions 52-58 of SEQ ID NO: 22 (e.g., using the numbering of SEQ ID NO: 22);
   xv′) KGGSL (SEQ ID NO: 361), preferably said SEQ ID NO: 361 is comprised at amino acid positions corresponding to positions 67-71 of SEQ ID NO: 22 (e.g., using the numbering of SEQ ID NO: 22);
   xvi′) LLLAGFTTVRDCG (SEQ ID NO: 362), preferably said SEQ ID NO: 362 is comprised at amino acid positions corresponding to positions 93-105 of SEQ ID NO: 22 (e.g., using the numbering of SEQ ID NO: 22);
   xvii′) QTFGHGE (SEQ ID NO: 363), preferably said SEQ ID NO: 363 is comprised at amino acid positions corresponding to positions 135-141 of SEQ ID NO: 22 (e.g., using the numbering of SEQ ID NO: 22);
   xviii′) FATGGVLSQRD (SEQ ID NO: 364), preferably said SEQ ID NO: 364 is comprised at amino acid positions corresponding to positions 184-194 of SEQ ID NO: 22 (e.g., using the numbering of SEQ ID NO: 22);
   xix′) IVNEA (SEQ ID NO: 365), preferably said SEQ ID NO: 365 is comprised at amino acid positions corresponding to positions 209-213 of SEQ ID NO: 22 (e.g., using the numbering of SEQ ID NO: 22);
   xx′) IGVDEWGL (SEQ ID NO: 366), preferably said SEQ ID NO: 366 is comprised at amino acid positions corresponding to positions 279-286 of SEQ ID NO: 22 (e.g., using the numbering of SEQ ID NO: 22);
   xxi′) GFETGL (SEQ ID NO: 367), preferably said SEQ ID NO: 367 is comprised at amino acid positions corresponding to positions 317-322 of SEQ ID NO: 22 (e.g., using the numbering of SEQ ID NO: 22);
   further most preferably the number of substitutions is 1-30, preferably, 1-20 and 1-10, such as 1, 2, 3, 4, 5, 6, 7, 8, 9 or 10 substitutions.
5. The method, variant, foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive), detoxifying agent (e.g., solubilized or granulated or pelleted), intermediate detoxifying agent, nutritional supplement, intermediate nutritional supplement; prebiotic, intermediate prebiotic and/or mixture/s thereof according to any one of the preceding items, wherein said polypeptide and/or variant having one or more of the following:
   i) said peptidase activity is a Xaa-Pro dipeptidase EC 3.4.13.9;
   ii) said polypeptide and/or variant having a TIM barrel structure comprising 8 α-helices and 8 parallel β-strands alternating along the polypeptide backbone, wherein preferably said TIM barrel further comprising a phosphate binding site;
   iii) said polypeptide and/or variant having a specific activity of at least 2.9 U/g at pH7.5;
   iv) said polypeptide and/or variant having a specific activity of at least 0.9 U/g at pH6.0;
   v) said polypeptide and/or variant having a T50 value of more than 80° C. (e.g. more than 85° C.);
   vi) said polypeptide is selected from the group consisting of: SEQ ID NOs: 1, 2, 16-21, 22, 23-34, 317-318, 326-335, 336-337, 348-349, 368-370; and/or
   vii) said variant is selected from the group consisting of: SEQ ID NOs: 35-316, 319-325, 338-347, 350-359.
6. The method, variant, foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent (e.g., solubilized or granulated or pelleted), intermediate detoxifying agent; nutritional supplement, intermediate nutritional supplement; prebiotic, intermediate prebiotic and/or mixture/s thereof according to any one of the preceding items, wherein said variant comprising an alteration (e.g., a substitution, deletion and/or insertion) at one or more positions corresponding to positions 65,106,132, 268, 269, 272, 275 and/or 318 of SEQ ID NO: 2, preferably using the numbering of SEQ ID NO: 2.
7. The method, variant, foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent (e.g., solubilized or granulated or pelleted), intermediate detoxifying agent; nutritional supplement, intermediate nutritional supplement; prebiotic, intermediate prebiotic and/or mixture/s thereof according to item 6, wherein said variant comprising one or more of the following substitutions or combinations of substitutions: I65G, I65A, E106G, E106S, P132A, I268A, Q269I, I272V, Y275V; F318V; I272V; Y275F; Y275H; and/or F318V; or equivalent amino acid substitutions thereof.
8. The method, variant, foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent (e.g., solubilized or granulated or pelleted), intermediate detoxifying agent; nutritional supplement, intermediate nutritional supplement; prebiotic, intermediate prebiotic and/or mixture/s thereof according to any one of the preceding items, wherein said variant comprising an alteration (e.g., a substitution, deletion and/or insertion) at one or more positions corresponding to positions 30, 36, 50, 85, 97, 98, 99, 100, 103, 104, 105, 106, 107, 108, 109, 111, 112, 113, 119, 122, 126, 130, 142, 143, 144, 170, 171, 176, 178, 179, 181, 183, 184, 187, 188, 192, 198, 223, 226, 228, 229, 230, 231, 233, 235, 239, 242, 253, 264, 284, 287, 307, 308, 310, 311, 314, 315, 317, 318, 322, 329, 330, 332, 333, 339, 353, 354, 355, 356, 357, 358 or 361 of SEQ ID NOs: 1 or 318, preferably using the numbering of SEQ ID NO: 1 or 318, further preferably using the numbering of SEQ ID NO: 318.
9. The method, variant, foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent (e.g., solubilized or granulated or pelleted), intermediate detoxifying agent; nutritional supplement, intermediate nutritional supplement; prebiotic, intermediate prebiotic and/or mixture/s thereof according to item 8, wherein said variant comprising one or more of the following substitutions or combinations of substitutions: Y300C+L389C; I308V; A171P; T144A; V355L; I308A; G354A; S143E; M223C; A171G; H187N; I184V; H187Q; E239Q; V311I; K314Q; F109A; Y122R; F318Y; A36C+L85C; A375P; M223C; M223A; H264Q; A307G; H178Q; A307G+V355T; T144A; A171P; A171G; M223C; V228A; L229V; L229M; A307S; A307G; I308V; V311T; V355T; V311A; V355A; H264Q; M223C; M223A; A171L; A171G; H178Q; H178L; A307G+V311A; A307G+V311T; A307G+V355T; A307G+A355A; H178S; V228L; V2281; L229I; H264N; H264L; A307V; A307T; I308A; V311L; V311I; G354A; S97D; S97N; S97P; S97T; S97V; G142A; G142P; T144D; T144E; T144P; T144R; A179P; A179S; A179T; A171P+I184V; A171P+I308A; A171P+I308V; A171P+F318Y; A171P+G354A; I184V+I308A; I184V+I308V; I184V+F318Y; I184V+G354A; I308A+F318Y; I308V+F318Y; I308A+G354A; I308V+G354A; F318Y+G354A; A171P+l184V+I308A; A171P+184V+I308V; A171P+184V+F318Y; A171P+184V+G354A; A171P+I308A+F318Y; A171P+I308V+F318Y; A171P+I308A+G354A; A171P+I308V+G354A; A171P+F318Y+G354A; I184V+I308A+F318Y; I184V+I308V+F318Y; I184V+I308A+G354A; I184V+I308V+G354A; I184V+F318Y+G354A; I308A+F318Y+G354A; I308V+F318Y+G354A; Q181M; Q181L; F318L; F318M; A329G; A329V; V355L; V355I; Y170E; Y170F; Y170R; A330L; A330M; A330V; L333I; L333M; L333V; G354L; G354S; G354T; G354V; G356A; G356F; G356P; G356V; G356Y; F109A; P183G; P183S; F108V; F108L; F109V; F109L; M112I; M112W; I184V; I184F; W104F; W104L; W104P; W104Y; I322L; I322M; I322Q; I322S; H187Q; H187N; H310Q; H310N; K314Q; K314E; H332Q; H332V; E287Q; H358Q; H358Y; Q99D; H30E; H30K; H30P; H30R; H50D; H50N; H50P; S100D; N103D; N103L; N103Q; T130E; H187D; H187E; H187G; H187P; K188D; K188E; K188R; G226A; G226P; G226S; S230D; S230P; S230T; D233N; D233Q; D233S; H253A; H253N; S284D; S284H; S284T; H310D; H310E; H332E; H332R; H332S; H339E; H339K; H339N; H339S; A353S; H358A; H358K; H358N; N361E; N361Q; D110K+K188D; D110K+K188E; H187N+K188R; S284D+A329S; S284D+A329T; H310D+E313A; H310E+E313A; V235P; E239Q; A357P; F108Y; N111D; M113Q; Y122R; Y126W; F109Y; Q242P; I315P; T317Y; F318Y; G98S; G98V; Y105N; Y105R; G106P; D107R; D107Y; M112F; M112K; M113D; M113R; Q119E; Q119H; Q119K; Q119M; G176S; A192L; A192M; A192V; V198T; L231F; V235A; V235D; V235F; V235L; L231E+V321T; L229I+I308A; I308A+A329V; I308A+G356F; I308A+G356P; I308A+G356Y; L229I+I308V; I308V+A329V; I308V+G356F; I308V+G356P; I308V+G356Y; G354A+G356V; G354A+G356Y; H310D+V355L; H310E+V355L; V311A+V355L; G354P+V355L; V355L+G356P; I184V+A192L; I184V+A192M; S143E; N361W; I308A+A330L; I308A+A330M; I308A+A330V; I308A+L333I; I308A+L333M; I308V+A330L; I308V+A330M; I308V+A330V; I308V+L333I; I308V+L333M; I308V+V355L; F318Y+V355L; H253E+D278R; K258Y+G391R; A36C+L85C; S1000+T3170; A179C+M223C; Y300C+L389C; A309C+A330C or equivalent amino acid substitutions thereof.
10. The method, variant, foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent (e.g., solubilized or granulated or pelleted), intermediate detoxifying agent; nutritional supplement, intermediate nutritional supplement; prebiotic, intermediate prebiotic and/or mixture/s thereof according to any one of the preceding items, wherein said variant comprising an alteration (e.g., a substitution, deletion and/or insertion) at one or more positions corresponding to positions of SEQ ID NO: 336 or 337 as defined in Table 13 or corresponding to positions of SEQ ID NO: 348 or 349 as defined in Table 14.
11. The method, variant, foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent (e.g., solubilized or granulated or pelleted), intermediate detoxifying agent; nutritional supplement, intermediate nutritional supplement; prebiotic, intermediate prebiotic and/or mixture/s thereof according to item 8, wherein said variant comprising one or more of the substitutions or combinations of substitutions as defined in any of Tables 13-14 herein.
12. A polynucleotide, nucleic acid construct or expression vector encoding and/or capable of expressing one or more variants and/or polypeptides according to any one of the preceding items.
13. A recombinant host cell (e.g., an isolated recombinant host cell), spore, transgenic plant, transgenic seed or transgenic pollen grain comprising one or more of the following: (i) one or more variants and/or polypeptides according to any one of the preceding items; (ii) one or more polynucleotides according to any one of the preceding items; and/or (iii) one or more nucleic acid constructs and/or expression vectors according to any one of the preceding items.
14. A composition or kit comprising one or more of the following: variants, nucleic acid constructs, expression vectors, recombinant host cells, spores, transgenic plants, transgenic seeds and/or transgenic pollen grains, foodstuff, intermediate foodstuff; fodder, intermediate fodder; feed, intermediate feed; additive (e.g., foodstuff-, fodder- or feed additive), intermediate additive (e.g., foodstuff-, fodder- or feed intermediate additive); detoxifying agent (e.g., solubilized or granulated or pelleted), intermediate detoxifying agent; nutritional supplement, intermediate nutritional supplement, prebiotic, intermediate prebiotic and/or mixture/s thereof according to any one of the preceding items and/or one or more polypeptides having at least 70% sequence identity (e.g. at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) to the amino acid sequence selected from the group consisting of: SEQ ID NO: 1, SEQ ID NO: 2, SEQ ID NO: 22, SEQ ID NO: 318, SEQ ID NO: 336, SEQ ID NO: 348, SEQ ID NOs: 17-21, 23-34, 316-359, 368-370; wherein said polypeptide/s are capable of detoxifying (e.g., modifying or hydrolyzing) at least one mycotoxin having said Formula I:

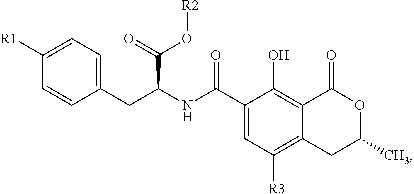

wherein R1 is selected from the group consisting of H and OH, R2 is selected from the group consisting of H and CH₂—CH₃ and R3 is selected from the group consisting of H and Cl;
   preferably said polypeptide/s having a peptidase activity having EC 3.4.13.X, wherein X is selected from the group consisting of: 9, 4, 5, 7, 12, 17, 18, 19, 20, 21, 22, 23; further preferably said polypeptide/s having a peptidase activity having EC 3.4.13.9; most preferably said polypeptide/s comprising one or more of the following amino acid sequences (e.g., conservative motifs):
   i′) PGFIDAHVH (SEQ ID NO: 3), preferably said SEQ ID NO: 3 is comprised at amino acid positions corresponding to positions 87-95 of SEQ ID NO: 1 (e.g., using the numbering of SEQ ID NO: 1);
   ii′) PAEQA (SEQ ID NO: 4), preferably said SEQ ID NO: 4 is comprised at amino acid positions corresponding to positions 116-120 of SEQ ID NO: 1 (e.g., using the numbering of SEQ ID NO: 1);
   iii′) GVCNG (SEQ ID NO: 5), preferably said SEQ ID NO: 5 is comprised at amino acid positions corresponding to positions 197-201 of SEQ ID NO: 1 (e.g., using the numbering of SEQ ID NO: 1);
   iv′) CRAAVR (SEQ ID NO: 6), preferably said SEQ ID NO: 6 is comprised at amino acid positions corresponding to positions 205-210 of SEQ ID NO: 1 (e.g., using the numbering of SEQ ID NO: 1);
   v) IKFMPSGGVLSL (SEQ ID NO: 7), preferably said SEQ ID NO: 7 is comprised at amino acid positions corresponding to positions 220-231 of SEQ ID NO: 1 (e.g., using the numbering of SEQ ID NO: 1);
   vi′) RKVAAH (SEQ ID NO: 8), preferably said SEQ ID NO: 8 is comprised at amino acid positions corresponding to positions 257-262 of SEQ ID NO: 1 (e.g., using the numbering of SEQ ID NO: 1);
   vii′) AGVDSIEHG (SEQ ID NO: 9), preferably said SEQ ID NO: 9 is comprised at amino acid positions corresponding to positions 275-283 of SEQ ID NO: 1 (e.g., using the numbering of SEQ ID NO: 1);
   viii′) VMPGLIDAH (SEQ ID NO: 10), preferably said SEQ ID NO: 10 is comprised at amino acid positions corresponding to positions 51-59 of SEQ ID NO: 2 (e.g., using the numbering of SEQ ID NO: 2);
   ix′) AGFTTVRDCG (SEQ ID NO: 11), preferably said SEQ ID NO: 11 is comprised at amino acid positions corresponding to positions 96-105 of SEQ ID NO: 2 (e.g., using the numbering of SEQ ID NO: 2);
   x′) QTFGH (SEQ ID NO: 12), preferably said SEQ ID NO: 12 is comprised at amino acid positions corresponding to positions 135-139 of SEQ ID NO: 2 (e.g., using the numbering of SEQ ID NO: 2);
   xi′) ATGGVLS (SEQ ID NO: 13), preferably said SEQ ID NO: 13 is comprised at amino acid positions corresponding to positions 185-191 of SEQ ID NO: 2 (e.g., using the numbering of SEQ ID NO: 2);
   xii′) YVAAHAHGD (SEQ ID NO: 14), preferably said SEQ ID NO: 14 is comprised at amino acid positions corresponding to positions 219-227 of SEQ ID NO: 2 (e.g., using the numbering of SEQ ID NO: 2);
   xiii′) LTPTL (SEQ ID NO: 15), preferably said SEQ ID NO: 15 is comprised at amino acid positions corresponding to positions 262-266 of SEQ ID NO: 2 (e.g., using the numbering of SEQ ID NO: 2);
   xiv′) VMPGLID (SEQ ID NO: 360), preferably said SEQ ID NO: 360 is comprised at amino acid positions corresponding to positions 52-58 of SEQ ID NO: 22 (e.g., using the numbering of SEQ ID NO: 22);
   xv′) KGGSL (SEQ ID NO: 361), preferably said SEQ ID NO: 361 is comprised at amino acid positions corresponding to positions 67-71 of SEQ ID NO: 22 (e.g., using the numbering of SEQ ID NO: 22);
   xvi′) LLLAGFTTVRDCG (SEQ ID NO: 362), preferably said SEQ ID NO: 362 is comprised at amino acid positions corresponding to positions 93-105 of SEQ ID NO: 22 (e.g., using the numbering of SEQ ID NO: 22);
   xvii′) QTFGHGE (SEQ ID NO: 363), preferably said SEQ ID NO: 363 is comprised at amino acid positions corresponding to positions 135-141 of SEQ ID NO: 22 (e.g., using the numbering of SEQ ID NO: 22);
   xviii′) FATGGVLSQRD (SEQ ID NO: 364), preferably said SEQ ID NO: 364 is comprised at amino acid positions corresponding to positions 184-194 of SEQ ID NO: 22 (e.g., using the numbering of SEQ ID NO: 22);
   xix′) IVNEA (SEQ ID NO: 365), preferably said SEQ ID NO: 365 is comprised at amino acid positions corresponding to positions 209-213 of SEQ ID NO: 22 (e.g., using the numbering of SEQ ID NO: 22);
   xx′) IGVDEWGL (SEQ ID NO: 366), preferably said SEQ ID NO: 366 is comprised at amino acid positions corresponding to positions 279-286 of SEQ ID NO: 22 (e.g., using the numbering of SEQ ID NO: 22);
   xxi′) GFETGL (SEQ ID NO: 367), preferably said SEQ ID NO: 367 is comprised at amino acid positions corresponding to positions 317-322 of SEQ ID NO: 22 (e.g., using the numbering of SEQ ID NO: 22).
15. The composition or kit according to any one of the preceding items, further comprising one or more of the following (e.g., as shown in Table 2 herein): (i) one or more further polypeptides capable of detoxifying (e.g., modifying and/or hydrolyzing) and/or binding a mycotoxin (e.g. ochratoxin) of Formula I (e.g., OTA, ochratoxin B and/or ochratoxin C), preferably said further one or more polypeptides belonging to the M20 Peptidase aminoacylase 1-like protein 2-like amidohydrolase subfamily (e.g., M20 Peptidase ACY1L2 amidohydrolase subfamily, e.g., having identifier cd05672 according to the Conserved Domain Database (e.g., https://www.ncbi.nlm.nih.gov/Structure/cdd/cddsrv.cgi?uid=cd05672), and/or having aminopeptidase activity (e.g., EC 3.4.11.10), and/or comprise a carboxypeptidase activity (e.g., carboxypeptidase A and/or B activity, e.g., having EC 3.4.17.1 and/or EC 3.4.17.2 respectively) and/or thermolysin activity (e.g., having EC 3.4.24.27); (ii) one or more further polypeptides capable of detoxifying one or more mycotoxins (e.g., ZEN) and/or one or more plant- and/or bacteria-derived toxins (e.g., trichothecene mycotoxin/s such as e.g. deoxynivalenol, nivalenol, neosolaniol, trichotecin, crotocin, roridin A, satratoxin H, diacetoxyscirpenol, HT-2 toxin or T-2 toxin; aflatoxins such as e.g. aflatoxin B1, B2, G1 or G2; fumonisins such as e.g. fumonisin B1, B2, B3 or B4; polypeptide mycotoxins such as e.g. beauvericin or enniatins; zearalenone; citrinin; patulin; ergot alkaloids such as e.g. ergotamine) and/or one or more plant- and/or bacteria-derived toxins (e.g. endotoxin, etc.), in particular said one or more further polypeptides capable of detoxifying one or more further mycotoxins and/or one or more plant- and/or bacteria-derived toxins, e.g., a fumonisin esterase (e.g. as disclosed in WO 2016/134387 A1) and/or a zearalenone lactonase (e.g. as disclosed in WO 2020/025580 A1) and/or an ergopeptine hydrolase (e.g. as disclosed in WO 2014/056006 A1); (iii) one or more organic absorbents (e.g., live/inactivated/lyophilized/dormant/dead whole-yeast or yeast-derived product such as e.g. yeast cell wall, or yeast oligosaccharides such as e.g. mannan) and/or inorganic absorbant (e.g., diatomaceous earth and/or clay mineral such as e.g. kaolins or kaolinites, smectites such as e.g. montmorillonites, illites or chlorites; in particular bentonite); (iv) one or more live, inactivated, lyophilized and/or dormant microorganisms capable of detoxifying one or more further mycotoxin (e.g., trichothecene mycotoxins such as e.g. deoxynivalenol, nivalenol, neosolaniol, trichotecin, crotocin, roridin A, satratoxin H, diacetoxyscirpenol, HT-2 toxin or T-2 toxin; aflatoxins such as e.g. aflatoxin B1, B2, G1 or G2; fumonisins such as e.g. fumonisin B1, B2, B3 or B4; polypeptide mycotoxins such as e.g. beauvericin or enniatins; zearalenone; citrinin; patulin; ergot alkaloids such as e.g. ergotamine) and/or one or more plant- or bacterial-derived toxin (e.g. endotoxin), in particular said microorganism is a member of the Trichosporon or Apiotrichum genus (e.g. as disclosed in WO 03/053161 A1) or of the Coriobacteriaceae family (e.g. as disclosed in EP 3 501 526 A1); (v) one or more plant product (e.g., seaweed, preferably seaweed meal; and/or algae, preferably algae meal; and/or thistle, preferably thistle seeds; and/or glycyrrhiza plant preparation, preferably glycyrrhiza meal and/or glycyrrhiza extract e.g. as disclosed in WO 2018/121881 A1); (vi) one or more flavoring compound (e.g., plant extract e.g. from oregano, thyme, wintergreen, caraway, marjoram, mint, peppermint, anise, orange, lemon, fennel, star anise, clove, cinnamon and/or garlic; and/or essential oil such as e.g. D-limonene, γ-terpinene, p-cymene, 2-carene, linalool oxide, isomenthone, camphor, linalool, terpinen-4-ol, 2-isopropyl-1-methoxy-4-methylbenzene, L-menthol, ethylamine, α-terpineol, β-caryophyllene, D-carvone, methyl salicylate, α-caryophyllene, lavandulyl acetate, caryophyllene oxide, eugenol, thymol and/or carvacrol); (vii) one or more vitamin (e.g. vitamin A, D, E, K, C, B1, B2, B3, B4, B5, B6, B7, B8, B9, B12; in particular vitamin E).
16. A method for producing a foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent, intermediate detoxifying agent; nutritional supplement, intermediate nutritional supplement; prebiotic, intermediate prebiotic and/or mixture/s thereof, said method comprising: a) providing:
   (i) one or more polypeptides having at least 70% sequence identity (e.g. at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) to the amino acid sequence selected from the group consisting of: SEQ ID NO: 1, SEQ ID NO: 2, SEQ ID NO: 22, SEQ ID NO: 318, SEQ ID NO: 336, SEQ ID NO: 348, SEQ ID NOs: 17-21, 23-34, 316-359, 368-370; wherein said polypeptide/s are capable of detoxifying (e.g., modifying or hydrolyzing) at least one mycotoxin having said Formula I (e.g., Ochratoxin A, Ochratoxin B and/or Ochratoxin C):

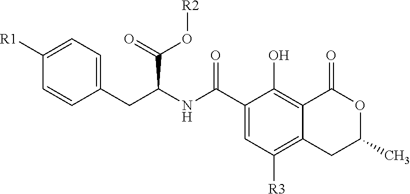

wherein R1 is selected from the group consisting of H and OH, R2 is selected from the group consisting of H and CH₂—CH₃ and R3 is selected from the group consisting of H and Cl;
   preferably said polypeptide/s having a peptidase activity having EC 3.4.13.X, wherein X is selected from the group consisting of: 9, 4, 5, 7, 12, 17, 18, 19, 20, 21, 22, 23; further preferably said polypeptide/s having a peptidase activity having EC 3.4.13.9; most preferably said polypeptide/s comprising one or more of the following amino acid sequences (e.g., conservative motifs):
   i′) PGFIDAHVH (SEQ ID NO: 3), preferably said SEQ ID NO: 3 is comprised at amino acid positions corresponding to positions 87-95 of SEQ ID NO: 1 (e.g., using the numbering of SEQ ID NO: 1);
   ii′) PAEQA (SEQ ID NO: 4), preferably said SEQ ID NO: 4 is comprised at amino acid positions corresponding to positions 116-120 of SEQ ID NO: 1 (e.g., using the numbering of SEQ ID NO: 1);
   iii′) GVCNG (SEQ ID NO: 5), preferably said SEQ ID NO: 5 is comprised at amino acid positions corresponding to positions 197-201 of SEQ ID NO: 1 (e.g., using the numbering of SEQ ID NO: 1);
   iv′) CRAAVR (SEQ ID NO: 6), preferably said SEQ ID NO: 6 is comprised at amino acid positions corresponding to positions 205-210 of SEQ ID NO: 1 (e.g., using the numbering of SEQ ID NO: 1);
   v′) IKFMPSGGVLSL (SEQ ID NO: 7), preferably said SEQ ID NO: 7 is comprised at amino acid positions corresponding to positions 220-231 of SEQ ID NO: 1 (e.g., using the numbering of SEQ ID NO: 1);
   vi′) RKVAAH (SEQ ID NO: 8), preferably said SEQ ID NO: 8 is comprised at amino acid positions corresponding to positions 257-262 of SEQ ID NO: 1 (e.g., using the numbering of SEQ ID NO: 1);
   vii′) AGVDSIEHG (SEQ ID NO: 9), preferably said SEQ ID NO: 9 is comprised at amino acid positions corresponding to positions 275-283 of SEQ ID NO: 1 (e.g., using the numbering of SEQ ID NO: 1);
   viii′) VMPGLIDAH (SEQ ID NO: 10), preferably said SEQ ID NO: 10 is comprised at amino acid positions corresponding to positions 51-59 of SEQ ID NO: 2 (e.g., using the numbering of SEQ ID NO: 2);
   ix′) AGFTTVRDCG (SEQ ID NO: 11), preferably said SEQ ID NO: 11 is comprised at amino acid positions corresponding to positions 96-105 of SEQ ID NO: 2 (e.g., using the numbering of SEQ ID NO: 2);
   x′) QTFGH (SEQ ID NO: 12), preferably said SEQ ID NO: 12 is comprised at amino acid positions corresponding to positions 135-139 of SEQ ID NO: 2 (e.g., using the numbering of SEQ ID NO: 2);
   xi′) ATGGVLS (SEQ ID NO: 13), preferably said SEQ ID NO: 13 is comprised at amino acid positions corresponding to positions 185-191 of SEQ ID NO: 2 (e.g., using the numbering of SEQ ID NO: 2);
   xii′) YVAAHAHGD (SEQ ID NO: 14), preferably said SEQ ID NO: 14 is comprised at amino acid positions corresponding to positions 219-227 of SEQ ID NO: 2 (e.g., using the numbering of SEQ ID NO: 2);
   xiii′) LTPTL (SEQ ID NO: 15), preferably said SEQ ID NO: 15 is comprised at amino acid positions corresponding to positions 262-266 of SEQ ID NO: 2 (e.g., using the numbering of SEQ ID NO: 2);
   xiv′) VMPGLID (SEQ ID NO: 360), preferably said SEQ ID NO: 360 is comprised at amino acid positions corresponding to positions 52-58 of SEQ ID NO: 22 (e.g., using the numbering of SEQ ID NO: 22);
   xv′) KGGSL (SEQ ID NO: 361), preferably said SEQ ID NO: 361 is comprised at amino acid positions corresponding to positions 67-71 of SEQ ID NO: 22 (e.g., using the numbering of SEQ ID NO: 22);
   xvi′) LLLAGFTTVRDCG (SEQ ID NO: 362), preferably said SEQ ID NO: 362 is comprised at amino acid positions corresponding to positions 93-105 of SEQ ID NO: 22 (e.g., using the numbering of SEQ ID NO: 22);
   xvii′) QTFGHGE (SEQ ID NO: 363), preferably said SEQ ID NO: 363 is comprised at amino acid positions corresponding to positions 135-141 of SEQ ID NO: 22 (e.g., using the numbering of SEQ ID NO: 22);
   xviii′) FATGGVLSQRD (SEQ ID NO: 364), preferably said SEQ ID NO: 364 is comprised at amino acid positions corresponding to positions 184-194 of SEQ ID NO: 22 (e.g., using the numbering of SEQ ID NO: 22);
   xix′) IVNEA (SEQ ID NO: 365), preferably said SEQ ID NO: 365 is comprised at amino acid positions corresponding to positions 209-213 of SEQ ID NO: 22 (e.g., using the numbering of SEQ ID NO: 22);
   xx′) IGVDEWGL (SEQ ID NO: 366), preferably said SEQ ID NO: 366 is comprised at amino acid positions corresponding to positions 279-286 of SEQ ID NO: 22 (e.g., using the numbering of SEQ ID NO: 22);
   xxi′) GFETGL (SEQ ID NO: 367), preferably said SEQ ID NO: 367 is comprised at amino acid positions corresponding to positions 317-322 of SEQ ID NO: 22 (e.g., using the numbering of SEQ ID NO: 22),
(ii) variants according to any one of the preceding items;
(iii) one or more polynucleotides according to any one of the preceding items;
(iv) one or more nucleic acid constructs and/or expression vectors according to any one of the preceding items; and/or
(v) one or more recombinant host cells, spores, transgenic plants, transgenic seeds and/or transgenic pollen grains according to any one of the preceding items; preferably said one or more polypeptides are one or more recombinant and/or isolated polypeptides;
b) applying (a) to a nutritive source or material suitable for production of foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent (e.g., solubilized or granulated or pelleted), intermediate detoxifying agent; nutritional supplement, intermediate nutritional supplement; prebiotic, intermediate prebiotic and/or mixture/s thereof.
17. The method according to item 16, wherein (a) is applied to (b) to an enzyme activity concentration in the nutritive source or material suitable for production of foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent (e.g., solubilized or granulated or pelleted), intermediate detoxifying agent; nutritional supplement, intermediate nutritional supplement; prebiotic, intermediate prebiotic and/or mixture/s thereof of at least 5 mU/kg (e.g. at least 10 mU/kg, at least 50 mU/kg, at least 0.1 U/kg, at least 0.2 U/kg, at least 0.29 U/kg, at least 0.5 U/kg, at least 1 U/kg, at least 2 U/kg, at least 2.5 U/kg, at least 2.62 U/kg).
18. A foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent (e.g., solubilized or granulated or pelleted), intermediate detoxifying agent; nutritional supplement, intermediate nutritional supplement; prebiotic, intermediate prebiotic and/or mixture/s thereof produced by the method according to any one of the preceding items.
19. The variant, polynucleotide, nucleic acid construct, expression vector, recombinant host cell, spore, transgenic plant, transgenic seed, transgenic pollen grain, foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent (e.g., solubilized or granulated or pelleted), intermediate detoxifying agent; nutritional supplement, intermediate nutritional supplement; prebiotic, intermediate prebiotic or mixture/s thereof, composition or kit according to any one of the preceding items and/or one or more polypeptides having at least 70% sequence identity (e.g. at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) to the amino acid sequence selected from the group consisting of: SEQ ID NO: 1, SEQ ID NO: 2, SEQ ID NO: 22, SEQ ID NO: 318, SEQ ID NO: 336, SEQ ID NO: 348, SEQ ID NO: 17-21, 23-34, 316-359, 368-370; wherein said polypeptides are capable of detoxifying (e.g., modifying or hydrolyzing) at least one mycotoxin having said Formula I (e.g., Ochratoxin A, Ochratoxin B and/or Ochratoxin C):

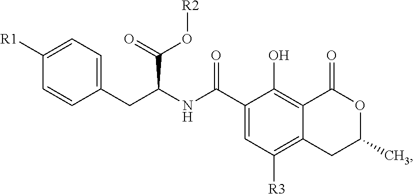

wherein R1 is selected from the group consisting of H and OH, R2 is selected from the group consisting of H and CH₂—CH₃ and R3 is selected from the group consisting of H and Cl;
   preferably said polypeptide/s having a peptidase activity having EC 3.4.13.X, wherein X is selected from the group consisting of: 9, 4, 5, 7, 12, 17, 18, 19, 20, 21, 22, 23; further preferably said polypeptide/s having a peptidase activity having EC 3.4.13.9; most preferably said polypeptide/s comprising one or more of the following amino acid sequences (e.g., conservative motifs):
   i′) PGFIDAHVH (SEQ ID NO: 3), preferably said SEQ ID NO: 3 is comprised at amino acid positions corresponding to positions 87-95 of SEQ ID NO: 1 (e.g., using the numbering of SEQ ID NO: 1);
   ii′) PAEQA (SEQ ID NO: 4), preferably said SEQ ID NO: 4 is comprised at amino acid positions corresponding to positions 116-120 of SEQ ID NO: 1 (e.g., using the numbering of SEQ ID NO: 1);
   iii′) GVCNG (SEQ ID NO: 5), preferably said SEQ ID NO: 5 is comprised at amino acid positions corresponding to positions 197-201 of SEQ ID NO: 1 (e.g., using the numbering of SEQ ID NO: 1);
   iv′) CRAAVR (SEQ ID NO: 6), preferably said SEQ ID NO: 6 is comprised at amino acid positions corresponding to positions 205-210 of SEQ ID NO: 1 (e.g., using the numbering of SEQ ID NO: 1);
   v′) IKFMPSGGVLSL (SEQ ID NO: 7), preferably said SEQ ID NO: 7 is comprised at amino acid positions corresponding to positions 220-231 of SEQ ID NO: 1 (e.g., using the numbering of SEQ ID NO: 1);
   vi′) RKVAAH (SEQ ID NO: 8), preferably said SEQ ID NO: 8 is comprised at amino acid positions corresponding to positions 257-262 of SEQ ID NO: 1 (e.g., using the numbering of SEQ ID NO: 1);
   vii′) AGVDSIEHG (SEQ ID NO: 9), preferably said SEQ ID NO: 9 is comprised at amino acid positions corresponding to positions 275-283 of SEQ ID NO: 1 (e.g., using the numbering of SEQ ID NO: 1);
   viii′) VMPGLIDAH (SEQ ID NO: 10), preferably said SEQ ID NO: 10 is comprised at amino acid positions corresponding to positions 51-59 of SEQ ID NO: 2 (e.g., using the numbering of SEQ ID NO: 2);
   ix′) AGFTTVRDCG (SEQ ID NO: 11), preferably said SEQ ID NO: 11 is comprised at amino acid positions corresponding to positions 96-105 of SEQ ID NO: 2 (e.g., using the numbering of SEQ ID NO: 2);
   x′) QTFGH (SEQ ID NO: 12), preferably said SEQ ID NO: 12 is comprised at amino acid positions corresponding to positions 135-139 of SEQ ID NO: 2 (e.g., using the numbering of SEQ ID NO: 2);
   xi′) ATGGVLS (SEQ ID NO: 13), preferably said SEQ ID NO: 13 is comprised at amino acid positions corresponding to positions 185-191 of SEQ ID NO: 2 (e.g., using the numbering of SEQ ID NO: 2);
   xii′) YVAAHAHGD (SEQ ID NO: 14), preferably said SEQ ID NO: 14 is comprised at amino acid positions corresponding to positions 219-227 of SEQ ID NO: 2 (e.g., using the numbering of SEQ ID NO: 2);
   xiii′) LTPTL (SEQ ID NO: 15), preferably said SEQ ID NO: 15 is comprised at amino acid positions corresponding to positions 262-266 of SEQ ID NO: 2 (e.g., using the numbering of SEQ ID NO: 2);
   xiv′) VMPGLID (SEQ ID NO: 360), preferably said SEQ ID NO: 360 is comprised at amino acid positions corresponding to positions 52-58 of SEQ ID NO: 22 (e.g., using the numbering of SEQ ID NO: 22);
   xv′) KGGSL (SEQ ID NO: 361), preferably said SEQ ID NO: 361 is comprised at amino acid positions corresponding to positions 67-71 of SEQ ID NO: 22 (e.g., using the numbering of SEQ ID NO: 22);
   xvi′) LLLAGFTTVRDCG (SEQ ID NO: 362), preferably said SEQ ID NO: 362 is comprised at amino acid positions corresponding to positions 93-105 of SEQ ID NO: 22 (e.g., using the numbering of SEQ ID NO: 22);
   xvii′) QTFGHGE (SEQ ID NO: 363), preferably said SEQ ID NO: 363 is comprised at amino acid positions corresponding to positions 135-141 of SEQ ID NO: 22 (e.g., using the numbering of SEQ ID NO: 22);
   xviii′) FATGGVLSQRD (SEQ ID NO: 364), preferably said SEQ ID NO: 364 is comprised at amino acid positions corresponding to positions 184-194 of SEQ ID NO: 22 (e.g., using the numbering of SEQ ID NO: 22);
   xix′) IVNEA (SEQ ID NO: 365), preferably said SEQ ID NO: 365 is comprised at amino acid positions corresponding to positions 209-213 of SEQ ID NO: 22 (e.g., using the numbering of SEQ ID NO: 22);
   xx′) IGVDEWGL (SEQ ID NO: 366), preferably said SEQ ID NO: 366 is comprised at amino acid positions corresponding to positions 279-286 of SEQ ID NO: 22 (e.g., using the numbering of SEQ ID NO: 22);
   xxi′) GFETGL (SEQ ID NO: 367), preferably said SEQ ID NO: 367 is comprised at amino acid positions corresponding to positions 317-322 of SEQ ID NO: 22 (e.g., using the numbering of SEQ ID NO: 22),
   for use as a medicament (e.g., for veterinary use) and/or in therapy and/or prophylaxis of a disease.
20. The variant, polynucleotide, nucleic acid construct, expression vector, recombinant host cell, spore, transgenic plant, transgenic seed, transgenic pollen grain, foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent (e.g., solubilized or granulated or pelleted), intermediate detoxifying agent; nutritional supplement, intermediate nutritional supplement; prebiotic, intermediate prebiotic or mixture/s thereof, composition or kit according to any one of the preceding items and/or one or more polypeptides having at least 70% sequence identity (e.g. at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) to the amino acid sequence selected from the group consisting of: SEQ ID NO: 1, SEQ ID NO: 2, SEQ ID NO: 22, SEQ ID NO: 318, SEQ ID NO: 336, SEQ ID NO: 348, SEQ ID NO: 17-21, 23-34, 316-359, 368-370; wherein said polypeptide/s are capable of detoxifying (e.g., modifying or hydrolyzing) at least one mycotoxin having said Formula I (e.g., Ochratoxin A, Ochratoxin B and/or Ochratoxin C):

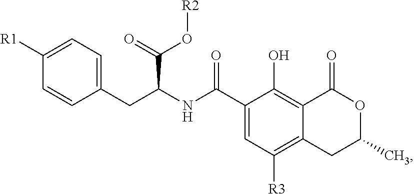

wherein R1 is selected from the group consisting of H and OH, R2 is selected from the group consisting of H and CH₂—CH₃ and R3 is selected from the group consisting of H and Cl;
   preferably said polypeptide/s having a peptidase activity having EC 3.4.13.X, wherein X is selected from the group consisting of: 9, 4, 5, 7, 12, 17, 18, 19, 20, 21, 22, 23; further preferably said polypeptide/s having a peptidase activity having EC 3.4.13.9; most preferably said polypeptide/s comprising one or more of the following amino acid sequences (e.g., conservative motifs):
   i′) PGFIDAHVH (SEQ ID NO: 3), preferably said SEQ ID NO: 3 is comprised at amino acid positions corresponding to positions 87-95 of SEQ ID NO: 1 (e.g., using the numbering of SEQ ID NO: 1);
   ii′) PAEQA (SEQ ID NO: 4), preferably said SEQ ID NO: 4 is comprised at amino acid positions corresponding to positions 116-120 of SEQ ID NO: 1 (e.g., using the numbering of SEQ ID NO: 1);
   iii′) GVCNG (SEQ ID NO: 5), preferably said SEQ ID NO: 5 is comprised at amino acid positions corresponding to positions 197-201 of SEQ ID NO: 1 (e.g., using the numbering of SEQ ID NO: 1);
   iv′) CRAAVR (SEQ ID NO: 6), preferably said SEQ ID NO: 6 is comprised at amino acid positions corresponding to positions 205-210 of SEQ ID NO: 1 (e.g., using the numbering of SEQ ID NO: 1);
   v′) IKFMPSGGVLSL (SEQ ID NO: 7), preferably said SEQ ID NO: 7 is comprised at amino acid positions corresponding to positions 220-231 of SEQ ID NO: 1 (e.g., using the numbering of SEQ ID NO: 1);
   vi′) RKVAAH (SEQ ID NO: 8), preferably said SEQ ID NO: 8 is comprised at amino acid positions corresponding to positions 257-262 of SEQ ID NO: 1 (e.g., using the numbering of SEQ ID NO: 1);
   vii′) AGVDSIEHG (SEQ ID NO: 9), preferably said SEQ ID NO: 9 is comprised at amino acid positions corresponding to positions 275-283 of SEQ ID NO: 1 (e.g., using the numbering of SEQ ID NO: 1);
   viii′) VMPGLIDAH (SEQ ID NO: 10), preferably said SEQ ID NO: 10 is comprised at amino acid positions corresponding to positions 51-59 of SEQ ID NO: 2 (e.g., using the numbering of SEQ ID NO: 2);
   ix′) AGFTTVRDCG (SEQ ID NO: 11), preferably said SEQ ID NO: 11 is comprised at amino acid positions corresponding to positions 96-105 of SEQ ID NO: 2 (e.g., using the numbering of SEQ ID NO: 2);
   x′) QTFGH (SEQ ID NO: 12), preferably said SEQ ID NO: 12 is comprised at amino acid positions corresponding to positions 135-139 of SEQ ID NO: 2 (e.g., using the numbering of SEQ ID NO: 2);
   xi′) ATGGVLS (SEQ ID NO: 13), preferably said SEQ ID NO: 13 is comprised at amino acid positions corresponding to positions 185-191 of SEQ ID NO: 2 (e.g., using the numbering of SEQ ID NO: 2);
   xii′) YVAAHAHGD (SEQ ID NO: 14), preferably said SEQ ID NO: 14 is comprised at amino acid positions corresponding to positions 219-227 of SEQ ID NO: 2 (e.g., using the numbering of SEQ ID NO: 2);
   xiii′) LTPTL (SEQ ID NO: 15), preferably said SEQ ID NO: 15 is comprised at amino acid positions corresponding to positions 262-266 of SEQ ID NO: 2 (e.g., using the numbering of SEQ ID NO: 2);
   xiv′) VMPGLID (SEQ ID NO: 360), preferably said SEQ ID NO: 360 is comprised at amino acid positions corresponding to positions 52-58 of SEQ ID NO: 22 (e.g., using the numbering of SEQ ID NO: 22);
   xv′) KGGSL (SEQ ID NO: 361), preferably said SEQ ID NO: 361 is comprised at amino acid positions corresponding to positions 67-71 of SEQ ID NO: 22 (e.g., using the numbering of SEQ ID NO: 22);
   xvi′) LLLAGFTTVRDCG (SEQ ID NO: 362), preferably said SEQ ID NO: 362 is comprised at amino acid positions corresponding to positions 93-105 of SEQ ID NO: 22 (e.g., using the numbering of SEQ ID NO: 22);
   xvii′) QTFGHGE (SEQ ID NO: 363), preferably said SEQ ID NO: 363 is comprised at amino acid positions corresponding to positions 135-141 of SEQ ID NO: 22 (e.g., using the numbering of SEQ ID NO: 22);
   xviii′) FATGGVLSQRD (SEQ ID NO: 364), preferably said SEQ ID NO: 364 is comprised at amino acid positions corresponding to positions 184-194 of SEQ ID NO: 22 (e.g., using the numbering of SEQ ID NO: 22);
   xix′) IVNEA (SEQ ID NO: 365), preferably said SEQ ID NO: 365 is comprised at amino acid positions corresponding to positions 209-213 of SEQ ID NO: 22 (e.g., using the numbering of SEQ ID NO: 22);
   xx′) IGVDEWGL (SEQ ID NO: 366), preferably said SEQ ID NO: 366 is comprised at amino acid positions corresponding to positions 279-286 of SEQ ID NO: 22 (e.g., using the numbering of SEQ ID NO: 22);
   xxi′) GFETGL (SEQ ID NO: 367), preferably said SEQ ID NO: 367 is comprised at amino acid positions corresponding to positions 317-322 of SEQ ID NO: 22 (e.g., using the numbering of SEQ ID NO: 22),
   for use in one or more of the following methods:
   i) method for treatment, amelioration, prophylaxis and/or diagnostics of mycotoxicosis, preferably an OTA mycotoxicosis;
   ii) method for monitoring development of mycotoxicosis and/or assessing the efficacy of a mycotoxicosis prophylaxis and/or therapy, preferably an OTA mycotoxicosis prophylaxis and/or therapy;
   iii) method for detoxifying and/or altering toxicity of an mycotoxin having Formula I;
   iv) method of producing one or more of the following: foodstuff, intermediate foodstuff; fodder, intermediate fodder; feed intermediate feed; additive (e.g., foodstuff-, fodder- or feed additive), intermediate additive (e.g., foodstuff-, fodder- or feed intermediate additive); detoxifying agent (e.g., solubilized or granulated or pelleted), intermediate detoxifying agent; nutritional supplement, intermediate nutritional supplement; prebiotic, intermediate prebiotic; pharmaceutical, veterinary, diagnostic, detoxifying, monitoring and/or screening composition or kit;
   v) method according to any one of the preceding items;
   vi) any combination of methods according to (i)-(v);
   vii) method of any of (i)-(vi), wherein said method is an in vitro, ex vivo, in vivo and/or manufacturing method.
21. Use of one or more of variants, polynucleotides, nucleic acid constructs, expression vectors, recombinant host cells, spores, transgenic plants, transgenic seeds, transgenic pollen grains, foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent (e.g., solubilized or granulated or pelleted), intermediate detoxifying agent; nutritional supplement, intermediate nutritional supplement; prebiotic, intermediate prebiotic or mixture/s thereof, composition or kit according to any one of the preceding items and/or one or more polypeptides having at least 70% sequence identity (e.g. at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) to the amino acid sequence selected from the group consisting of: SEQ ID NO: 1, SEQ ID NO: 2, SEQ ID NO: 22, SEQ ID NO: 318, SEQ ID NO: 336, SEQ ID NO: 348, SEQ ID NOs: 17-21, 23-34, 316-359, 368-370; wherein said polypeptide/s are capable of detoxifying (e.g., modifying or hydrolyzing) at least one mycotoxin having said Formula I (e.g., Ochratoxin A, Ochratoxin B and/or Ochratoxin C):

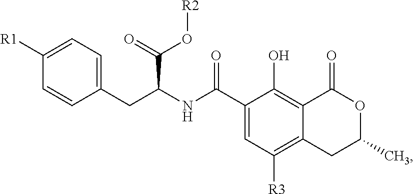

wherein R1 is selected from the group consisting of H and OH, R2 is selected from the group consisting of H and CH₂—CH₃ and R3 is selected from the group consisting of H and Cl; preferably said polypeptide/s having a peptidase activity having EC 3.4.13.X, wherein X is selected from the group consisting of: 9, 4, 5, 7, 12, 17, 18, 19, 20, 21, 22, 23; further preferably said polypeptide/s having a peptidase activity having EC 3.4.13.9; most preferably said polypeptide/s comprising one or more of the following amino acid sequences (e.g., conservative motifs):
   i′) PGFIDAHVH (SEQ ID NO: 3), preferably said SEQ ID NO: 3 is comprised at amino acid positions corresponding to positions 87-95 of SEQ ID NO: 1 (e.g., using the numbering of SEQ ID NO: 1);
   ii′) PAEQA (SEQ ID NO: 4), preferably said SEQ ID NO: 4 is comprised at amino acid positions corresponding to positions 116-120 of SEQ ID NO: 1 (e.g., using the numbering of SEQ ID NO: 1);
   iii′) GVCNG (SEQ ID NO: 5), preferably said SEQ ID NO: 5 is comprised at amino acid positions corresponding to positions 197-201 of SEQ ID NO: 1 (e.g., using the numbering of SEQ ID NO: 1);
   iv′) CRAAVR (SEQ ID NO: 6), preferably said SEQ ID NO: 6 is comprised at amino acid positions corresponding to positions 205-210 of SEQ ID NO: 1 (e.g., using the numbering of SEQ ID NO: 1);
   v′) IKFMPSGGVLSL (SEQ ID NO: 7), preferably said SEQ ID NO: 7 is comprised at amino acid positions corresponding to positions 220-231 of SEQ ID NO: 1 (e.g., using the numbering of SEQ ID NO: 1);
   vi′) RKVAAH (SEQ ID NO: 8), preferably said SEQ ID NO: 8 is comprised at amino acid positions corresponding to positions 257-262 of SEQ ID NO: 1 (e.g., using the numbering of SEQ ID NO: 1);
   vii′) AGVDSIEHG (SEQ ID NO: 9), preferably said SEQ ID NO: 9 is comprised at amino acid positions corresponding to positions 275-283 of SEQ ID NO: 1 (e.g., using the numbering of SEQ ID NO: 1);
   viii′) VMPGLIDAH (SEQ ID NO: 10), preferably said SEQ ID NO: 10 is comprised at amino acid positions corresponding to positions 51-59 of SEQ ID NO: 2 (e.g., using the numbering of SEQ ID NO: 2);
   ix′) AGFTTVRDCG (SEQ ID NO: 11), preferably said SEQ ID NO: 11 is comprised at amino acid positions corresponding to positions 96-105 of SEQ ID NO: 2 (e.g., using the numbering of SEQ ID NO: 2);
   x′) QTFGH (SEQ ID NO: 12), preferably said SEQ ID NO: 12 is comprised at amino acid positions corresponding to positions 135-139 of SEQ ID NO: 2 (e.g., using the numbering of SEQ ID NO: 2);
   xi′) ATGGVLS (SEQ ID NO: 13), preferably said SEQ ID NO: 13 is comprised at amino acid positions corresponding to positions 185-191 of SEQ ID NO: 2 (e.g., using the numbering of SEQ ID NO: 2);
   xii′) YVAAHAHGD (SEQ ID NO: 14), preferably said SEQ ID NO: 14 is comprised at amino acid positions corresponding to positions 219-227 of SEQ ID NO: 2 (e.g., using the numbering of SEQ ID NO: 2);
   xiii′) LTPTL (SEQ ID NO: 15), preferably said SEQ ID NO: 15 is comprised at amino acid positions corresponding to positions 262-266 of SEQ ID NO: 2 (e.g., using the numbering of SEQ ID NO: 2);
   xiv′) VMPGLID (SEQ ID NO: 360), preferably said SEQ ID NO: 360 is comprised at amino acid positions corresponding to positions 52-58 of SEQ ID NO: 22 (e.g., using the numbering of SEQ ID NO: 22);
   xv′) KGGSL (SEQ ID NO: 361), preferably said SEQ ID NO: 361 is comprised at amino acid positions corresponding to positions 67-71 of SEQ ID NO: 22 (e.g., using the numbering of SEQ ID NO: 22);
   xvi′) LLLAGFTTVRDCG (SEQ ID NO: 362), preferably said SEQ ID NO: 362 is comprised at amino acid positions corresponding to positions 93-105 of SEQ ID NO: 22 (e.g., using the numbering of SEQ ID NO: 22);
   xvii′) QTFGHGE (SEQ ID NO: 363), preferably said SEQ ID NO: 363 is comprised at amino acid positions corresponding to positions 135-141 of SEQ ID NO: 22 (e.g., using the numbering of SEQ ID NO: 22);
   xviii′) FATGGVLSQRD (SEQ ID NO: 364), preferably said SEQ ID NO: 364 is comprised at amino acid positions corresponding to positions 184-194 of SEQ ID NO: 22 (e.g., using the numbering of SEQ ID NO: 22);
   xix′) IVNEA (SEQ ID NO: 365), preferably said SEQ ID NO: 365 is comprised at amino acid positions corresponding to positions 209-213 of SEQ ID NO: 22 (e.g., using the numbering of SEQ ID NO: 22);
   xx′) IGVDEWGL (SEQ ID NO: 366), preferably said SEQ ID NO: 366 is comprised at amino acid positions corresponding to positions 279-286 of SEQ ID NO: 22 (e.g., using the numbering of SEQ ID NO: 22);
   xxi′) GFETGL (SEQ ID NO: 367), preferably said SEQ ID NO: 367 is comprised at amino acid positions corresponding to positions 317-322 of SEQ ID NO: 22 (e.g., using the numbering of SEQ ID NO: 22),
   for/in one or more of the following:
   i) treatment, amelioration, prophylaxis and/or diagnostics of mycotoxicosis, preferably OTA mycotoxicosis;
   ii) monitoring development of mycotoxicosis and/or assessing the efficacy of a mycotoxicosis prophylaxis and/or therapy, preferably OTA mycotoxicosis prophylaxis and/or therapy;
   iii) detoxifying and/or altering toxicity of an mycotoxin having Formula I;
   iv) producing one or more of the following: foodstuff, intermediate foodstuff; fodder, intermediate fodder; feed intermediate feed; additive (e.g., foodstuff-, fodder- or feed additive), intermediate additive (e.g., foodstuff-, fodder- or feed intermediate additive); detoxifying agent (e.g., solubilized or granulated or pelleted), intermediate detoxifying agent; nutritional supplement, intermediate nutritional supplement; prebiotic, intermediate prebiotic; pharmaceutical, veterinary, diagnostic, detoxifying, monitoring and/or screening composition or kit;
   v) in method according to any one of the preceding items;
   vi) any combination of according to (i)-(v);
   vii) use of any of (i)-(vi), wherein said use is an in vitro, ex vivo, in vivo use and/or use in a manufacturing method.
22. The method, use, variant, polypeptide, polynucleotide, nucleic acid construct, expression vector, recombinant host cell, spore, transgenic plant, transgenic seed, transgenic pollen grain, foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent (e.g., solubilized or granulated or pelleted), intermediate detoxifying agent; nutritional supplement, intermediate nutritional supplement; prebiotic, intermediate prebiotic or mixture/s thereof, composition or kit according to any one of the preceding items, wherein said SEQ ID NO is SEQ ID NO: 318.
23. The method, use, variant, polypeptide, polynucleotide, nucleic acid construct, expression vector, recombinant host cell, spore, transgenic plant, transgenic seed, transgenic pollen grain, foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent (e.g., solubilized or granulated or pelleted), intermediate detoxifying agent; nutritional supplement, intermediate nutritional supplement; prebiotic, intermediate prebiotic or mixture/s thereof, composition or kit according to any one of the preceding items, wherein said SEQ ID NO is SEQ ID NO: 1.
24. The method, use, variant, polypeptide, polynucleotide, nucleic acid construct, expression vector, recombinant host cell, spore, transgenic plant, transgenic seed, transgenic pollen grain, foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent (e.g., solubilized or granulated or pelleted), intermediate detoxifying agent; nutritional supplement, intermediate nutritional supplement; prebiotic, intermediate prebiotic or mixture/s thereof, composition or kit according to any one of the preceding items, wherein said SEQ ID NO: is SEQ ID NO: 2.
25. The method, use, variant, polypeptide, polynucleotide, nucleic acid construct, expression vector, recombinant host cell, spore, transgenic plant, transgenic seed, transgenic pollen grain, foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent (e.g., solubilized or granulated or pelleted), intermediate detoxifying agent; nutritional supplement, intermediate nutritional supplement; prebiotic, intermediate prebiotic or mixture/s thereof, composition or kit according to any one of the preceding items, wherein said SEQ ID NO is SEQ ID NO: 22.
26. The method, use, variant, polypeptide, polynucleotide, nucleic acid construct, expression vector, recombinant host cell, spore, transgenic plant, transgenic seed, transgenic pollen grain, foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent (e.g., solubilized or granulated or pelleted), intermediate detoxifying agent; nutritional supplement, intermediate nutritional supplement; prebiotic, intermediate prebiotic or mixture/s thereof, composition or kit according to any one of the preceding items, wherein said SEQ ID NO is SEQ ID NO: 336.
27. The method, use, variant, polypeptide, polynucleotide, nucleic acid construct, expression vector, recombinant host cell, spore, transgenic plant, transgenic seed, transgenic pollen grain, foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent (e.g., solubilized or granulated or pelleted), intermediate detoxifying agent; nutritional supplement, intermediate nutritional supplement; prebiotic, intermediate prebiotic or mixture/s thereof, composition or kit according to any one of the preceding items, wherein said SEQ ID NO is SEQ ID NO: 348.
28. The method, use, variant, polypeptide, polynucleotide, nucleic acid construct, expression vector, recombinant host cell, spore, transgenic plant, transgenic seed, transgenic pollen grain, foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent (e.g., solubilized or granulated or pelleted), intermediate detoxifying agent; nutritional supplement, intermediate nutritional supplement; prebiotic, intermediate prebiotic or mixture/s thereof, composition or kit according to any one of the preceding items, wherein a respective OTA-detoxification activity and/or thermostability and/or OTA-degradation capability before pelleting and/or after pelleting of said method, use, variant, polypeptide, polynucleotide, nucleic acid construct, expression vector, recombinant host cell, spore, transgenic plant, transgenic seed, transgenic pollen grain, foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent (e.g., solubilized or granulated or pelleted), intermediate detoxifying agent; nutritional supplement, intermediate nutritional supplement; prebiotic, intermediate prebiotic or mixture/s thereof, composition or kit according to any one of the preceding items is improved, e.g., compared to a reference enzyme/polypeptide, e.g., as defined as in any one of Tables 6-18 herein.
29. The method, use, variant, polypeptide, polynucleotide, nucleic acid construct, expression vector, recombinant host cell, spore, transgenic plant, transgenic seed, transgenic pollen grain, foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent (e.g., solubilized or granulated or pelleted), intermediate detoxifying agent; nutritional supplement, intermediate nutritional supplement; prebiotic, intermediate prebiotic or mixture/s thereof, composition or kit according to any one of the preceding items, using the numbering of SEQ ID NO: 318.

The invention is further illustrated by the following examples, however, without being limited to the example or by any specific embodiment of the examples.

### EXAMPLES OF THE INVENTION

One skilled in the art would readily appreciate that the present invention is well adapted to carry out the objects and obtain the ends and advantages mentioned, as well as those inherent therein. Further, it will be readily apparent to one skilled in the art that varying substitutions and modifications may be made to the invention disclosed herein without departing from the scope and spirit of the invention. The compositions, methods, procedures, treatments, molecules and specific compounds described herein are presently representative of certain embodiments are exemplary and are not intended as limitations on the scope of the invention. Changes therein and other uses will occur to those skilled in the art which are encompassed within the spirit of the invention are defined by the scope of the claims. The listing or discussion of a previously published document in this specification should not necessarily be taken as an acknowledgement that the document is part of the state of the art or is common general knowledge. The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein. All documents, including patent applications and scientific publications, referred to herein are incorporated herein by reference for all purposes. Other embodiments are within the following claims. In addition, where features or aspects of the invention are described in terms of Markush groups, those skilled in the art will recognize that the invention is also thereby described in terms of any individual member or subgroup of members of the Markush group.

### Example 1: Sequence Analyses

Sequence alignments were performed using common amino acid sequence alignment tools as known to a person having skill in the art, such as the ClustalO tool using default settings, or such as the Needleman-Wunsch algorithm e.g. as provided by the National Center for Biotechnology Information (“Needleman-Wunsch Global Align Protein Sequences”) using default settings.

In Tables 3, 4, 5, 6 and 7 below, exemplary calculated sequence identities of sequences are shown to either SEQ ID NO: 1, 2, 22, 336 and 348 respectively. A person having skill in the art is aware of how to determine the sequence identities between any other amino acid sequences. When the N-terminal amino acids are not truncated by the algorithm but considered in a global alignment (e.g. by using the Needleman-Wunsch algorithm), the calculated identity to SEQ ID NO: 1 is lower, e.g. SEQ ID NO: 316 is 78% identical to SEQ ID NO: 1 when the N-terminal amino acids of SEQ ID NO: 316 are not truncated.

TABLE 3
Sequence identities to SEQ ID NO: 1.

| SEQ ID NO: | % |
|---|---|
| 16 | 83.01 |
| 17 | 92.55 |
| 18 | 91.55 |
| 19 | 94.71 |
| 20 | 96.39 |
| 21 | 95.67 |
| 48 | 99.76 |
| 49 | 99.76 |
| 51 | 99.76 |
| 72 | 99.76 |
| 83 | 99.76 |
| 87 | 99.76 |
| 88 | 99.76 |
| 89 | 99.76 |
| 90 | 99.76 |
| 91 | 99.76 |
| 92 | 99.76 |
| 93 | 99.76 |
| 95 | 99.76 |
| 96 | 99.76 |
| 100 | 99.76 |
| 101 | 99.76 |
| 102 | 99.76 |
| 103 | 99.76 |
| 104 | 99.76 |
| 105 | 99.76 |
| 106 | 99.76 |
| 107 | 99.76 |
| 108 | 99.76 |
| 109 | 99.76 |
| 110 | 99.76 |
| 111 | 99.76 |
| 126 | 99.52 |
| 127 | 99.52 |
| 128 | 99.52 |
| 129 | 99.52 |
| 133 | 99.76 |
| 134 | 99.76 |
| 135 | 99.52 |
| 136 | 99.76 |
| 137 | 99.76 |
| 138 | 99.76 |
| 139 | 99.76 |
| 140 | 99.76 |
| 141 | 99.76 |
| 142 | 99.76 |
| 143 | 99.76 |
| 144 | 99.76 |
| 145 | 99.76 |
| 146 | 99.76 |
| 147 | 99.76 |
| 148 | 99.76 |
| 149 | 99.76 |
| 150 | 99.52 |
| 151 | 99.52 |
| 152 | 99.52 |
| 153 | 99.52 |
| 154 | 99.52 |
| 155 | 99.52 |
| 156 | 99.52 |
| 157 | 99.52 |
| 158 | 99.52 |
| 159 | 99.52 |
| 160 | 99.52 |
| 161 | 99.52 |
| 162 | 99.52 |
| 163 | 99.52 |
| 164 | 99.28 |
| 165 | 99.28 |
| 166 | 99.28 |
| 167 | 99.28 |
| 168 | 99.28 |
| 169 | 99.28 |
| 170 | 99.28 |
| 171 | 99.28 |
| 172 | 99.28 |
| 173 | 99.28 |
| 174 | 99.28 |
| 175 | 99.28 |
| 176 | 99.28 |
| 177 | 99.28 |
| 178 | 99.28 |
| 179 | 99.28 |
| 180 | 99.76 |
| 181 | 99.76 |
| 182 | 99.76 |
| 183 | 99.76 |
| 184 | 99.76 |
| 186 | 99.76 |
| 306 | 99.52 |
| 307 | 99.52 |
| 308 | 99.52 |
| 309 | 99.52 |
| 310 | 99.52 |
| 311 | 99.52 |
| 312 | 99.52 |
| 313 | 99.52 |
| 314 | 99.52 |
| 315 | 99.52 |
| 316 | 81.97 |
| 368 | 95.91 |
| 369 | 91.53 |
| 370 | 83.67 |

TABLE 4
Sequence identities to SEQ ID NO: 2.

| SEQ ID NO: | % |
|---|---|
| 22 | 63.39 |
| 23 | 62.90 |
| 24 | 63.30 |
| 25 | 62.90 |
| 26 | 62.16 |
| 27 | 61.08 |
| 28 | 63.05 |
| 29 | 63.30 |
| 30 | 62.81 |
| 31 | 62.56 |
| 32 | 63.79 |
| 33 | 63.05 |
| 34 | 63.55 |
| 35 | 99.75 |
| 36 | 99.75 |
| 37 | 99.75 |
| 38 | 99.75 |
| 39 | 99.75 |
| 40 | 99.75 |
| 41 | 99.75 |
| 42 | 99.75 |
| 43 | 99.75 |
| 44 | 99.75 |
| 45 | 99.75 |
| 46 | 99.75 |
| 47 | 98.04 |

TABLE 5
Sequence identities to SEQ ID NO: 22.

| SEQ ID NO: | % |
|---|---|
| 23 | 99.51 |
| 24 | 73.04 |
| 25 | 74.45 |
| 26 | 76.83 |
| 27 | 71.15 |
| 28 | 72.44 |
| 29 | 73.22 |
| 30 | 72.55 |
| 31 | 72.30 |
| 32 | 72.79 |
| 33 | 72.79 |
| 34 | 73.28 |

TABLE 6
Sequence identities to SEQ ID NO: 336.

| SEQ ID NO: | % |
|---|---|
| 338 | 98 |
| 339 | 98 |
| 340 | 98 |
| 341 | 95 |
| 342 | 95 |
| 343 | 93 |
| 344 | 93 |
| 345 | 90 |
| 346 | 90 |
| 347 | 90 |

TABLE 7
Sequence identities to SEQ ID NO: 348.

| SEQ ID NO: | % |
|---|---|
| 350 | 98 |
| 351 | 98 |
| 352 | 98 |
| 353 | 95 |
| 354 | 95 |
| 355 | 93 |
| 356 | 93 |
| 357 | 90 |
| 358 | 90 |
| 359 | 90 |

To assign an enzyme to an EC number, the amino acid sequence of the enzyme is used to perform homology modelling by using standard modelling algorithms for protein structure modelling, e.g. SWISS-MODELL (https://swissmodel.expasy.org/interactive; Waterhouse et al., Nucleic Acids Res. 46, W296-W303). Such algorithms provide a modeled structure based on the input sequence and match the input sequence-based modeled structure to an enzyme structure previously published e.g. in the RCSB Protein Data Bank (PDB; https://www.rcsb.ora/). In the case of the sequences of the present invention, an enzyme having the structure as described in the RCSB PDB record having the PDB ID: 2QS8 is identified as matching enzyme structure. This enzyme's activity is described as Xaa-Pro dipeptidase, which can be found to have the EC number 3.4.13.9, e.g. in the ENZYME database by Expasy (https://enzyme.expasy.org/). Thus, the enzymes according to the invention have the enzyme activity of EC 3.4.13.9.

### Example 2: Cloning and Recombinant Production

Genes encoding either of the polypeptide sequences SEQ ID NOs: 1-2, 16-359, 368-370 were synthesized by the commercial providers TWIST Bioscience (https://www.twistbioscience.com/) or GenScript (https://www.genscript.com/) with codons optimized for expression in Escherichia coli. Selected genes were initially designed to further encode a hexahistidine tag C-terminally fused to either of the polypeptide sequences to facilitate later purification. Subsequent polypeptide productions and enzyme characterizations were also performed with untagged enzymes yielding essentially identical results.

The genes were cloned into expression vectors using common tools for expression in E. coli as known in the art. In particular, the T7 promoter system with a lac operator (Dubendorf and Studier 1991, J. Mol. Biol. 219, 45-59) was used to regulate gene expression. An E. coli BL21(DE3) strain was transformed with said expression vectors. A kanamycin resistance marker was used to allow selection of transformants on agar plates containing 50 μg/mL kanamycin.

For recombinant protein production, single transformant colonies were incubated over night in terrific broth (TB) medium (containing 12 g/L tryptone, 24 g/L yeast extract, 5 g/L glycerol, 50 mg/L kanamycin) at 37° C. under shaking to produce a preculture. After incubation, 1 mL of this preculture was added to 50 mL of fresh TB medium and incubation was continued as before until an OD600 of 0.5-0.8 was reached. Subsequently, isopropyl-beta-D-thiogalactoside (IPTG) was added to a final concentration of 400 μM to initiate gene expression. The scale of culturing was adapted as per need (e.g. deep-well plate, shake flask, bioreactor) applying common biotechnological methodology. Gene expression was performed by further incubation for 18-20 h at 26-28° C. while shaking. Thereafter, cells were harvested by centrifugation. The supernatant was discarded and the cell pellet was resuspended in 50 mM Tris-HCl buffer, pH 7.5, prior to cell disruption on ice using an ultrasonication system (QSonica). Crude lysate was cleared by centrifugation (18 min, 21,130×g, 4° C.). The presence of recombinant target enzyme in the soluble fraction of the lysate was verified by SDS-PAGE (Laemmli 1970, Nature 227, 680-685) using Bio-Rad 12% mini-PROTEAN TGX stain-free precast gels. Alternative strategies suitable for achieving recombinant protein production either in further E. coli strains, in other bacterial production hosts (e.g. Bacillus sp. such as B. subtilis or B. amyloliquefaciens, Corynebacterium sp. such as C. glutamicum, Pseudomonas sp. etc.) as well as in other eukaryotic hosts (e.g. CHO cells, yeasts e.g. Saccharomyces cerevisiae, Pichia pastoris etc., other fungal hosts e.g. Aspergillus spp., Trichoderma spp. etc.) are described in the state-of-the-art and well-known to a skilled person.

For further purification and isolation of hexahistidine-tagged recombinant enzyme by affinity chromatography, His GraviTrap TALON columns (GE Healthcare) or His MultiTrap High Performance prepacked disposable 96 well plate (Cytiva) were used. To this end, cleared lysate was loaded onto the column, followed by washing with wash buffer containing 10 mM imidazole, 300 mM NaCl, to remove non-target polypeptides. Recombinant target protein bound to the column was eluted with elution buffer containing 150 mM imidazole, 300 mM NaCl. The imidazole contained in the eluate was removed by washing with 50 mM Tris-HCl buffer, pH 7.5, and concomitant enzyme concentration using VivaSpin 6 centrifugation columns (Sartorius). Purified and concentrated enzyme was again analyzed by SDS-PAGE using the software GelAnalyzer (e.g., 2010a version) or the default software of LabChip XII HT Touch (PerkinElmer) to verify the purity of the preparation and to estimate protein concentration by comparison with bovine serum albumin (BSA) standards of known concentrations.

Upon analysis of produced target polypeptides, two species can be found when a gene is expressed encoding the polypeptide having the amino acid sequence of SEQ ID NO: 318. A first species (SEQ ID NO: 1) lacks the N-terminal amino acids 1-24 of SEQ ID NO: 318, a second species (SEQ ID NO: 317) lacks the N-terminal amino acids 1-21 of SEQ ID NO: 318. Analogously, such processed species can be found in polypeptides closely related to the polypeptide of SEQ ID NO: 318, such as the polypeptides of SEQ ID NOs: 16-21, 48-316. Using common sequence alignment it is possible to determine the processed species of homologous variants of the enzyme of SEQ ID NO: 318. For instance, polypeptides of SEQ ID NOs: 326 and 327 are processed species of the polypeptide of SEQ ID NO: 17, SEQ ID NOs: 328 and 329 of SEQ ID NO: 18, SEQ ID NOs: 330 and 331 of SEQ ID NO: 19, SEQ ID NOs: 332 and 333 of SEQ ID NO: 20, SEQ ID NOs: 334 and 335 of SEQ ID NO: 21. Further, polypeptides of SEQ NOs: 336 and 348 are mature, processed species of the polypeptides SEQ ID NOs: 337 and 349, respectively. Also, by applying common methods of molecular biology it is possible to replace the N-terminal amino acids with alternative polypeptides that may also be cleaved by the expression host, such as e.g. either of the leader peptides of lamB, ompA, phoA, torT, dsbA, pac, pelB (SEQ ID NOs: 319-325, respectively), thus also allowing production of active enzyme variants.

### Example 3: Determination of Specific Activities

To determine the specific activity of recombinant target polypeptides produced e.g. as described in Example 2 above in units per liter (U/L), ochratoxin A (OTA) detoxification activity assays were performed. One unit is defined as the amount of enzyme necessary for degradation of 1 μmol or OTA in 1 min when using a starting OTA concentration of 0.495 NM.

OTA detoxification activity assays were set up in a final volume of the reaction mixture of 200 NL, containing the recombinant enzyme preparation (preferably diluted to yield a volumetric activity of 1-10 mU/L), and 200 ng/mL OTA in 100 mM sodium phosphate buffer.

Specific activities were determined at pH 6.0 and pH 7.5. As soon as all components of the reaction mixture were mixed, OTA degradation was followed at 37° C. on a fluorescence spectrophotometer (BioTek Synergy H1 MFD Multimode microplate reader) by following the reduction in fluorescence at ex/em 390 nm/450 nm. For work performed at pH 6.0 and 7.5, a 100 mM sodium phosphate buffer was used.

For calculation of the specific activity, the initial linear part of the recorded fluorescence reduction curve was used. In addition to the fluorescence-based method described above, OTA degradation was also determined by using the HPLC-MS method as described by Dellafiora et al. 2020, Toxins 12, 258, essentially confirming the fluorescence-based results. Genes encoding either of the indicated SEQ ID NOs are expressed as described herein and the specific activities for the obtained polypeptide preparation are exemplarily shown in Table 8. Analogously, enzymatic activities using either ochratoxin B, C or TA as substrate or can be found.

TABLE 8
Specific OTA detoxification activities
in U/g at either pH 6.0 or pH 7.5.

| SEQ ID NO: | pH 6.0 | pH 7.5 |
|---|---|---|
| 1 | 99.00 | 191.89 |
| 2 | 0.61 | 0.73 |
| 17 | 11.65 | 28.44 |
| 18 | 39.30 | 147.14 |
| 19 | 8.72 | 10.91 |
| 20 | 49.73 | 100.52 |
| 21 | 20.53 | 30.87 |
| 22 | 0.66 | 0.79 |
| 368 | 3.70 | 15.10 |
| 369 | 122.50 | 159.80 |

In comparison, an enzyme from Aspergillus niger having the amino acid sequence as laid out in the NCBI GenBank database record CAK41945.1 was found to have a specific OTA detoxification activity of 0.80 U/g and 2.78 U/g at pH 6.0 and pH 7.5, respectively.

Expression of genes encoding polypeptides having selected amino acid substitutions as shown in the SEQ ID NOs indicated below is found to yield further active enzyme variants of the enzyme of SEQ ID NO: 2 as exemplarily shown in Table 7. Additional enzyme variants of the enzyme having the amino acid sequence of SEQ ID NO: 318 being expressed comprise either of the following mutations of H30Q, H30N, H30D, S97A, S97L, N103E, N103S, N103H, F109W, N111E, N111Q, N111S, N111T, M112L, M112V, M112F, M112Y, Y122K, Y126F, T130D, T130Q, T130N, T144G, T144S, T144K, I184A, I184L, I184M, I184Y, I184W, K188N, K188Q, L229V, L229M, S230V, S230A, S230N, Q242A, Q242G, K258W, K258F, S284A, S284V, S284N, E287N, E287H, I308G, I308L, H310S, H310T, V311M, K314N, T317W, T317F, F318W, F3181, A330S, A330T, V355M, G391K, or combinations thereof, using the numbering of SEQ ID NO: 318. For example, particular variants of the enzyme of SEQ ID NO: 318 have either of the following combination of mutations: I308V/V355L; F318Y/V355L; S97V/A171P/I184V/S284T/I308V/V355L; S143E/T144A/A171P/I308V/G354A/V355L; H30E/H50N/S97V/G98V/N103D/W104Y/Q119H/T130E/G142A/T144R/Y170F/A171P/I184V/H187N/K188P/M223C/S230T/E239Q/S284T/I308V/V311I/L333I/H339E/A353S/V355L/G356P; I308V/F318Y/V355L; I184V/I308V/V355L; I184V/I308V/F318Y/V355L; H30E/A36C/H50N/L85C/S97V/G98V/S100D/N103Q/W104Y/Y105R/G106P/D107R/F108A/F109Y/D110K/N111D/M112W/MI113Q/Q119H/Y122R/Y126W/T130E/G142A/S143E/T144R/Y170F/A171P/G176S/A179C/Q181M/P183S/I184V/H187N/K188R/A192V/V198T/M223C/G226S/V228A/L229I/S230T/L231F/D233N/V235A/E239Q/Q242P/H253E/K258Y/H264Q/D278R/S284T/E287Q/Y300C/A307G/I308V/A309C/H310E/V311A/E313A/K314Q/T317C/F318Y/322M/A329G/A330M/H332S/L333M/H339E/A353S/G 354A/V355L/G356P/A357P/H358N/N361Q/L389C/G391R; H30E/V32I/A36C/A37G/H50N/D76D/A83S/L85C/S97V/G98V/S100D/N103Q/W104Y/Y105RG106P/D107RF108A/F109Y/DI110K/N111D/M112W/M113Q/A117G/Q119H/Y122R/Y126W/T130E/V141L/G142A/S143E/T144R/V147I/Y170F/A171P/G176S/A179C/Q181M/P183S/I184V/H187N/K188R/A192V/V198T/E203D/M223C/G226S/V228A/L229I/S230T/L231F/D233N/V235A/E239Q/Q242P/D243E/I249V/H253E/K258Y/H264Q/D278R/S284T/E287Q/Y300C/A307G/I308V/A309C/H310E/V311A/E313A/K314Q/T 317C/F318Y/I322M/A329G/A330M/H332S/L333M/H339E/A353S/G354A/V355L/G356P/A357P/H358N/S360T/N361Q/L389C/G391R/L425V; H30E/V32I/A36C/A37G/H50N/D76D/A83S/L85C/S97V/G98V/S100D/N103Q/A117G/Q119H/Y122R/Y126W/T130E/V141L/G142A/S143E/T144P/V147I/Y170F/A171P/G176S/A179C/Q181M/P183S/I184V/H187N/K188P/A192V/V198T/E203D/M223C/G226S/V228A/L229I/S230T/L231F/D233N/V235A/E239Q/Q242P/D243E/249V/H253E/K258Y/H264Q/D278R/S284T/E287Q/Y300C/A307G/I308V/A309C/H310E/V311A/E313A/K314Q/T317C/F318Y/322M/A329G/A330M/H332S/L333M/H339E/A353S/G354A/V355L/G356P/A357P/H358N/S360T/N361Q/L389C/G391P/L425V; Y170F/A171P/G176S/A179C/Q181M/P183S/I184V/H187N/K188R/A192V/V198T/M223C/G226S/V228A/L229I/S230T/L231F/D233N/V235A/E239Q/Q242P/H253E/K258Y/H264Q/D278R/S284T/E287Q/Y300C/A307G/I308V/A309C/H310E/V311A/E313A/K314Q/T317C/F318Y/I322M/A329G/A330M/H332S/L333M; or Y170F/A171P/G176S/A179C/Q181M/P183S/I184V/H187N/K188R/A192V/V198T/M223C/G226S/V228A/L229I/S230T/L231F/D233N/V235A/E239Q/Q242P using the numbering of SEQ ID NO: 318. The variant of the enzyme of SEQ ID NO: 318 having the mutation combination I308V/V355L (using the numbering of SEQ ID NO: 318) is found to show 189% and 209% of the activity of the non-mutated enzyme of SEQ ID NO: 318 at pH 7.5 and pH 6, respectively. The variant of the enzyme of SEQ ID NO: 318 having the mutation combination F318Y/V355L is found to show 138% and 197% of the activity of the non-mutated enzyme of SEQ ID NO: 318 at pH 7.5 and pH 6, respectively. Notably, variants of the enzyme of SEQ ID NO: 318 having either of the combination of mutations of H30E/V32I/A36C/A37G/H50N/D76D/A83S/L85C/S97V/G98V/S100D/N103Q/W104Y/Y105RG106P/D107RF108A/F109Y/D110K/N111D/M112W/M113Q/A117G/Q119H/Y122R/Y126W/T130E/V141L/G142A/S143E/T144R/V147I/Y170F/A171P/G176S/A179C/Q181M/P183S/I184V/H187N/K188R/A192V/V198T/E203D/M223C/G226S/V228A/L229I/S230T/L231F/D233N/V235A/E239Q/Q242P/D243E/I249V/H253E/K258Y/H264Q/D278R/S284T/E287Q/Y300C/A307G/I308V/A309C/H310E/V311A/E313A/K314Q/T 317C/F318Y/I322M/A329G/A330M/H332S/L333M/H339E/A353S/G354A/V355L/G356P/A357P/H358N/S360T/N361Q/L389C/G391R/L425V; H30E/V32I/A36C/A37G/H50N/D76D/A83S/L85C/S97V/G98V/S100D/N103Q/A117G/Q119H/Y122R/Y126W/T130E/V141L/G142A/S143E/T144P/V147I/N170F/A171P/G176S/A179C/Q181M/P183S/I184V/H187N/K188P/A192V/V198T/E203D/M223C/G226S/V228A/L229I/S230T/L231F/D233N/V235A/E239Q/Q242P/D243E/I249V/H253E/K258Y/H264Q/D278R/S284T/E287Q/Y300C/A307G/I308V/A309C/H310E/V311A/E313A/K314Q/T317C/F318Y/I322M/A329G/A330M/H332S/L333M/H339E/A353S/G354A/V355L/G356P/A357P/H358N/S360T/N361Q/L389C/G391P/L425V; Y170F/A171P/G176S/A179C/Q181M/P183S/I184V/H187N/K188R/A192V/V198T/M223C/G226S/V228A/L229I/S23T/L231F/D233N/V235A/E239Q/Q242P/H253E/K258Y/H264Q/D278R/S284T/E287Q/Y300C/A307G/I308V/A309C/H310E/V311A/E313A/K314Q/T317C/F318Y/I322M/A329G/A330M/H332S/L333M; or Y170F/A171P/G176S/A179C/Q181M/P183S/I184V/H187N/K188R/A192V/V198T/M223C/G226S/V228A/L229I/S230T/L231F/D233N/V235A/E239Q/Q242P (using the numbering of SEQ ID NO: 318) are 75%, 80%, 85% or 90%, respectively identical to the amino acid sequence of SEQ ID NO: 1 in a global sequence alignment (i.e. not omitting N- and/or C-terminal amino acids) using the Needleman-Wunsch algorithm as provided by the National Center for Biotechnology Information (“Needleman-Wunsch Global Align Protein Sequences”) using default settings.

Further exemplary variants of the enzyme of SEQ ID NO: 318 comprising several amino acid substitutions are shown in Table 9 below.

TABLE 9
Variants of the enzyme of SEQ ID NO: 318
comprising the indicated substitution(s)

| SEQ ID NO: | substitutions |
|---|---|
| 48 | F109A |
| 49 | T144A |
| 50 | A171P |
| 51 | A171G |
| 52 | P183G |
| 53 | P183S |
| 54 | V228A |
| 55 | L229V |
| 56 | L229M |
| 57 | A307S |
| 58 | A307G |
| 59 | I308V |
| 60 | V311T |
| 61 | V355T |
| 62 | V235P |
| 63 | E239Q |
| 64 | S143E |
| 65 | N361W |
| 66 | V311A |
| 67 | V355A |
| 68 | A357P |
| 69 | Q181M |
| 70 | Q181L |
| 71 | H264Q |
| 72 | M223C |
| 73 | M223A |
| 74 | A171L |
| 75 | H178Q |
| 76 | H178L |
| 77 | A307G/V311A |
| 78 | A307G/V311T |
| 79 | A307G/V355T |
| 80 | A307G/V355A |
| 81 | V228L |
| 82 | V228L |
| 83 | L229I |
| 84 | H264L |
| 85 | H264L |
| 86 | A307T |
| 87 | I308A |
| 88 | V311L |
| 89 | V311I |
| 90 | G354A |
| 91 | F318L |
| 92 | F318M |
| 93 | A329G |
| 94 | A329V |
| 95 | V355L |
| 96 | V355I |
| 97 | F108V |
| 98 | F108L |
| 99 | F109V |
| 100 | F109L |
| 101 | M112I |
| 102 | M112W |
| 103 | I184V |
| 104 | I184F |
| 105 | H187Q |
| 106 | H187N |
| 107 | H310Q |
| 108 | H310N |
| 109 | K314Q |
| 110 | K314E |
| 111 | H332Q |
| 112 | H332V |
| 113 | E287Q |
| 114 | H358Q |
| 115 | H358Y |
| 116 | Q99D |
| 117 | F108Y |
| 118 | M113Q |
| 119 | Y122R |
| 120 | Y126W |
| 121 | F109Y |
| 122 | Q242P |
| 123 | I315P |
| 124 | T317Y |
| 125 | F318Y |
| 126 | H253E/D278R |
| 127 | K258Y/G391R |
| 128 | A36C/L85C |
| 129 | S100C/T317C |
| 130 | A179C/M223C |
| 131 | Y300C/L389C |
| 132 | A309C/A330C |
| 133 | A307V |
| 134 | N111D |
| 135 | Y300C/L389C |
| 136 | S97D |
| 137 | S97N |
| 138 | S97P |
| 139 | S97T |
| 140 | S97V |
| 141 | G142A |
| 142 | G142P |
| 143 | T144D |
| 144 | T144E |
| 145 | T144P |
| 146 | T144R |
| 147 | A179P |
| 148 | A179S |
| 149 | A179T |
| 150 | A171P/I184V |
| 151 | A171P/I308A |
| 152 | A171P/I308V |
| 153 | A171P/F318Y |
| 154 | A171P/G354A |
| 155 | I184B/I308A |
| 156 | I184V/I308V |
| 157 | I184V/F318Y |
| 158 | I184V/G354A |
| 159 | I308A/F318Y |
| 160 | I308V/F318Y |
| 161 | I308A/G354A |
| 162 | I308V/G354A |
| 163 | F318Y/G354A |
| 164 | A171P/I184V/I308A |
| 165 | A171P/I184V/I308V |
| 166 | A171P/I184V/F318Y |
| 167 | A171P/I184V/G354A |
| 168 | A171P/I308A/F318Y |
| 169 | A171P/I308V/F318Y |
| 170 | A171P/I308A/G354A |
| 171 | A171P/I308V/G354A |
| 172 | A171P/F318Y/G354A |
| 173 | I184V/I308A/F318Y |
| 174 | I184V/I308V/F318Y |
| 175 | I184V/I308A/G354A |
| 176 | I184V/I308V/G354A |
| 177 | I184V/F318Y/G354A |
| 178 | I308A/F318Y/G354A |
| 179 | I308V/F318Y/G354A |
| 180 | Y170E |
| 181 | Y170F |
| 182 | Y170R |
| 183 | A330L |
| 184 | A330M |
| 185 | A330V |
| 186 | L333I |
| 187 | L333M |
| 188 | L333V |
| 189 | G354L |
| 190 | G354S |
| 191 | G354T |
| 192 | G354V |
| 193 | G356A |
| 194 | G356F |
| 195 | G356P |
| 196 | G356V |
| 197 | G356Y |
| 198 | W104F |
| 199 | W104L |
| 200 | W104P |
| 201 | W104Y |
| 202 | I322L |
| 203 | I322M |
| 204 | I322Q |
| 205 | I322S |
| 206 | H30E |
| 207 | H30K |
| 208 | H30P |
| 209 | H30R |
| 210 | H50D |
| 211 | H50N |
| 212 | H50P |
| 213 | S100D |
| 214 | N103D |
| 215 | N103L |
| 216 | N103Q |
| 217 | T130E |
| 218 | H187D |
| 219 | H187E |
| 220 | H187G |
| 221 | H187P |
| 222 | K188D |
| 223 | K188E |
| 224 | K188R |
| 225 | G226A |
| 226 | G226P |
| 227 | G226S |
| 228 | S230D |
| 229 | S230P |
| 230 | S230T |
| 231 | D233N |
| 232 | D233Q |
| 233 | D233S |
| 234 | H253A |
| 235 | H253N |
| 236 | S284D |
| 237 | S284H |
| 238 | S284T |
| 239 | H310D |
| 240 | H310E |
| 241 | H332E |
| 242 | H332R |
| 243 | H332S |
| 244 | H339E |
| 245 | H339K |
| 246 | H339N |
| 247 | H339S |
| 248 | A353S |
| 249 | H358A |
| 250 | H358K |
| 251 | H358N |
| 252 | N361E |
| 253 | N361Q |
| 254 | D110K/K188D |
| 255 | D110K/K188E |
| 256 | H187N/K188R |
| 257 | S284D/A329S |
| 258 | S284D/A329T |
| 259 | H310D/E313A |
| 260 | H310E/E313A |
| 261 | G98S |
| 262 | G98V |
| 263 | Y105N |
| 264 | Y105R |
| 265 | G106P |
| 266 | D107R |
| 267 | D107Y |
| 268 | M112F |
| 270 | M113D |
| 271 | M113R |
| 272 | Q119E |
| 273 | Q119H |
| 274 | Q119K |
| 275 | Q119M |
| 276 | Q176S |
| 277 | A192L |
| 278 | A192L |
| 279 | A192V |
| 280 | V198T |
| 281 | L231F |
| 282 | V235A |
| 283 | V235D |
| 284 | V235F |
| 285 | V235L |
| 286 | L231E/V321T |
| 287 | L229I/I308A |
| 288 | I308A/A329V |
| 289 | I308A/G356F |
| 290 | I308A/G356P |
| 291 | I308A/G356Y |
| 292 | L229I/I308V |
| 293 | I308V/A329V |
| 294 | I308V/G356F |
| 295 | I308V/G356P |
| 296 | I308V/G356Y |
| 297 | G354A/G356V |
| 298 | G354A/G356Y |
| 299 | H310D/V355L |
| 300 | H310E/V355L |
| 301 | V311A/V355L |
| 302 | G354P/V355L |
| 303 | V355L/G356P |
| 304 | I184V/A192L |
| 305 | I184V/A192M |
| 306 | I308A/A330L |
| 307 | I308A/A330M |
| 308 | I308A/A330V |
| 309 | I308A/L333I |
| 310 | I308A/L333M |
| 311 | I308V/A330L |
| 312 | I308V/A330M |
| 313 | I308V/A330V |
| 314 | I308V/L333I |
| 315 | I308V/L333M |
| 316 | H30E/A36C/H50N/L85C/S97V/ |
| | G98V/S100D/N103Q/W104Y/ |
| | Y105R/G106P/D107R/F108A/ |
| | F109Y/D110K/N111D/M112W/ |
| | M113Q/Q119H/Y122R/Y126W/ |
| | T130E/G142A/S143E/T144R/ |
| | Y170F/A171P/G176S/A179C/ |
| | Q181M/P183S/I184V/H187N/ |
| | K188R/A192V/V198T/M223C/ |
| | G226S/V228A/L229I/S230T/ |
| | L231F/D233N/V235A/E239Q/ |
| | Q242P/H253E/K258Y/H264Q/ |
| | D278R/S284T/E287Q/Y300C/ |
| | A307G/I308V/A309C/H310E/ |
| | V311A/E313A/K314Q/T317C/ |
| | F318Y/I322M/A329G/A330M/ |
| | H332S/L333M/H339E/A353S/ |
| | G354A/V355L/G356P/A357P/ |
| | H3358N/N361Q/L389C/G391R |

Further exemplary variants of the enzyme of SEQ ID NO: 2 comprising several amino acid substitutions are shown in Table 10 below.

TABLE 10
Variants of the enzyme of SEQ ID NO: 2 comprising
the indicated substitution(s)

| SEQ ID | |
|---|---|
| NO: | substitutions |
| 35 | I065G |
| 36 | I065A |
| 37 | E106G |
| 38 | E106S |
| 39 | I268A |
| 40 | Q269I |
| 41 | Y275V |
| 42 | F318V |
| 43 | P132A |
| 44 | I272V |
| 45 | Y275F |
| 46 | Y275H |
| 47 | I065A/E106G/I268A/Q269I/Y275V/F318V/P132A/I272V |

In addition and in particular, enzyme variants of the enzyme of SEQ ID NO: 2 having either of the following combination of mutations are produced: I065A/E106G/I272V/Y275V/F318V; I065A/E106G/Y275V/F318V; I065A/Y275V/F318V; R7K/I18L/D26N/I33V/T34S/A40V/K41R/V44I/N45D/L60I/L62I/T63S/I65A/R66K/D77E/L84I/S87S/E106G/I110L/A115G/I116L/N117Q/E118D/I120L/I126V/P132A/A140G/L142I/L146I/G157S/D164E/N173I/A178S/N183I/E192Q/E197D/T202S/E204D/R231K/R239K/I268A/Q269I/I272V/Y275V/V296L/I300L/K301R/R304K/V308I/L311I/A312T/F318V/D323E/S343T/A355S/S356A/L359I/L361I/T363S/I365L/A372G/I374L/V375I/L376I/N378D/N384D/I385V/D387E/I388V/A389S/R390K/V391I/I395L/V400L/N405D/L408I; or V44I/N45D/L60I/L62I/T63S/I65A/R66K/D77E/L84I/S87S/E106G/I110L/A115G/I116L/N117Q/E118D/I120L/I126V/P132A/A140G/L142I/L146I/G157S/D164E/V173I/A178S/V183I/E192Q/E197D/T202S/E204D/R231K/R239K/I268A/Q269I/I272V/Y275V/V296L/I300U/K301R/R304K/V308I/L311I/A312T/F318V/D323E/S343T/A355S/S356A/L359I/L361I/T363S/I365L/A372G/I374L/V375I/L376I/N378D/N384D/I385V/D387E (using the numbering of SEQ ID NO: 2), wherein the variants are 98.8%, 99.0%, 99.3%, 81.4% or 85.0% identical to the enzyme of SEQ ID NO: 2, respectively.

Moreover, variants of the enzyme of SEQ ID NO: 22 having either of the following combination of mutations are produced by the inventors: V5I/D38E/N40D/L66I/I74L/A83T/K131R/S153T/D176E/R195K/S202T/A208V/S245T/S261T/T267S/K274R/E305D/L335V/L360I/D379N; or Q2A/Y3F/V5I/I7L/V12I/T14N/S27N/S32T/D38E/N40D/L51F/L66I/G69A/I74L/K78R/A83T/L84I/E91D/E106D/K113A/R114K/N117K/K125R/I126V/A129G/K131R/F142U/S143T/S153T/G162A/D176E/S178T/D179E/I183L/R195K/D197E/S202T/F203Y/R207K/A 208V/I209L/A216V/E235D/A236G/S245T/K248Q/E249D/D250E/L256I/I260L/S261T/L262I/T267S/I271V/K274R/E294D/N295Q/L296I/L300V/R301K/E305D/G313A/T320S/I327L/G328A/L335V/S344T/A357S/L360I/L362I/L366I/I367L/D372E/I375L/V377I/D379N/N385D/V389I/Q398D/L401I/D405E/G406A (using the numbering of SEQ ID NO: 22), wherein the variants are 95.15% or 80.10% identical to the enzyme of SEQ ID NO: 22, respectively.

TABLE 11
Specific OTA detoxification activities in
% compared to the enzyme of SEQ ID NO: 2.

| SEQ ID NO: | pH 6.0 | pH 7.5 |
|---|---|---|
| 35 | 56.00 | 136.00 |
| 36 | 117.00 | 309.00 |
| 37 | 42.00 | 187.00 |
| 38 | 11.00 | 53.00 |
| 39 | 8.00 | 15.00 |
| 40 | 15.00 | 61.00 |
| 41 | 272.00 | 1551.00 |
| 42 | 131.00 | 595.00 |
| 43 | 46.00 | 19.00 |
| 44 | 84.00 | 84.00 |
| 45 | 168.00 | 138.00 |
| 46 | 82.00 | 66.00 |
| 47 | 85.00 | 267.00 |

Expression of genes encoding polypeptides having selected amino acid substitutions as shown in the SEQ ID NOs indicated in Table 12 below is found to yield further active enzyme variants of the enzyme of SEQ ID NO: 318 as exemplarily shown in Table 12.

TABLE 12
Specific OTA detoxification activities in %
compared to the enzyme of SEQ ID NO: 1.

| SEQ ID | | |
|---|---|---|
| NO: | pH 6.0 | pH 7.5 |
| 48 | 54 | 102 |
| 49 | 76 | 179 |
| 50 | 81 | 257 |
| 51 | 46 | 140 |
| 52 | 16 | 44 |
| 53 | 64 | 100 |
| 54 | 33 | 40 |
| 55 | 27 | 54 |
| 56 | 32 | 87 |
| 57 | 15 | 16 |
| 58 | 6 | 8 |
| 59 | 69 | 333 |
| 60 | 23 | 61 |
| 61 | 15 | 34 |
| 62 | 35 | 26 |
| 63 | 43 | 112 |
| 64 | 36 | 146 |
| 65 | 4 | 10 |
| 66 | 15 | 15 |
| 67 | 51 | 28 |
| 68 | 70 | 67 |
| 69 | 102 | 75 |
| 70 | 80 | 77 |
| 71 | 32 | 17 |
| 72 | 34 | 46 |
| 73 | 20 | 23 |
| 77 | 5 | 3 |
| 80 | 7 | 3 |
| 83 | 63 | 90 |
| 84 | 3 | 5 |
| 85 | 5 | 3 |
| 86 | 4 | 9 |
| 87 | 65 | 166 |
| 88 | 66 | 91 |
| 89 | 43 | 107 |
| 90 | 99 | 162 |
| 91 | 49 | 73 |
| 92 | 37 | 52 |
| 93 | 20 | 17 |
| 95 | 108 | 178 |
| 96 | 42 | 33 |
| 99 | 52 | 58 |
| 100 | 43 | 28 |
| 101 | 82 | 89 |
| 102 | 50 | 89 |
| 103 | 101 | 124 |
| 104 | 102 | 71 |
| 105 | 81 | 120 |
| 106 | 88 | 126 |
| 107 | 54 | 92 |
| 108 | 61 | 96 |
| 109 | 74 | 107 |
| 110 | 33 | 48 |
| 111 | 8 | 52 |
| 113 | 31 | 95 |
| 117 | 40 | 80 |
| 118 | 26 | 22 |
| 119 | 107 | 101 |
| 120 | 85 | 90 |
| 121 | 84 | 78 |
| 122 | 59 | 80 |
| 124 | 87 | 78 |
| 125 | 58 | 94 |
| 126 | 63 | 75 |
| 127 | 78 | 82 |
| 128 | 67 | 73 |
| 129 | 51 | 83 |
| 133 | 23 | 27 |
| 134 | 41 | 52 |
| 135 | 68 | 70 |
| 136 | 64 | 48 |
| 137 | 86 | 93 |
| 138 | 46 | 29 |
| 139 | 141 | 88 |
| 140 | 118 | 120 |
| 141 | 138 | 123 |
| 142 | 34 | 8 |
| 143 | 86 | 84 |
| 144 | 78 | 95 |
| 145 | 101 | 90 |
| 146 | 172 | 112 |
| 148 | 40 | 19 |
| 149 | 30 | 13 |
| 150 | 89 | 62 |
| 151 | 75 | 86 |
| 152 | 65 | 74 |
| 153 | 72 | 77 |
| 154 | 38 | 38 |
| 155 | 116 | 124 |
| 156 | 144 | 109 |
| 157 | 78 | 63 |
| 158 | 157 | 92 |
| 159 | 84 | 93 |
| 160 | 78 | 84 |
| 161 | 132 | 110 |
| 162 | 191 | 124 |
| 163 | 145 | 112 |
| 164 | 29 | 63 |
| 165 | 46 | 71 |
| 166 | 72 | 68 |
| 167 | 54 | 73 |
| 168 | 41 | 62 |
| 169 | 50 | 45 |
| 170 | 49 | 32 |
| 171 | 69 | 37 |
| 172 | 62 | 70 |
| 173 | 27 | 53 |
| 174 | 45 | 59 |
| 175 | 121 | 108 |
| 176 | 112 | 95 |
| 177 | 94 | 90 |
| 178 | 85 | 88 |
| 179 | 49 | 66 |
| 180 | 91 | 83 |
| 181 | 138 | 112 |
| 182 | 98 | 91 |
| 183 | 58 | 55 |
| 184 | 103 | 76 |
| 185 | 32 | 18 |
| 186 | 142 | 115 |
| 187 | 51 | 58 |
| 188 | 84 | 52 |
| 189 | 82 | 22 |
| 190 | 122 | 85 |
| 191 | 21 | 28 |
| 192 | 69 | 30 |
| 193 | 81 | 85 |
| 194 | 78 | 52 |
| 195 | 148 | 100 |
| 196 | 48 | 58 |
| 197 | 59 | 29 |
| 198 | 111 | 84 |
| 199 | 9 | 34 |
| 200 | 18 | 25 |
| 201 | 134 | 121 |
| 202 | 59 | 47 |
| 203 | 100 | 70 |
| 204 | 41 | 24 |
| 205 | 52 | 24 |
| 206 | 168 | 112 |
| 207 | 87 | 72 |
| 208 | 135 | 108 |
| 209 | 143 | 88 |
| 210 | 93 | 102 |
| 211 | 121 | 111 |
| 212 | 46 | 17 |
| 213 | 76 | 39 |
| 214 | 123 | 108 |
| 215 | 27 | 20 |
| 216 | 147 | 115 |
| 217 | 153 | 139 |
| 218 | 104 | 101 |
| 219 | 87 | 88 |
| 220 | 85 | 83 |
| 222 | 8 | 20 |
| 223 | 14 | 16 |
| 224 | 169 | 132 |
| 225 | 24 | 27 |
| 226 | 19 | 22 |
| 227 | 47 | 36 |
| 228 | 23 | 19 |
| 229 | 21 | 15 |
| 230 | 141 | 127 |
| 231 | 20 | 22 |
| 233 | 11 | 17 |
| 234 | 23 | 13 |
| 235 | 56 | 38 |
| 236 | 19 | 15 |
| 237 | 49 | 26 |
| 238 | 151 | 97 |
| 239 | 73 | 76 |
| 240 | 86 | 101 |
| 241 | 18 | 55 |
| 242 | 41 | 54 |
| 243 | 37 | 77 |
| 244 | 127 | 93 |
| 245 | 62 | 59 |
| 246 | 113 | 89 |
| 247 | 80 | 100 |
| 248 | 131 | 91 |
| 249 | 21 | 26 |
| 250 | 17 | 21 |
| 251 | 53 | 33 |
| 252 | 32 | 13 |
| 253 | 93 | 87 |
| 254 | 32 | 15 |
| 256 | 116 | 101 |
| 257 | 34 | 15 |
| 258 | 29 | 25 |
| 259 | 56 | 54 |
| 260 | 83 | 81 |
| 261 | 78 | 95 |
| 262 | 116 | 106 |
| 263 | 19 | 11 |
| 264 | 46 | 44 |
| 265 | 20 | 11 |
| 266 | 11 | 22 |
| 268 | 25 | 39 |
| 271 | 6 | 23 |
| 272 | 121 | 86 |
| 273 | 134 | 76 |
| 274 | 100 | 100 |
| 275 | 71 | 85 |
| 276 | 13 | 30 |
| 277 | 6 | 14 |
| 278 | 32 | 19 |
| 279 | 23 | 36 |
| 280 | 70 | 50 |
| 281 | 90 | 50 |
| 282 | 77 | 41 |
| 283 | 29 | 23 |
| 284 | 48 | 23 |
| 285 | 28 | 21 |
| 286 | 39 | 17 |
| 287 | 151 | 105 |
| 288 | 29 | 31 |
| 289 | 66 | 106 |
| 290 | 160 | 122 |
| 291 | 77 | 79 |
| 292 | 77 | 81 |
| 293 | 16 | 18 |
| 294 | 31 | 42 |
| 295 | 95 | 110 |
| 296 | 42 | 38 |
| 297 | 182 | 106 |
| 298 | 15 | 17 |
| 299 | 156 | 95 |
| 300 | 152 | 103 |
| 301 | 55 | 43 |
| 302 | 26 | 23 |
| 303 | 167 | 110 |
| 304 | 25 | 24 |
| 305 | 36 | 46 |
| 306 | 114 | 74 |
| 307 | 138 | 84 |
| 308 | 171 | 117 |
| 309 | 106 | 48 |
| 310 | 78 | 91 |
| 311 | 89 | 48 |
| 312 | 81 | 64 |
| 313 | 84 | 54 |
| 314 | 71 | 33 |
| 315 | 38 | 56 |
| 316 | 62 | 61 |

Additionally, the enzyme having the amino acid sequence of SEQ ID NO: 336 and other variants of the polypeptide of SEQ ID NO: 337 are found to have a specific OTA detoxification activity of approximately 40 U/g and 22 U/g at pH 7.5 and at pH 6.0, respectively. Exemplary variants of the enzyme of SEQ ID NO: 337 comprising several amino acid substitutions are shown in Table 13 below, using the numbering of SEQ ID NO: 337 and comprising an N-terminal signal peptide analogous to the enzyme of SEQ ID NO: 337.

TABLE 13
Variants of the enzyme of SEQ ID NO: 337
comprising the indicated substitution(s)

| SEQ ID | |
|---|---|
| NO: | substitutions |
| 338 | S148V/S194E/S195A/A222P/V235I/ |
| | G359V/Y369F/M384L/V406L |
| 339 | A222P/V235I/L280I/Y369F/A380V/ |
| | S381V/M3841/G405A/E407P |
| 340 | A222P/V235I/L280I/G359V/Y369F/ |
| | A380V/S381L/G405A/E407F |
| 341 | R83E/P103N/S148V/S149V/K154D/ |
| | W155Y/Q170H/T181E/G193A/S195R/ |
| | Y221F/A222P/V235I/Q238N/R239K/ |
| | M274C/S281T/E290Q/T335S/G359V/ |
| | V372I |
| 342 | W155Y/Q170H/T181E/G193A/S195R/ |
| | Y221F/A222P/V235I/Q238N/R239K/ |
| | M274C/S281T/E290Q/T335S/G359V/ |
| | V372I/M384I/R390E/A404S/V406L/ |
| | E407P |
| 343 | R83E/P103N/S148V/S149V/K154D/W155Y/ |
| | Q170H/T181E/G193A/S195R/Y221F/A222P/ |
| | V235I/Q238N/R239K/M274C/L280I/S281T/ |
| | E290Q/T335S/G359V/Y369F/V372I/S381V/ |
| | M384I/R390E/A404S/V406L/E407P |
| 344 | P103N/A115R/S148V/W155Y/Q170H/H176Y/ |
| | T181E/G193A/S195R/Y221F/A222P/V235I/ |
| | Q238N/R239K/M274C/L280I/S281T/E290Q/ |
| | E294D/T335S/G359V/Y369F/V372I/S381V/ |
| | M384I/R390E/A404S/V406L/E407P |
| 345 | R83E/A89C/P103N/L136C/S148V/S149V/ |
| | K154Q/W155Y/Y156R/E157P/D158R/W159A/ |
| | Y160F/K161D/S162D/M163W/V164Q/Q170H/ |
| | Y173R/Y177W/T181E/G193A/S194E/S195R/ |
| | Y221F/A222P/G227S/A230C/Q232M/P234S/ |
| | V235I/Q238N/R239K/A243V/V249T/M274C/ |
| | G277S/V279A/L280I/S281T/L282F/D284N |
| 346 | V249T/M274C/G277S/V279A/L280I/S281T/ |
| | L282F/D284N/V286A/E290Q/Q293P/H304E/ |
| | K309Y/H315Q/D329R/T335S/Q338E/Y351C/ |
| | A358G/G359V/Y360C/W361E/V362A/L364A/ |
| | K365Q/T368C/Y369F/I373M/A380G/S381M/ |
| | Q383S/M384L/R390E/A404S/G405A/V406L/ |
| | E407P/P408A/H409N/N412Q/L440C/G442R |
| 347 | P103N/L136C/S148V/S149V/K154Q/W155Y/ |
| | Y156R/E157P/D158R/W159A/Y160F/K161D/ |
| | S162D/M163W/V164Q/Q170H/Y173R/Y177W/ |
| | S194E/Y221F/P234S/R238K/A243V/V249T/ |
| | M274C/G277S/V279A/L280I/S281T/L282F/ |
| | D284N/H304E/K309Y/H315Q/D329R/T335S/ |
| | Q338E/Y351C/A358G/M384L/L440C/G442R |

Additionally, the enzyme having the amino acid sequence of SEQ ID NO: 348 and other variants of the polypeptide of SEQ ID NO: 349 are found to have a specific OTA detoxification activity of approximately 32 U/g and 19 U/g at pH 7.5 and at pH 6.0, respectively. Exemplary variants of the enzyme of SEQ ID NO: 349 comprising several amino acid substitutions are shown in Table 14 below, using the numbering of SEQ ID NO: 349 and comprising an N-terminal signal peptide analogous to the enzyme of SEQ ID NO: 349.

TABLE 14
Variants of the enzyme of SEQ ID NO: 349
comprising the indicated substitution(s)

| SEQ ID | |
|---|---|
| NO: | substitutions |
| 350 | S96V/G97E/A143S/A170P/F183I/ |
| | G307V/Y317F/M332L/V354L |
| 351 | A170P/F183I/L228I/Y317F/A328V/ |
| | A329V/M332I/G353A/E355P |
| 352 | A170P/F183I/L228I/G307V/Y317F/ |
| | A328V/A329L/G353A/E355F |
| 353 | E32R/N52P/S96V/G97V/N102D/ |
| | W103Y/Q118H/T129E/G141A/A143R/ |
| | Y169F/A170P/F183I/D186N/R187K/ |
| | M222C/S229T/E238Q/T283S/G307V/ |
| | A320I |
| 354 | W103Y/Q118H/T129E/G141A/A143R/ |
| | Y169F/A170P/F183I/D186N/R187K/ |
| | M222C/S229T/E238Q/T283S/G307V/ |
| | A320I/M332I/R338E/A352S/V354L/ |
| | E355P |
| 355 | E32R/N52P/S96V/G97V/N102D/W103Y/Q118H/ |
| | T129E/G141A/A143R/Y169F/A170P/F183I/D186N/ |
| | R187K/M222C/L228I/S229T/E238Q/T283S/G307V/ |
| | Y317F/A320I/A329V/M332I/R338E/A352S/V354L/ |
| | E355P |
| 356 | N52P/I63R/S96V/W103Y/Q118H/R124Y/T129E/ |
| | G141A/A143R/Y169F/A170P/F183I/D186N/R187K/ |
| | M222C/L228I/S229T/E238Q/D242E/T283S/G307V/ |
| | Y317F/A320I/A329V/M332I/R338E/A352S/V354L/ |
| | E355P |
| 357 | E32R/A38C/N52P/L84C/S96V/G97V/N102Q/ |
| | W103Y/Y104R/K105P/D106R/F107A/R108F/ |
| | D109K/R110D/M111W/L112Q/Q118H/Y121R/ |
| | N125W/T129E/G141A/S142E/A143R/Y169F/ |
| | A170P/G175S/G178C/G180M/P182S/F183I/ |
| | D186N/R187K/D191V/V197T/M222C/G225S/ |
| | V227A/L228I/S229T/L230F/D232N |
| 358 | V197T/M222C/G225S/V227A/L228I/S229T/L230F/ |
| | D232N/V234A/E238Q/Q241P/H252E/K257Y/ |
| | H263Q/D277R/T283S/T286E/Y299C/A306G/ |
| | G307V/E308C/W309E/V310A/M312A/K313Q/ |
| | N316C/Y317F/V321M/A328G/A329M/Q331S/ |
| | M332L/R338E/A352S/G353A/V354L/E355P/ |
| | P356A/H357N/N360Q/L388C/G390R |
| 359 | N52P/L84C/S96V/G97V/N102Q/W103Y/Y104R/ |
| | K105P/D106R/F107A/R108F/D109K/R110D/ |
| | M111W/L112Q/Q118H/Y121R/N125W/S142E/ |
| | Y169F/P182S/D186K/D191V/V197T/M222C/ |
| | G225S/V227A/L228I/S229T/L230F/D232N/ |
| | H252E/K257Y/H263Q/D277R/T283S/T286E/ |
| | Y299C/A306G/M332L/L388C/G390R |

### Example 4: Determination of Stability

Stability was determined in terms of thermostability. Notwithstanding, a person having skill in the art is aware that stability towards high temperature (e.g. more than 30° C.) often correlates with stability towards other non-standard conditions such as stability towards acidic or basic pH (e.g. below pH 6.5 or above pH 7.5, respectively), towards elevated salt concentrations, organic solvents or drying.

To determine the kinetic stability of the produced enzymes, thermostability assays were performed. To this end, purified protein samples were diluted to a protein concentration of 0.1 mg/mL in 20 mM Tris-HCl buffer pH 7.5 and aliquots were incubated in a thermocycler for 10 min at either 45, 55, 65, 75, 80, 85, 90, 95 or 99° C. After this incubation, the aliquots were stored at 4° C. until further analysis. A reference aliquot was stored at 4° C. throughout the incubation. Subsequently, all aliquots were assayed for residual OTA detoxification activity using the assay described in Example 3. Residual enzyme activities were calculated relative to the reference aliquot. The temperature range in which 50% of the initial activity was lost after the incubation (T50), in relation to the reference aliquot was determined. The results are shown in Table 15.

TABLE 15
Temperature at which 50% of the initial
activity was maintained (T50).

| SEQ ID NO: | T50 |
|---|---|
| 1 | 65-75 |
| 2 | >95 |
| 17 | 65-75 |
| 18 | 81-83 |
| 19 | 65 |
| 20 | <45 |
| 21 | 65-75 |
| 22 | >95 |
| 23 | >95 |
| 24 | >95 |
| 25 | >95 |
| 26 | >95 |
| 27 | >95 |
| 28 | >95 |
| 29 | >95 |
| 30 | >95 |
| 31 | >95 |
| 32 | >95 |
| 33 | >95 |
| 34 | >95 |
| 35 | 99 |
| 36 | 99 |
| 37 | >99 |
| 38 | >99 |
| 39 | >99 |
| 40 | >99 |
| 41 | 99 |
| 42 | >99 |
| 43 | >99 |
| 44 | >99 |
| 45 | >99 |
| 46 | >99 |
| 47 | >99 |
| 48 | <63.2 |
| 49 | 68-70 |
| 50 | 64 |
| 51 | 68-70 |
| 52 | <63.2 |
| 53 | <63.2 |
| 54 | <63.2 |
| 55 | 64-66 |
| 56 | 66-68 |
| 57 | 68-70 |
| 58 | 72 |
| 59 | 68-70 |
| 60 | 66-68 |
| 61 | 66-68 |
| 62 | 63-64 |
| 63 | 72-74 |
| 64 | 63-64 |
| 65 | 23102 |
| 66 | 65.3-66.9 |
| 67 | <63.2 |
| 68 | 70.7-72.6 |
| 69 | <63.2 |
| 70 | <63.2 |
| 71 | 70.7-72.6 |
| 72 | 74.4-77.0 |
| 73 | 74.4-77.0 |
| 74 | <63.2 |
| 75 | 74.4-77.0 |
| 77 | 65.3-66.9 |
| 78 | 68.8-70.7 |
| 79 | 70.7-72.6 |
| 80 | 64.0-65.3 |
| 368 | 68 |
| 369 | 68 |

Moreover, the enzyme having the amino acid sequence of SEQ ID NO: 336 and other variants of the polypeptide of SEQ ID NO: 337 are found to have a T50 of 90-95° C. Similarly, the enzyme having the amino acid sequence of SEQ ID NO: 348 and other variants of the polypeptide of SEQ ID NO: 349 are found to have a T50 of 85-90° C.

In comparison, an enzyme from Aspergillus niger having the amino acid sequence as laid out in the NCBI GenBank database record CAK41945.1 was found to have a T50 of 70-80° C.

To compare the temperature behavior and thermostability of selected enzymes to one another, enzyme was incubated at 65° C. for 10 min at pH 6.0. The enzymatic activity remaining after incubation was compared to the remaining activity of the enzyme of SEQ ID NO: 1 after incubation, the latter being set to 100%, see Table 16.

TABLE 16
Enzymatic activity comparison.

| SEQ ID NO: | % |
|---|---|
| 83 | 19 |
| 84 | 58 |
| 85 | 95 |
| 87 | 53 |
| 88 | 33 |
| 89 | 71 |
| 91 | 16 |
| 95 | 38 |
| 96 | 39 |
| 103 | 36 |
| 105 | 15 |
| 106 | 27 |
| 107 | 21 |
| 108 | 27 |
| 111 | 35 |
| 113 | 62 |
| 119 | 45 |
| 120 | 23 |
| 121 | 40 |
| 122 | 40 |
| 124 | 42 |
| 125 | 100 |
| 127 | 64 |
| 128 | 84 |
| 136 | 36 |
| 137 | 48 |
| 138 | 27 |
| 139 | 40 |
| 140 | 34 |
| 141 | 55 |
| 143 | 21 |
| 144 | 28 |
| 145 | 47 |
| 146 | 45 |
| 148 | 35 |
| 150 | 22 |
| 151 | 30 |
| 152 | 38 |
| 153 | 34 |
| 154 | 32 |
| 155 | 35 |
| 156 | 34 |
| 157 | 73 |
| 158 | 9 |
| 159 | 30 |
| 160 | 30 |
| 162 | 7 |
| 163 | 17 |
| 164 | 15 |
| 165 | 33 |
| 166 | 37 |
| 167 | 26 |
| 168 | 48 |
| 169 | 30 |
| 173 | 53 |
| 174 | 54 |
| 175 | 13 |
| 177 | 20 |
| 179 | 17 |
| 180 | 28 |
| 181 | 47 |
| 182 | 38 |
| 183 | 75 |
| 184 | 46 |
| 186 | 42 |
| 187 | 36 |
| 188 | 45 |
| 189 | 19 |
| 190 | 15 |
| 192 | 41 |
| 193 | 42 |
| 194 | 45 |
| 195 | 38 |
| 196 | 57 |
| 197 | 29 |
| 198 | 55 |
| 200 | 88 |
| 201 | 54 |
| 202 | 25 |
| 203 | 10 |
| 206 | 42 |
| 207 | 37 |
| 208 | 35 |
| 209 | 31 |
| 210 | 39 |
| 211 | 34 |
| 213 | 25 |
| 214 | 59 |
| 216 | 14 |
| 217 | 36 |
| 218 | 28 |
| 219 | 38 |
| 220 | 22 |
| 224 | 40 |
| 225 | 84 |
| 227 | 24 |
| 230 | 32 |
| 231 | 75 |
| 232 | 25 |
| 234 | 50 |
| 235 | 56 |
| 237 | 14 |
| 238 | 24 |
| 239 | 30 |
| 240 | 44 |
| 241 | 73 |
| 242 | 11 |
| 243 | 41 |
| 244 | 33 |
| 245 | 37 |
| 246 | 27 |
| 247 | 53 |
| 248 | 38 |
| 253 | 22 |
| 256 | 36 |
| 259 | 41 |
| 260 | 27 |
| 261 | 60 |
| 262 | 40 |
| 268 | 28 |
| 272 | 41 |
| 273 | 34 |
| 274 | 32 |
| 275 | 25 |
| 280 | 31 |
| 281 | 13 |
| 282 | 28 |
| 284 | 13 |
| 285 | 63 |
| 286 | 33 |
| 287 | 28 |
| 288 | 90 |
| 289 | 63 |
| 290 | 53 |
| 291 | 34 |
| 292 | 21 |
| 294 | 36 |
| 295 | 41 |
| 296 | 43 |
| 297 | 32 |
| 299 | 27 |
| 300 | 31 |
| 301 | 35 |
| 303 | 56 |
| 304 | 63 |
| 305 | 35 |
| 306 | 36 |
| 307 | 35 |
| 308 | 39 |
| 309 | 28 |
| 310 | 39 |
| 311 | 30 |
| 312 | 17 |
| 313 | 20 |
| 315 | 29 |
| 316 | 25 |

### Example 5: Pelleting Trial

To verify the suitability of the enzymes referred to herein as additives e.g. for feed or food, pelleting trials were conducted. As an example, 1.15 U or 0.06 U of either of the enzymes having either the amino acid sequence of SEQ ID NO: 1 or 2 was homogeneously mixed with one kg of mash feed and subjected to standard feed pelleting process where the temperature of the conditioner was set to either 75, 80, 85, 90 or 95° C. in five separate batches of feed pelleting. The effect of pelleting process on the stability of enzyme was estimated by calculation of percentage degradation of spiked OTA to OTalpha in resuspended pellets. Untreated mash was analyzed applying the following protocol: 50 μL of 10 ppm OTA were spiked to g of dry sample, directly before starting the assay by adding 4.95 mL RO-water (37° C.). The suspension was incubated in 50 mL tubes in an overhead shaker at 37° C. for 60 min. The reaction was stopped by adding 1 mL 6 M phosphoric acid followed by another incubation in a rotary shaker at room temperature for 10 min. Subsequently 30 μL of ¹³C-labeled OTA/¹³C-labeled OTalpha (each 1 ppm) internal standard were added to each sample and mixed. 30 mL ethyl acetate were added followed by shaking for 60 min at 80 rpm on an overhead shaker. After centrifugation (10 min, 3214 rcf), 8 mL of the organic upper phase were transferred to a new 50 mL tube. 4 mL of 0.1 M NaHCO3 (pH 8.2) were added. After shaking for 1 min at 80 rpm on an overhead shaker, sample was centrifuged again (10 min, 3214 rcf) and 3 mL of the aqueous lower phase transferred to a new 15 mL tube. 70 μL 85% phosphoric acid and 1.5 mL ethyl acetate were added and shaken for 1 min at 80 rpm at an overhead shaker. After centrifugation (10 min, 3214 rcf) 50 μL organic upper phase were added to 50 μL acetonitrile in a silanized HPLC vial and thoroughly mixed. OTA and OTalpha content was determined by HPLC MS/MS method as described by Dellafiora et al. (2020, Toxins 12, 258) to determine the percentage of OTA degradation to form OTalpha. Table 17 exemplarily shows the amounts of remaining OTA and formed OTalpha in an untreated mash feed sample (blank), and after pelleting at either of the indicated temperatures when the feed sample is treated with either of the indicated enzymes. Concluding, heat treatment during pelleting does not affect the enzymes' ability for degrading OTA into OTalpha.

TABLE 17
OTA degradation ability before pelleting and after pelleting at the indicated temperature

| Samples | OTA % | OTalpha % | Samples | OTA % | OTalpha % |
|---|---|---|---|---|---|
| Mash blank | 100 | 0 | Mash blank | 100 | 0 |
| Mash + SEQ ID NO: | 20 | 80 | Mash + SEQ ID NO: | 1 | 99 |
| 2_before pelleting | | | 1_before pelleting | | |
| Mash + SEQ ID NO: | 16 | 84 | Mash + SEQ ID NO: | 1 | 99 |
| 2_pellet_75° C. | | | 1_pellet_75° C. | | |
| Mash + SEQ ID NO: | 12 | 88 | Mash + SEQ ID NO: | 1 | 99 |
| 2_pellet_80° C. | | | 1_pellet_80° C. | | |
| Mash + SEQ ID NO: | 16 | 84 | Mash + SEQ ID NO: | 1 | 99 |
| 2_pellet_85° C. | | | 1_pellet_85° C. | | |
| Mash + SEQ ID NO: | 12 | 88 | Mash + SEQ ID NO: | 1 | 99 |
| 2_pellet_90° C. | | | 1_pellet_90° C. | | |
| Mash + SEQ ID NO: | 13 | 87 | Mash + SEQ ID NO: | 1 | 99 |
| 2_pellet_95° C. | | | 1_pellet_95° C. | | |

### Example 6: Feeding Trial

To further study an application of the enzymes referred to herein as additives e.g. for feed or food, feeding trials were conducted in pigs. Exemplarily, either of the enzymes having either the amino acid sequence of SEQ ID NO: 1 or 2 were produced by cultivation of E. coli transformants in bioreactors by applying methods known to a person having skill in the art. The cell broth was homogenized and centrifuged. The supernatant was concentrated, dried by lyophilization and formulated as a powder by grinding. The enzymatic activity of the thus obtained lyophilized powder was determined, and the powder was mixed with feed for the feeding trial as described in the following.

Thirteen piglet groups were studied based on the following experimental diet setups: 1) Base feed; 2) base feed+50 ppb OTA; 3) base feed+50 ppb OTA+2.62 U of the enzyme of SEQ ID NO: 1 per kg of feed; 4) base feed+50 ppb OTA+0.29 U of the enzyme of SEQ ID NO: 2 per kg of feed; 5) base feed+500 ppb OTA; 6) base feed+500 ppb OTA+2.62 U of the enzyme of SEQ ID NO: 1 per kg of feed; 7) 500 ppb OTA+0.29 U of the enzyme of SEQ ID NO: 2 per kg of feed. The base feed itself did not contain any detectable amount of OTA.

Each trial group consisted of four piglets weighing approximately 7-8 kg, who were coming out from weaning and adapted to solid diet before being fed the experimental diet as described above. The duration of the feeding trial (i.e. actual days of feeding the experimental diet) was fourteen days. Collectively each trial group consumed around 15 kg of feed. No piglets fell sick to any unexpected disease throughout the feeding trial. Urine and blood samples were collected on day 0, just before starting the experimental diet, and on day 14.

Extraction of OTA was performed from plasma and urine samples for subsequent analysis by HPLC-MS. 200 μL of sample was thoroughly mixed with 800 μL of extraction solution (containing 99% of ethyl acetate and 1% of phosphoric acid, 85%) followed by centrifugation at 19000×g for 5 min for phase separation. 50 μL of upper phase was diluted with 50 μL of acetonitrile, followed by HPLC-MS analysis. The HPLC-MS method as described by Dellafiora et al. (2020, Toxins 12, 258) was used to determine the changes in OTA concentration in the samples taken during the trial upon consumption of the experimental diets. The results are shown in Table 18 using the diet setup numbering as indicated above. Notably, a reduction of the OTA concentration in both, plasma and urine, was observed only when recombinant enzyme was part of the diet setup, demonstrating the applicability of the enzymes of the present invention in food/feed for OTA detoxification.

TABLE 18
OTA concentrations (in ppb) in plasma and blood samples on
day 0 and day 14, and OTA reduction (in %).

| | Plasma samples | | | Urine samples | | |
|---|---|---|---|---|---|---|
| | | | % OTA | | | % OTA |
| Diet setup # | Day 0 | Day 14 | reduction | Day 0 | Day 14 | reduction |
| 1 | 0 | 0 | n/a | 0 | 0 | n/a |
| 2 | 0 | 41 ± 4 | n/a | 0 | 2 | n/a |
| 3 | 0 | 19 ± 2 | 54 | 0 | 0 | 100 |
| 4 | 0 | 11 ± 3 | 73 | 0 | 0 | 100 |
| 5 | 0 | 414 ± 72 | n/a | 0 | 20 ± 2 | n/a |
| 6 | 0 | 168 ± 41 | 59 | 0 | 7 ± 1 | 65 |
| 7 | 0 | 107 ± 31 | 74 | 0 | 4 | 80 |

1. A method (e.g., in vitro, ex vivo, in vivo or manufacturing method) for detoxifying (e.g., modifying or hydrolyzing) a mycotoxin having Formula I (e.g., Ochratoxin A, Ochratoxin B and/or Ochratoxin C): 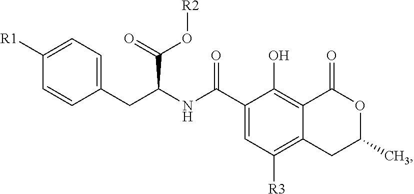
wherein R1 is selected from the group consisting of H and OH, R2 is selected from the group consisting of H and CH₂—CH₃ and R3 is selected from the group consisting of H and Cl;
said method comprising:
(a) providing:
(i) one or more polypeptides having at least 70% sequence identity (e.g. at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) to the amino acid sequence selected from the group consisting of: SEQ ID NO: 1, SEQ ID NO: 2, SEQ ID NO: 22, SEQ ID NO: 318, SEQ ID NO: 336, SEQ ID NO: 348, SEQ ID NOs: 16-359, 368-370; wherein said one or more polypeptide are capable of detoxifying (e.g., modifying or hydrolyzing) at least one mycotoxin having said Formula I; preferably said polypeptide/s having peptidase activity having EC 3.4.13.X, wherein X is selected from the group consisting of: 9, 4, 5, 7, 12, 17, 18, 19, 20, 21, 22, 23; further preferably said polypeptide/s having a peptidase activity having EC 3.4.13.9; most preferably said one or more polypeptide comprising one or more of the following amino acid sequences (e.g., conservative motifs):
i′) PGFIDAHVH (SEQ ID NO: 3), preferably said SEQ ID NO: 3 is comprised at amino acid positions corresponding to positions 87-95 of SEQ ID NO: 1 (e.g., using the numbering of SEQ ID NO: 1);
ii′) PAEQA (SEQ ID NO: 4), preferably said SEQ ID NO: 4 is comprised at amino acid positions corresponding to positions 116-120 of SEQ ID NO: 1 (e.g., using the numbering of SEQ ID NO: 1);
iii′) GVCNG (SEQ ID NO: 5), preferably said SEQ ID NO: 5 is comprised at amino acid positions corresponding to positions 197-201 of SEQ ID NO: 1 (e.g., using the numbering of SEQ ID NO: 1);
iv′) CRAAVR (SEQ ID NO: 6), preferably said SEQ ID NO: 6 is comprised at amino acid positions corresponding to positions 205-210 of SEQ ID NO: 1 (e.g., using the numbering of SEQ ID NO: 1);
v′) IKFMPSGGVLSL (SEQ ID NO: 7), preferably said SEQ ID NO: 7 is comprised at amino acid positions corresponding to positions 220-231 of SEQ ID NO: 1 (e.g., using the numbering of SEQ ID NO: 1);
vi′) RKVAAH (SEQ ID NO: 8), preferably said SEQ ID NO: 8 is comprised at amino acid positions corresponding to positions 257-262 of SEQ ID NO: 1 (e.g., using the numbering of SEQ ID NO: 1);
vii′) AGVDSIEHG (SEQ ID NO: 9), preferably said SEQ ID NO: 9 is comprised at amino acid positions corresponding to positions 275-283 of SEQ ID NO: 1 (e.g., using the numbering of SEQ ID NO: 1);
viii′) VMPGLIDAH (SEQ ID NO: 10), preferably said SEQ ID NO: 10 is comprised at amino acid positions corresponding to positions 51-59 of SEQ ID NO: 2 (e.g., using the numbering of SEQ ID NO: 2);
ix′) AGFTTVRDCG (SEQ ID NO: 11), preferably said SEQ ID NO: 11 is comprised at amino acid positions corresponding to positions 96-105 of SEQ ID NO: 2 (e.g., using the numbering of SEQ ID NO: 2);
x′) QTFGH (SEQ ID NO: 12), preferably said SEQ ID NO: 12 is comprised at amino acid positions corresponding to positions 135-139 of SEQ ID NO: 2 (e.g., using the numbering of SEQ ID NO: 2);
xi′) ATGGVLS (SEQ ID NO: 13), preferably said SEQ ID NO: 13 is comprised at amino acid positions corresponding to positions 185-191 of SEQ ID NO: 2 (e.g., using the numbering of SEQ ID NO: 2);
xii′) YVAAHAHGD (SEQ ID NO: 14), preferably said SEQ ID NO: 14 is comprised at amino acid positions corresponding to positions 219-227 of SEQ ID NO: 2 or 22 (e.g., using the numbering of SEQ ID NO: 2 or 22);
xiii′) LTPTL (SEQ ID NO: 15), preferably said SEQ ID NO: 15 is comprised at amino acid positions corresponding to positions 262-266 of SEQ ID NO: 2 or 22 (e.g., using the numbering of SEQ ID NO: 2 or 22);
xiv′) VMPGLID (SEQ ID NO: 360), preferably said SEQ ID NO: 360 is comprised at amino acid positions corresponding to positions 52-58 of SEQ ID NO: 22 (e.g., using the numbering of SEQ ID NO: 22);
xv′) KGGSL (SEQ ID NO: 361), preferably said SEQ ID NO: 361 is comprised at amino acid positions corresponding to positions 67-71 of SEQ ID NO: 22 (e.g., using the numbering of SEQ ID NO: 22);
xvi′) LLLAGFTTVRDCG (SEQ ID NO: 362), preferably said SEQ ID NO: 362 is comprised at amino acid positions corresponding to positions 93-105 of SEQ ID NO: 22 (e.g., using the numbering of SEQ ID NO: 22);
xvii′) QTFGHGE (SEQ ID NO: 363), preferably said SEQ ID NO: 363 is comprised at amino acid positions corresponding to positions 135-141 of SEQ ID NO: 22 (e.g., using the numbering of SEQ ID NO: 22);
xviii′) FATGGVLSQRD (SEQ ID NO: 364), preferably said SEQ ID NO: 364 is comprised at amino acid positions corresponding to positions 184-194 of SEQ ID NO: 22 (e.g., using the numbering of SEQ ID NO: 22);
xix′) IVNEA (SEQ ID NO: 365), preferably said SEQ ID NO: 365 is comprised at amino acid positions corresponding to positions 209-213 of SEQ ID NO: 22 (e.g., using the numbering of SEQ ID NO: 22);
xx′) IGVDEWGL (SEQ ID NO: 366), preferably said SEQ ID NO: 366 is comprised at amino acid positions corresponding to positions 279-286 of SEQ ID NO: 22 (e.g., using the numbering of SEQ ID NO: 22);
xxi′) GFETGL (SEQ ID NO: 367), preferably said SEQ ID NO: 367 is comprised at amino acid positions corresponding to positions 317-322 of SEQ ID NO: 22 (e.g., using the numbering of SEQ ID NO: 22);
ii) one or more variants of a parent polypeptide, wherein said variant/s comprising an alteration (e.g., a substitution, deletion and/or insertion) at one or more positions corresponding to positions of the parent polypeptide, wherein said variant/s having at least 70% (e.g. at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%), but less than 100% sequence identity to a parent amino acid sequence selected from the group consisting of: SEQ ID NOs 1, 2, 16-21, 22, 23-34, 317-318, 326-335, 336-337, 348-349, 368-370; wherein said variant/s are capable of detoxifying (e.g., modifying or hydrolyzing) at least one mycotoxin having said Formula I (e.g., Ochratoxin A, Ochratoxin B and/or Ochratoxin C); preferably said variant/s having peptidase activity having EC 3.4.13.X, wherein X is selected from the group consisting of: 9, 4, 5, 7, 12, 17, 18, 19, 20, 21, 22, 23; further preferably said variant/s having a peptidase activity having EC 3.4.13.9; most preferably said variant/s comprising one or more of the following amino acid sequences (e.g., conservative motifs): (i′)-(xxi′) according to (i); further most preferably said parent polypeptide is selected from the group consisting of: SEQ ID NOs 1, 2, 16-21, 22, 23-34, 317-318, 326-335, 336-337, 348-349, 368-370;
(iii) one or more polynucleotides, nucleic acid constructs and/or expression vectors encoding and/or capable of expressing one or more polypeptides and/or one or more variants according to (i)-(ii);
(iv) one or more recombinant host cells, spores, transgenic plants, transgenic seeds and/or transgenic pollen grains comprising (i), (ii) and/or (iii); and/or
(v) foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent, intermediate detoxifying agent; nutritional supplement, intermediate nutritional supplement; prebiotic, intermediate prebiotic and/or mixture/s thereof comprising (i), (ii), (iii) and/or (iv);
(b) applying (a) to said mycotoxin having Formula I.

2. A foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent, intermediate detoxifying agent; nutritional supplement, intermediate nutritional supplement; prebiotic, intermediate prebiotic and/or mixture/s thereof, comprising one or more of the following:
(i) one or more polypeptides having at least 70% sequence identity (e.g. at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) to the amino acid sequence selected from the group consisting of: SEQ ID NO: 1, SEQ ID NO: 2, SEQ ID NO: 22, SEQ ID NO: 318, SEQ ID NO: 336, SEQ ID NO: 348, SEQ ID NOs: 16-359, 368-370; wherein said polypeptide/s are capable of detoxifying (e.g., modifying or hydrolyzing) at least one mycotoxin having said Formula I (e.g., Ochratoxin A, Ochratoxin B and/or Ochratoxin C): 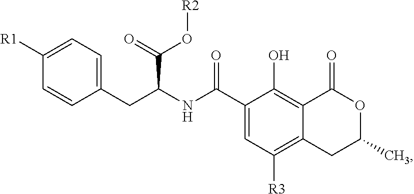
wherein R1 is selected from the group consisting of H and OH, R2 is selected from the group consisting of H and CH₂—CH₃ and R3 is selected from the group consisting of H and Cl; preferably said polypeptide/s having a peptidase activity having EC 3.4.13.X, wherein X is selected from the group consisting of: 9, 4, 5, 7, 12, 17, 18, 19, 20, 21, 22, 23; further preferably said polypeptide/s having a peptidase activity having EC 3.4.13.9; most preferably said polypeptide/s comprising one or more of the following amino acid sequences (e.g., conservative motifs):
i′) PGFIDAHVH (SEQ ID NO: 3), preferably said SEQ ID NO: 3 is comprised at amino acid positions corresponding to positions 87-95 of SEQ ID NO: 1 (e.g., using the numbering of SEQ ID NO: 1);
ii′) PAEQA (SEQ ID NO: 4), preferably said SEQ ID NO: 4 is comprised at amino acid positions corresponding to positions 116-120 of SEQ ID NO: 1 (e.g., using the numbering of SEQ ID NO: 1);
iii′) GVCNG (SEQ ID NO: 5), preferably said SEQ ID NO: 5 is comprised at amino acid positions corresponding to positions 197-201 of SEQ ID NO: 1 (e.g., using the numbering of SEQ ID NO: 1);
iv′) CRAAVR (SEQ ID NO: 6), preferably said SEQ ID NO: 6 is comprised at amino acid positions corresponding to positions 205-210 of SEQ ID NO: 1 (e.g., using the numbering of SEQ ID NO: 1);
v′) IKFMPSGGVLSL (SEQ ID NO: 7), preferably said SEQ ID NO: 7 is comprised at amino acid positions corresponding to positions 220-231 of SEQ ID NO: 1 (e.g., using the numbering of SEQ ID NO: 1);
vi′) RKVAAH (SEQ ID NO: 8), preferably said SEQ ID NO: 8 is comprised at amino acid positions corresponding to positions 257-262 of SEQ ID NO: 1 (e.g., using the numbering of SEQ ID NO: 1);
vii′) AGVDSIEHG (SEQ ID NO: 9), preferably said SEQ ID NO: 9 is comprised at amino acid positions corresponding to positions 275-283 of SEQ ID NO: 1 (e.g., using the numbering of SEQ ID NO: 1);
viii′) VMPGLIDAH (SEQ ID NO: 10), preferably said SEQ ID NO: 10 is comprised at amino acid positions corresponding to positions 51-59 of SEQ ID NO: 2 (e.g., using the numbering of SEQ ID NO: 2);
ix′) AGFTTVRDCG (SEQ ID NO: 11), preferably said SEQ ID NO: 11 is comprised at amino acid positions corresponding to positions 96-105 of SEQ ID NO: 2 (e.g., using the numbering of SEQ ID NO: 2);
x′) QTFGH (SEQ ID NO: 12), preferably said SEQ ID NO: 12 is comprised at amino acid positions corresponding to positions 135-139 of SEQ ID NO: 2 (e.g., using the numbering of SEQ ID NO: 2);
xi′) ATGGVLS (SEQ ID NO: 13), preferably said SEQ ID NO: 13 is comprised at amino acid positions corresponding to positions 185-191 of SEQ ID NO: 2 (e.g., using the numbering of SEQ ID NO: 2);
xii′) YVAAHAHGD (SEQ ID NO: 14), preferably said SEQ ID NO: 14 is comprised at amino acid positions corresponding to positions 219-227 of SEQ ID NO: 2 or 22 (e.g., using the numbering of SEQ ID NO: 2 or 22);
xiii′) LTPTL (SEQ ID NO: 15), preferably said SEQ ID NO: 15 is comprised at amino acid positions corresponding to positions 262-266 of SEQ ID NO: 2 or 22 (e.g., using the numbering of SEQ ID NO: 2 or 22);
xiv′) VMPGLID (SEQ ID NO: 360), preferably said SEQ ID NO: 360 is comprised at amino acid positions corresponding to positions 52-58 of SEQ ID NO: 22 (e.g., using the numbering of SEQ ID NO: 22);
xv′) KGGSL (SEQ ID NO: 361), preferably said SEQ ID NO: 361 is comprised at amino acid positions corresponding to positions 67-71 of SEQ ID NO: 22 (e.g., using the numbering of SEQ ID NO: 22);
xvi′) LLLAGFTTVRDCG (SEQ ID NO: 362), preferably said SEQ ID NO: 362 is comprised at amino acid positions corresponding to positions 93-105 of SEQ ID NO: 22 (e.g., using the numbering of SEQ ID NO: 22);
xvii′) QTFGHGE (SEQ ID NO: 363), preferably said SEQ ID NO: 363 is comprised at amino acid positions corresponding to positions 135-141 of SEQ ID NO: 22 (e.g., using the numbering of SEQ ID NO: 22);
xviii′) FATGGVLSQRD (SEQ ID NO: 364), preferably said SEQ ID NO: 364 is comprised at amino acid positions corresponding to positions 184-194 of SEQ ID NO: 22 (e.g., using the numbering of SEQ ID NO: 22);
xix′) IVNEA (SEQ ID NO: 365), preferably said SEQ ID NO: 365 is comprised at amino acid positions corresponding to positions 209-213 of SEQ ID NO: 22 (e.g., using the numbering of SEQ ID NO: 22);
xx′) IGVDEWGL (SEQ ID NO: 366), preferably said SEQ ID NO: 366 is comprised at amino acid positions corresponding to positions 279-286 of SEQ ID NO: 22 (e.g., using the numbering of SEQ ID NO: 22);
xxi′) GFETGL (SEQ ID NO: 367), preferably said SEQ ID NO: 367 is comprised at amino acid positions corresponding to positions 317-322 of SEQ ID NO: 22 (e.g., using the numbering of SEQ ID NO: 22).
ii) one or more variants of a parent polypeptide, wherein said variant/s comprising an alteration (e.g., a substitution, deletion and/or insertion) at one or more positions corresponding to positions of the parent polypeptide, wherein said variant/s having at least 70% (e.g. at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%), but less than 100% sequence identity to a parent amino acid sequence selected from the group consisting of: SEQ ID NOs 1, 2, 16-21, 22, 23-34, 317-318, 326-335, 336-337, 348-349, 368-370; wherein said variant/s are capable of detoxifying (e.g., modifying or hydrolyzing) at least one mycotoxin having said Formula I (e.g., Ochratoxin A, Ochratoxin B and/or Ochratoxin C); preferably said variant/s having a peptidase activity having EC 3.4.13.X, wherein X is selected from the group consisting of: 9, 4, 5, 7, 12, 17, 18, 19, 20, 21, 22, 23; further preferably said variant/s having a peptidase activity having EC 3.4.13.9; most preferably said variant/s comprising one or more of the following amino acid sequences (e.g., conservative motifs): (i′)-(xxi′) according to (i);
(iii) one or more polynucleotides, nucleic acid constructs and/or expression vectors encoding and/or capable of expressing one or more polypeptides and/or one or more variants according to (i)-(ii); and/or
(iv) one or more recombinant host cells, spores, transgenic plants, transgenic seeds and/or transgenic pollen grains comprising (i), (ii) and/or (iii).

3. A variant of a parent polypeptide capable of detoxifying (e.g., modifying or hydrolyzing) at least one mycotoxin having said Formula I (e.g., Ochratoxin A, Ochratoxin B and/or Ochratoxin C): 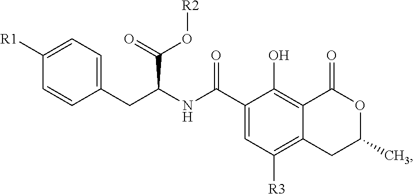
wherein R1 is selected from the group consisting of H and OH, R2 is selected from the group consisting of H and CH₂—CH₃ and R3 is selected from the group consisting of H and Cl; wherein said variant comprising an alteration (e.g., a substitution, deletion and/or insertion) at one or more positions corresponding to positions of the parent polypeptide, wherein said variant/s having at least 70% (e.g. at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%), but less than 100% sequence identity to a parent amino acid sequence selected from the group consisting of: SEQ ID NOs 1, 2, 16-21, 22, 23-34, 317-318, 326-335, 336-337, 348-349, 368-370; preferably said variant having a peptidase activity having EC 3.4.13.X, wherein X is selected from the group consisting of: 9, 4, 5, 7, 12, 17, 18, 19, 20, 21, 22, 23; further preferably said variant having a peptidase activity having EC 3.4.13.9; most preferably, said variant comprising one or more of the following amino acid sequences (e.g., conservative motifs):
i′) PGFIDAHVH (SEQ ID NO: 3), preferably said SEQ ID NO: 3 is comprised at amino acid positions corresponding to positions 87-95 of SEQ ID NO: 1 (e.g., using the numbering of SEQ ID NO: 1);
ii′) PAEQA (SEQ ID NO: 4), preferably said SEQ ID NO: 4 is comprised at amino acid positions corresponding to positions 116-120 of SEQ ID NO: 1 (e.g., using the numbering of SEQ ID NO: 1);
iii′) GVCNG (SEQ ID NO: 5), preferably said SEQ ID NO: 5 is comprised at amino acid positions corresponding to positions 197-201 of SEQ ID NO: 1 (e.g., using the numbering of SEQ ID NO: 1);
iv′) CRAAVR (SEQ ID NO: 6), preferably said SEQ ID NO: 6 is comprised at amino acid positions corresponding to positions 205-210 of SEQ ID NO: 1 (e.g., using the numbering of SEQ ID NO: 1);
v′) IKFMPSGGVLSL (SEQ ID NO: 7), preferably said SEQ ID NO: 7 is comprised at amino acid positions corresponding to positions 220-231 of SEQ ID NO: 1 (e.g., using the numbering of SEQ ID NO: 1);
vi′) RKVAAH (SEQ ID NO: 8), preferably said SEQ ID NO: 8 is comprised at amino acid positions corresponding to positions 257-262 of SEQ ID NO: 1 (e.g., using the numbering of SEQ ID NO: 1);
vii′) AGVDSIEHG (SEQ ID NO: 9), preferably said SEQ ID NO: 9 is comprised at amino acid positions corresponding to positions 275-283 of SEQ ID NO: 1 (e.g., using the numbering of SEQ ID NO: 1);
viii′) VMPGLIDAH (SEQ ID NO: 10), preferably said SEQ ID NO: 10 is comprised at amino acid positions corresponding to positions 51-59 of SEQ ID NO: 2 (e.g., using the numbering of SEQ ID NO: 2);
ix′) AGFTTVRDCG (SEQ ID NO: 11), preferably said SEQ ID NO: 11 is comprised at amino acid positions corresponding to positions 96-105 of SEQ ID NO: 2 (e.g., using the numbering of SEQ ID NO: 2);
x′) QTFGH (SEQ ID NO: 12), preferably said SEQ ID NO: 12 is comprised at amino acid positions corresponding to positions 135-139 of SEQ ID NO: 2 (e.g., using the numbering of SEQ ID NO: 2);
xi′) ATGGVLS (SEQ ID NO: 13), preferably said SEQ ID NO: 13 is comprised at amino acid positions corresponding to positions 185-191 of SEQ ID NO: 2 (e.g., using the numbering of SEQ ID NO: 2);
xii′) YVAAHAHGD (SEQ ID NO: 14), preferably said SEQ ID NO: 14 is comprised at amino acid positions corresponding to positions 219-227 of SEQ ID NO: 2 (e.g., using the numbering of SEQ ID NO: 2);
xiii′) LTPTL (SEQ ID NO: 15), preferably said SEQ ID NO: 15 is comprised at amino acid positions corresponding to positions 262-266 of SEQ ID NO: 2 (e.g., using the numbering of SEQ ID NO: 2);
xiv′) VMPGLID (SEQ ID NO: 360), preferably said SEQ ID NO: 360 is comprised at amino acid positions corresponding to positions 52-58 of SEQ ID NO: 22 (e.g., using the numbering of SEQ ID NO: 22);
xv′) KGGSL (SEQ ID NO: 361), preferably said SEQ ID NO: 361 is comprised at amino acid positions corresponding to positions 67-71 of SEQ ID NO: 22 (e.g., using the numbering of SEQ ID NO: 22);
xvi′) LLLAGFTTVRDCG (SEQ ID NO: 362), preferably said SEQ ID NO: 362 is comprised at amino acid positions corresponding to positions 93-105 of SEQ ID NO: 22 (e.g., using the numbering of SEQ ID NO: 22);
xvii′) QTFGHGE (SEQ ID NO: 363), preferably said SEQ ID NO: 363 is comprised at amino acid positions corresponding to positions 135-141 of SEQ ID NO: 22 (e.g., using the numbering of SEQ ID NO: 22);
xviii′) FATGGVLSQRD (SEQ ID NO: 364), preferably said SEQ ID NO: 364 is comprised at amino acid positions corresponding to positions 184-194 of SEQ ID NO: 22 (e.g., using the numbering of SEQ ID NO: 22);
xix′) IVNEA (SEQ ID NO: 365), preferably said SEQ ID NO: 365 is comprised at amino acid positions corresponding to positions 209-213 of SEQ ID NO: 22 (e.g., using the numbering of SEQ ID NO: 22);
xx′) IGVDEWGL (SEQ ID NO: 366), preferably said SEQ ID NO: 366 is comprised at amino acid positions corresponding to positions 279-286 of SEQ ID NO: 22 (e.g., using the numbering of SEQ ID NO: 22);
xxi′) GFETGL (SEQ ID NO: 367), preferably said SEQ ID NO: 367 is comprised at amino acid positions corresponding to positions 317-322 of SEQ ID NO: 22 (e.g., using the numbering of SEQ ID NO: 22).

4. The method, variant, foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive), detoxifying agent, intermediate detoxifying agent, nutritional supplement, intermediate nutritional supplement; prebiotic, intermediate prebiotic and/or mixture/s thereof according to any one of the preceding claims, wherein said polypeptide and/or variant having one or more of the following:
i) said peptidase activity is a Xaa-Pro dipeptidase EC 3.4.13.9;
ii) said polypeptide and/or variant having a TIM barrel structure comprising 8 α-helices and 8 parallel β-strands alternating along the polypeptide backbone, wherein preferably said TIM barrel further comprising a phosphate binding site;
iii) said polypeptide and/or variant having a specific activity of at least 2.9 U/g at pH7.5;
iv) said polypeptide and/or variant having a specific activity of at least 0.9 U/g at pH6.0;
v) said polypeptide and/or variant having a T50 value of more than 80° C. (e.g. more than 85° C.);
vi) said polypeptide is selected from the group consisting of: SEQ ID NOs 1, 2, 16-21, 22, 23-34, 317-318, 326-335, 336-337, 348-349, 368-370; and/or
vii) said variant is selected from the group consisting of: SEQ ID NOs: 35-316, 319-325, 338-347, 350-359.

5. The method, variant, foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive);
detoxifying agent, intermediate detoxifying agent; nutritional supplement, intermediate nutritional supplement;
prebiotic, intermediate prebiotic and/or mixture/s thereof according to any one of the preceding claims, wherein said variant comprising an alteration (e.g., a substitution, deletion and/or insertion) at one or more positions corresponding to positions 65,106,132, 268, 269, 272, 275 and/or 318 of SEQ ID NO: 2, preferably using the numbering of SEQ ID NO: 2.

6. The method, variant, foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent, intermediate detoxifying agent; nutritional supplement, intermediate nutritional supplement; prebiotic, intermediate prebiotic and/or mixture/s thereof according to claim 5, wherein said variant comprising one or more of the following substitutions or combinations of substitutions: I65G, I65A, E106G, E106S, P132A, I268A, Q269I, I272V, Y275V; F318V; I272V; Y275F; Y275H; and/or F318V; or equivalent amino acid substitutions thereof.

7. The method, variant, foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent, intermediate detoxifying agent; nutritional supplement, intermediate nutritional supplement; prebiotic, intermediate prebiotic and/or mixture/s thereof according to any one of the preceding claims, wherein said variant comprising an alteration (e.g., a substitution, deletion and/or insertion) at one or more positions corresponding to positions 30, 36, 50, 85, 97, 98, 99, 100, 103, 104, 105, 106, 107, 108, 109, 111, 112, 113, 119, 122, 126, 130, 142, 143, 144, 170, 171, 176, 178, 179, 181, 183, 184, 187, 188, 192, 198, 223, 226, 228, 229, 230, 231, 233, 235, 239, 242, 253, 264, 284, 287, 307, 308, 310, 311, 314, 315, 317, 318, 322, 329, 330, 332, 333, 339, 353, 354, 355, 356, 357, 358 or 361 of SEQ ID NOs: 1 or 318, preferably using the numbering of SEQ ID NO: 1 or 318, further preferably using the numbering of SEQ ID NO: 318.

8. The method, variant, foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent, intermediate detoxifying agent; nutritional supplement, intermediate nutritional supplement; prebiotic, intermediate prebiotic and/or mixture/s thereof according to claim 7, wherein said variant comprising one or more of the following substitutions or combinations of substitutions: Y300C+L389C; I308V; A171P; T144A; V355L; I308A; G354A; S143E; M223C; A171G; H187N; I184V; H187Q; E239Q; V311I; K314Q; F109A; Y122R; F318Y; A36C+L85C; A375P; M223C; M223A; H264Q; A307G; H178Q; A307G+V355T; T144A; A171P; A171G; M223C; V228A; L229V; L229M; A307S; A307G; I308V; V311T; V355T; V311A; V355A; H264Q; M223C; M223A; A171L; A171G; H178Q; H178L; A307G+V311A; A307G+V311T; A307G+V355T; A307G+A355A; H178S; V228L; V2281; L229I; H264N; H264L; A307V; A307T; I308A; V311L; V311I; G354A; S97D; S97N; S97P; S97T; S97V; G142A; G142P; T144D; T144E; T144P; T144R; A179P; A179S; A179T; A171P+I184V; A171P+I308A; A171P+I308V; A171P+F318Y; A171P+G354A; I184V+I308A; I184V+I308V; I184V+F318Y; I184V+G354A; I308A+F318Y; I308V+F318Y; I308A+G354A; I308V+G354A; F318Y+G354A; A171P+I184V+I308A; A171P+I184V+I308V; A171P+I184V+F318Y; A171P+I184V+G354A; A171P+I308A+F318Y; A171P+I308V+F318Y; A171P+I308A+G354A; A171P+I308V+G354A; A171P+F318Y+G354A; I184V+I308A+F318Y; I184V+I308V+F318Y; I184V+I308A+G354A; I184V+I308V+G354A; I184V+F318Y+G354A; I308A+F318Y+G354A; I308V+F318Y+G354A; Q181M; Q181L; F318L; F318M; A329G; A329V; V355L; V355I; Y170E; Y170F; Y170R; A330L; A330M; A330V; L333I; L333M; L333V; G354L; G354S; G354T; G354V; G356A; G356F; G356P; G356V; G356Y; F109A; P183G; P183S; F108V; F108L; F109V; F109L; M112I; M112W; I184V; I184F; W104F; W104L; W104P; W104Y; I322L; I322M; I322Q; I322S; H187Q; H187N; H310Q; H310N; K314Q; K314E; H332Q; H332V; E287Q; H358Q; H358Y; Q99D; H30E; H30K; H30P; H30R; H50D; H50N; H50P; S100D; N103D; N103L; N103Q; T130E; H187D; H187E; H187G; H187P; K188D; K188E; K188R; G226A; G226P; G226S; S230D; S230P; S230T; D233N; D233Q; D233S; H253A; H253N; S284D; S284H; S284T; H310D; H310E; H332E; H332R; H332S; H339E; H339K; H339N; H339S; A353S; H358A; H358K; H358N; N361E; N361Q; D110K+K188D; D110K+K188E; H187N+K188R; S284D+A329S; S284D+A329T; H310D+E313A; H310E+E313A; V235P; E239Q; A357P; F108Y; N111D; M113Q; Y122R; Y126W; F109Y; Q242P; I315P; T317Y; F318Y; G98S; G98V; Y105N; Y105R; G106P; D107R; D107Y; M112F; M112K; M113D; M113R; Q119E; Q119H; Q119K; Q119M; G176S; A192L; A192M; A192V; V198T; L231F; V235A; V235D; V235F; V235L; L231E+V321T; L229I+I308A; I308A+A329V; I308A+G356F; I308A+G356P; I308A+G356Y; L229I+I308V; I308V+A329V; I308V+G356F; I308V+G356P; I308V+G356Y; G354A+G356V; G354A+G356Y; H310D+V355L; H310E+V355L; V311A+V355L; G354P+V355L; V355L+G356P; I184V+A192L; I184V+A192M; S143E; N361W; I308A+A330L; I308A+A330M; I308A+A330V; I308A+L333I; I308A+L333M; I308V+A330L; I308V+A330M; I308V+A330V; I308V+L333I; I308V+L333M; I308V+V355L; F318Y+V355L; H253E+D278R; K258Y+G391R; A36C+L85C; S100C+T317C; A179C+M223C; Y300C+L389C; A309C+A330C or equivalent amino acid substitutions thereof.

9. A polynucleotide, nucleic acid construct or expression vector encoding and/or capable of expressing one or more variants and/or polypeptides according to any one of the preceding claims.

10. A recombinant host cell (e.g., an isolated recombinant host cell), spore, transgenic plant, transgenic seed or transgenic pollen grain comprising one or more of the following: (i) one or more variants and/or polypeptides according to any one of the preceding claims; (ii) one or more polynucleotides according to any one of the preceding claims; and/or (iii) one or more nucleic acid constructs and/or expression vectors according to any one of the preceding claims.

11. A composition or kit comprising one or more of the following: variants, nucleic acid constructs, expression vectors, recombinant host cells, spores, transgenic plants, transgenic seeds and/or transgenic pollen grains, foodstuff, intermediate foodstuff; fodder, intermediate fodder; feed, intermediate feed; additive (e.g., foodstuff-, fodder- or feed additive), intermediate additive (e.g., foodstuff-, fodder- or feed intermediate additive); detoxifying agent, intermediate detoxifying agent; nutritional supplement, intermediate nutritional supplement, prebiotic, intermediate prebiotic and/or mixture/s thereof according to any one of the preceding claims and/or one or more polypeptides having at least 70% sequence identity (e.g. at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) to the amino acid sequence selected from the group consisting of: SEQ ID NOs: 1, 2, 16-21, 22, 23-34, 317-318, 326-335, 336-337, 348-349, 368-370, 35-316, 319-325, 338-347, 350-359; wherein said polypeptide/s are capable of detoxifying (e.g., modifying or hydrolyzing) at least one mycotoxin having said Formula I: 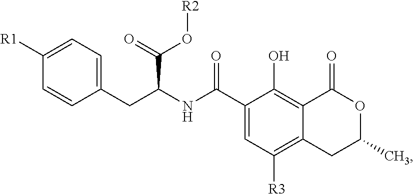
wherein R1 is selected from the group consisting of H and OH, R2 is selected from the group consisting of H and CH₂—CH₃ and R3 is selected from the group consisting of H and Cl; preferably said polypeptide/s having a peptidase activity having EC 3.4.13.X, wherein X is selected from the group consisting of: 9, 4, 5, 7, 12, 17, 18, 19, 20, 21, 22, 23; further preferably said polypeptide/s having a peptidase activity having EC 3.4.13.9; most preferably said polypeptide/s comprising one or more of the following amino acid sequences (e.g., conservative motifs):
i′) PGFIDAHVH (SEQ ID NO: 3), preferably said SEQ ID NO: 3 is comprised at amino acid positions corresponding to positions 87-95 of SEQ ID NO: 1 (e.g., using the numbering of SEQ ID NO: 1);
ii′) PAEQA (SEQ ID NO: 4), preferably said SEQ ID NO: 4 is comprised at amino acid positions corresponding to positions 116-120 of SEQ ID NO: 1 (e.g., using the numbering of SEQ ID NO: 1);
iii′) GVCNG (SEQ ID NO: 5), preferably said SEQ ID NO: 5 is comprised at amino acid positions corresponding to positions 197-201 of SEQ ID NO: 1 (e.g., using the numbering of SEQ ID NO: 1);
iv′) CRAAVR (SEQ ID NO: 6), preferably said SEQ ID NO: 6 is comprised at amino acid positions corresponding to positions 205-210 of SEQ ID NO: 1 (e.g., using the numbering of SEQ ID NO: 1);
v′) IKFMPSGGVLSL (SEQ ID NO: 7), preferably said SEQ ID NO: 7 is comprised at amino acid positions corresponding to positions 220-231 of SEQ ID NO: 1 (e.g., using the numbering of SEQ ID NO: 1);
vi′) RKVAAH (SEQ ID NO: 8), preferably said SEQ ID NO: 8 is comprised at amino acid positions corresponding to positions 257-262 of SEQ ID NO: 1 (e.g., using the numbering of SEQ ID NO: 1);
vii′) AGVDSIEHG (SEQ ID NO: 9), preferably said SEQ ID NO: 9 is comprised at amino acid positions corresponding to positions 275-283 of SEQ ID NO: 1 (e.g., using the numbering of SEQ ID NO: 1);
viii′) VMPGLIDAH (SEQ ID NO: 10), preferably said SEQ ID NO: 10 is comprised at amino acid positions corresponding to positions 51-59 of SEQ ID NO: 2 (e.g., using the numbering of SEQ ID NO: 2);
ix′) AGFTTVRDCG (SEQ ID NO: 11), preferably said SEQ ID NO: 11 is comprised at amino acid positions corresponding to positions 96-105 of SEQ ID NO: 2 (e.g., using the numbering of SEQ ID NO: 2);
x′) QTFGH (SEQ ID NO: 12), preferably said SEQ ID NO: 12 is comprised at amino acid positions corresponding to positions 135-139 of SEQ ID NO: 2 (e.g., using the numbering of SEQ ID NO: 2);
xi′) ATGGVLS (SEQ ID NO: 13), preferably said SEQ ID NO: 13 is comprised at amino acid positions corresponding to positions 185-191 of SEQ ID NO: 2 (e.g., using the numbering of SEQ ID NO: 2);
xii′) YVAAHAHGD (SEQ ID NO: 14), preferably said SEQ ID NO: 14 is comprised at amino acid positions corresponding to positions 219-227 of SEQ ID NO: 2 (e.g., using the numbering of SEQ ID NO: 2);
xiii′) LTPTL (SEQ ID NO: 15), preferably said SEQ ID NO: 15 is comprised at amino acid positions corresponding to positions 262-266 of SEQ ID NO: 2 (e.g., using the numbering of SEQ ID NO: 2);
xiv′) VMPGLID (SEQ ID NO: 360), preferably said SEQ ID NO: 360 is comprised at amino acid positions corresponding to positions 52-58 of SEQ ID NO: 22 (e.g., using the numbering of SEQ ID NO: 22);
xv′) KGGSL (SEQ ID NO: 361), preferably said SEQ ID NO: 361 is comprised at amino acid positions corresponding to positions 67-71 of SEQ ID NO: 22 (e.g., using the numbering of SEQ ID NO: 22);
xvi′) LLLAGFTTVRDCG (SEQ ID NO: 362), preferably said SEQ ID NO: 362 is comprised at amino acid positions corresponding to positions 93-105 of SEQ ID NO: 22 (e.g., using the numbering of SEQ ID NO: 22);
xvii′) QTFGHGE (SEQ ID NO: 363), preferably said SEQ ID NO: 363 is comprised at amino acid positions corresponding to positions 135-141 of SEQ ID NO: 22 (e.g., using the numbering of SEQ ID NO: 22);
xviii′) FATGGVLSQRD (SEQ ID NO: 364), preferably said SEQ ID NO: 364 is comprised at amino acid positions corresponding to positions 184-194 of SEQ ID NO: 22 (e.g., using the numbering of SEQ ID NO: 22);
xix′) IVNEA (SEQ ID NO: 365), preferably said SEQ ID NO: 365 is comprised at amino acid positions corresponding to positions 209-213 of SEQ ID NO: 22 (e.g., using the numbering of SEQ ID NO: 22);
xx′) IGVDEWGL (SEQ ID NO: 366), preferably said SEQ ID NO: 366 is comprised at amino acid positions corresponding to positions 279-286 of SEQ ID NO: 22 (e.g., using the numbering of SEQ ID NO: 22);
xxi′) GFETGL (SEQ ID NO: 367), preferably said SEQ ID NO: 367 is comprised at amino acid positions corresponding to positions 317-322 of SEQ ID NO: 22 (e.g., using the numbering of SEQ ID NO: 22).

12. A method for producing a foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent, intermediate detoxifying agent; nutritional supplement, intermediate nutritional supplement; prebiotic, intermediate prebiotic and/or mixture/s thereof, said method comprising: a) providing:
(i) one or more polypeptides having at least 70% sequence identity (e.g. at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) to the amino acid sequence selected from the group consisting of: SEQ ID NO: 1, SEQ ID NO: 2, SEQ ID NO: 22, SEQ ID NO: 318, SEQ ID NO: 336, SEQ ID NO: 348, SEQ ID NOs: 16-359, 368-370; wherein said polypeptide/s are capable of detoxifying (e.g., modifying or hydrolyzing) at least one mycotoxin having said Formula I (e.g., Ochratoxin A, Ochratoxin B and/or Ochratoxin C): 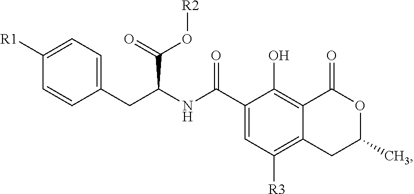
wherein R1 is selected from the group consisting of H and OH, R2 is selected from the group consisting of H and CH₂—CH₃ and R3 is selected from the group consisting of H and Cl; preferably said polypeptide/s having a peptidase activity having EC 3.4.13.X, wherein X is selected from the group consisting of: 9, 4, 5, 7, 12, 17, 18, 19, 20, 21, 22, 23; further preferably said polypeptide/s having a peptidase activity having EC 3.4.13.9; most preferably said polypeptide/s comprising one or more of the following amino acid sequences (e.g., conservative motifs):
i′) PGFIDAHVH (SEQ ID NO: 3), preferably said SEQ ID NO: 3 is comprised at amino acid positions corresponding to positions 87-95 of SEQ ID NO: 1 (e.g., using the numbering of SEQ ID NO: 1);
ii′) PAEQA (SEQ ID NO: 4), preferably said SEQ ID NO: 4 is comprised at amino acid positions corresponding to positions 116-120 of SEQ ID NO: 1 (e.g., using the numbering of SEQ ID NO: 1);
iii′) GVCNG (SEQ ID NO: 5), preferably said SEQ ID NO: 5 is comprised at amino acid positions corresponding to positions 197-201 of SEQ ID NO: 1 (e.g., using the numbering of SEQ ID NO: 1);
iv′) CRAAVR (SEQ ID NO: 6), preferably said SEQ ID NO: 6 is comprised at amino acid positions corresponding to positions 205-210 of SEQ ID NO: 1 (e.g., using the numbering of SEQ ID NO: 1);
v′) IKFMPSGGVLSL (SEQ ID NO: 7), preferably said SEQ ID NO: 7 is comprised at amino acid positions corresponding to positions 220-231 of SEQ ID NO: 1 (e.g., using the numbering of SEQ ID NO: 1);
vi′) RKVAAH (SEQ ID NO: 8), preferably said SEQ ID NO: 8 is comprised at amino acid positions corresponding to positions 257-262 of SEQ ID NO: 1 (e.g., using the numbering of SEQ ID NO: 1);
vii′) AGVDSIEHG (SEQ ID NO: 9), preferably said SEQ ID NO: 9 is comprised at amino acid positions corresponding to positions 275-283 of SEQ ID NO: 1 (e.g., using the numbering of SEQ ID NO: 1);
viii′) VMPGLIDAH (SEQ ID NO: 10), preferably said SEQ ID NO: 10 is comprised at amino acid positions corresponding to positions 51-59 of SEQ ID NO: 2 (e.g., using the numbering of SEQ ID NO: 2);
ix′) AGFTTVRDCG (SEQ ID NO: 11), preferably said SEQ ID NO: 11 is comprised at amino acid positions corresponding to positions 96-105 of SEQ ID NO: 2 (e.g., using the numbering of SEQ ID NO: 2);
x′) QTFGH (SEQ ID NO: 12), preferably said SEQ ID NO: 12 is comprised at amino acid positions corresponding to positions 135-139 of SEQ ID NO: 2 (e.g., using the numbering of SEQ ID NO: 2);
xi′) ATGGVLS (SEQ ID NO: 13), preferably said SEQ ID NO: 13 is comprised at amino acid positions corresponding to positions 185-191 of SEQ ID NO: 2 (e.g., using the numbering of SEQ ID NO: 2);
xii′) YVAAHAHGD (SEQ ID NO: 14), preferably said SEQ ID NO: 14 is comprised at amino acid positions corresponding to positions 219-227 of SEQ ID NO: 2 (e.g., using the numbering of SEQ ID NO: 2);
xiii′) LTPTL (SEQ ID NO: 15), preferably said SEQ ID NO: 15 is comprised at amino acid positions corresponding to positions 262-266 of SEQ ID NO: 2 (e.g., using the numbering of SEQ ID NO: 2);
xiv′) VMPGLID (SEQ ID NO: 360), preferably said SEQ ID NO: 360 is comprised at amino acid positions corresponding to positions 52-58 of SEQ ID NO: 22 (e.g., using the numbering of SEQ ID NO: 22);
xv′) KGGSL (SEQ ID NO: 361), preferably said SEQ ID NO: 361 is comprised at amino acid positions corresponding to positions 67-71 of SEQ ID NO: 22 (e.g., using the numbering of SEQ ID NO: 22);
xvi′) LLLAGFTTVRDCG (SEQ ID NO: 362), preferably said SEQ ID NO: 362 is comprised at amino acid positions corresponding to positions 93-105 of SEQ ID NO: 22 (e.g., using the numbering of SEQ ID NO: 22);
xvii′) QTFGHGE (SEQ ID NO: 363), preferably said SEQ ID NO: 363 is comprised at amino acid positions corresponding to positions 135-141 of SEQ ID NO: 22 (e.g., using the numbering of SEQ ID NO: 22);
xviii′) FATGGVLSQRD (SEQ ID NO: 364), preferably said SEQ ID NO: 364 is comprised at amino acid positions corresponding to positions 184-194 of SEQ ID NO: 22 (e.g., using the numbering of SEQ ID NO: 22);
xix′) IVNEA (SEQ ID NO: 365), preferably said SEQ ID NO: 365 is comprised at amino acid positions corresponding to positions 209-213 of SEQ ID NO: 22 (e.g., using the numbering of SEQ ID NO: 22);
xx′) IGVDEWGL (SEQ ID NO: 366), preferably said SEQ ID NO: 366 is comprised at amino acid positions corresponding to positions 279-286 of SEQ ID NO: 22 (e.g., using the numbering of SEQ ID NO: 22);
xxi′) GFETGL (SEQ ID NO: 367), preferably said SEQ ID NO: 367 is comprised at amino acid positions corresponding to positions 317-322 of SEQ ID NO: 22 (e.g., using the numbering of SEQ ID NO: 22),
(ii) variants according to any one of the preceding claims;
(iii) one or more polynucleotides according to any one of the preceding claims;
(iv) one or more nucleic acid constructs and/or expression vectors according to any one of the preceding claims; and/or
(v) one or more recombinant host cells, spores, transgenic plants, transgenic seeds and/or transgenic pollen grains according to any one of the preceding claims; preferably said one or more polypeptides are one or more recombinant and/or isolated polypeptides;
b) applying (a) to a nutritive source or material suitable for production of foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent, intermediate detoxifying agent;
nutritional supplement, intermediate nutritional supplement; prebiotic, intermediate prebiotic and/or mixture/s thereof.

13. A foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent, intermediate detoxifying agent; nutritional supplement, intermediate nutritional supplement; prebiotic, intermediate prebiotic and/or mixture/s thereof produced by the method according to any one of the preceding claims.

14. The variant, polynucleotide, nucleic acid construct, expression vector, recombinant host cell, spore, transgenic plant, transgenic seed, transgenic pollen grain, foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent, intermediate detoxifying agent; nutritional supplement, intermediate nutritional supplement; prebiotic, intermediate prebiotic or mixture/s thereof, composition or kit according to any one of the preceding claims and/or one or more polypeptides having at least 70% sequence identity (e.g. at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) to the amino acid sequence selected from the group consisting of: SEQ ID NO: 1, SEQ ID NO: 2, SEQ ID NO: 22, SEQ ID NO: 318, SEQ ID NO: 336, SEQ ID NO: 348, SEQ ID NOs: 16-359, 368-370; wherein said polypeptide/s are capable of detoxifying (e.g., modifying or hydrolyzing) at least one mycotoxin having said Formula I (e.g., Ochratoxin A, Ochratoxin B and/or Ochratoxin C): 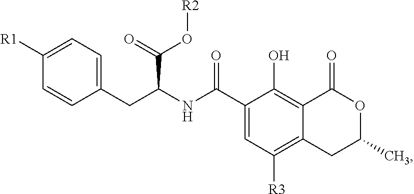
wherein R1 is selected from the group consisting of H and OH, R2 is selected from the group consisting of H and CH₂—CH₃ and R3 is selected from the group consisting of H and Cl; preferably said polypeptide/s having a peptidase activity having EC 3.4.13.X, wherein X is selected from the group consisting of: 9, 4, 5, 7, 12, 17, 18, 19, 20, 21, 22, 23; further preferably said polypeptide/s having a peptidase activity having EC 3.4.13.9; most preferably said polypeptide/s comprising one or more of the following amino acid sequences (e.g., conservative motifs):
i′) PGFIDAHVH (SEQ ID NO: 3), preferably said SEQ ID NO: 3 is comprised at amino acid positions corresponding to positions 87-95 of SEQ ID NO: 1 (e.g., using the numbering of SEQ ID NO: 1);
ii′) PAEQA (SEQ ID NO: 4), preferably said SEQ ID NO: 4 is comprised at amino acid positions corresponding to positions 116-120 of SEQ ID NO: 1 (e.g., using the numbering of SEQ ID NO: 1);
iii′) GVCNG (SEQ ID NO: 5), preferably said SEQ ID NO: 5 is comprised at amino acid positions corresponding to positions 197-201 of SEQ ID NO: 1 (e.g., using the numbering of SEQ ID NO: 1);
iv′) CRAAVR (SEQ ID NO: 6), preferably said SEQ ID NO: 6 is comprised at amino acid positions corresponding to positions 205-210 of SEQ ID NO: 1 (e.g., using the numbering of SEQ ID NO: 1);
v′) IKFMPSGGVLSL (SEQ ID NO: 7), preferably said SEQ ID NO: 7 is comprised at amino acid positions corresponding to positions 220-231 of SEQ ID NO: 1 (e.g., using the numbering of SEQ ID NO: 1);
vi′) RKVAAH (SEQ ID NO: 8), preferably said SEQ ID NO: 8 is comprised at amino acid positions corresponding to positions 257-262 of SEQ ID NO: 1 (e.g., using the numbering of SEQ ID NO: 1);
vii′) AGVDSIEHG (SEQ ID NO: 9), preferably said SEQ ID NO: 9 is comprised at amino acid positions corresponding to positions 275-283 of SEQ ID NO: 1 (e.g., using the numbering of SEQ ID NO: 1);
viii′) VMPGLIDAH (SEQ ID NO: 10), preferably said SEQ ID NO: 10 is comprised at amino acid positions corresponding to positions 51-59 of SEQ ID NO: 2 (e.g., using the numbering of SEQ ID NO: 2);
ix′) AGFTTVRDCG (SEQ ID NO: 11), preferably said SEQ ID NO: 11 is comprised at amino acid positions corresponding to positions 96-105 of SEQ ID NO: 2 (e.g., using the numbering of SEQ ID NO: 2);
x′) QTFGH (SEQ ID NO: 12), preferably said SEQ ID NO: 12 is comprised at amino acid positions corresponding to positions 135-139 of SEQ ID NO: 2 (e.g., using the numbering of SEQ ID NO: 2);
xi′) ATGGVLS (SEQ ID NO: 13), preferably said SEQ ID NO: 13 is comprised at amino acid positions corresponding to positions 185-191 of SEQ ID NO: 2 (e.g., using the numbering of SEQ ID NO: 2);
xii′) YVAAHAHGD (SEQ ID NO: 14), preferably said SEQ ID NO: 14 is comprised at amino acid positions corresponding to positions 219-227 of SEQ ID NO: 2 (e.g., using the numbering of SEQ ID NO: 2);
xiii′) LTPTL (SEQ ID NO: 15), preferably said SEQ ID NO: 15 is comprised at amino acid positions corresponding to positions 262-266 of SEQ ID NO: 2 (e.g., using the numbering of SEQ ID NO: 2);
xiv′) VMPGLID (SEQ ID NO: 360), preferably said SEQ ID NO: 360 is comprised at amino acid positions corresponding to positions 52-58 of SEQ ID NO: 22 (e.g., using the numbering of SEQ ID NO: 22);
xv′) KGGSL (SEQ ID NO: 361), preferably said SEQ ID NO: 361 is comprised at amino acid positions corresponding to positions 67-71 of SEQ ID NO: 22 (e.g., using the numbering of SEQ ID NO: 22);
xvi′) LLLAGFTTVRDCG (SEQ ID NO: 362), preferably said SEQ ID NO: 362 is comprised at amino acid positions corresponding to positions 93-105 of SEQ ID NO: 22 (e.g., using the numbering of SEQ ID NO: 22);
xvii′) QTFGHGE (SEQ ID NO: 363), preferably said SEQ ID NO: 363 is comprised at amino acid positions corresponding to positions 135-141 of SEQ ID NO: 22 (e.g., using the numbering of SEQ ID NO: 22);
xviii′) FATGGVLSQRD (SEQ ID NO: 364), preferably said SEQ ID NO: 364 is comprised at amino acid positions corresponding to positions 184-194 of SEQ ID NO: 22 (e.g., using the numbering of SEQ ID NO: 22);
xix′) IVNEA (SEQ ID NO: 365), preferably said SEQ ID NO: 365 is comprised at amino acid positions corresponding to positions 209-213 of SEQ ID NO: 22 (e.g., using the numbering of SEQ ID NO: 22);
xx′) IGVDEWGL (SEQ ID NO: 366), preferably said SEQ ID NO: 366 is comprised at amino acid positions corresponding to positions 279-286 of SEQ ID NO: 22 (e.g., using the numbering of SEQ ID NO: 22);
xxi′) GFETGL (SEQ ID NO: 367), preferably said SEQ ID NO: 367 is comprised at amino acid positions corresponding to positions 317-322 of SEQ ID NO: 22 (e.g., using the numbering of SEQ ID NO: 22),
for use as a medicament (e.g., for veterinary use) and/or in therapy and/or prophylaxis of a disease.

15. The variant, polynucleotide, nucleic acid construct, expression vector, recombinant host cell, spore, transgenic plant, transgenic seed, transgenic pollen grain, foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent, intermediate detoxifying agent; nutritional supplement, intermediate nutritional supplement; prebiotic, intermediate prebiotic or mixture/s thereof, composition or kit according to any one of the preceding claims and/or one or more polypeptides having at least 70% sequence identity (e.g. at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) to the amino acid sequence selected from the group consisting of: SEQ ID NO: 1, SEQ ID NO: 2, SEQ ID NO: 22, SEQ ID NO: 318, SEQ ID NO: 336, SEQ ID NO: 348, SEQ ID NOs: 16-359, 368-370; wherein said polypeptide/s are capable of detoxifying (e.g., modifying or hydrolyzing) at least one mycotoxin having said Formula I (e.g., Ochratoxin A, Ochratoxin B and/or Ochratoxin C): 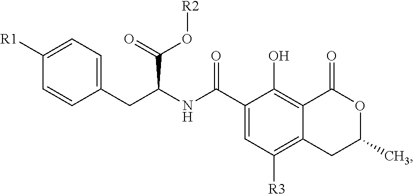
wherein R1 is selected from the group consisting of H and OH, R2 is selected from the group consisting of H and CH₂—CH₃ and R3 is selected from the group consisting of H and Cl; preferably said polypeptide/s having a peptidase activity having EC 3.4.13.X, wherein X is selected from the group consisting of: 9, 4, 5, 7, 12, 17, 18, 19, 20, 21, 22, 23; further preferably said polypeptide/s having a peptidase activity having EC 3.4.13.9; most preferably said polypeptide/s comprising one or more of the following amino acid sequences (e.g., conservative motifs):
i′) PGFIDAHVH (SEQ ID NO: 3), preferably said SEQ ID NO: 3 is comprised at amino acid positions corresponding to positions 87-95 of SEQ ID NO: 1 (e.g., using the numbering of SEQ ID NO: 1);
ii′) PAEQA (SEQ ID NO: 4), preferably said SEQ ID NO: 4 is comprised at amino acid positions corresponding to positions 116-120 of SEQ ID NO: 1 (e.g., using the numbering of SEQ ID NO: 1);
iii′) GVCNG (SEQ ID NO: 5), preferably said SEQ ID NO: 5 is comprised at amino acid positions corresponding to positions 197-201 of SEQ ID NO: 1 (e.g., using the numbering of SEQ ID NO: 1);
iv′) CRAAVR (SEQ ID NO: 6), preferably said SEQ ID NO: 6 is comprised at amino acid positions corresponding to positions 205-210 of SEQ ID NO: 1 (e.g., using the numbering of SEQ ID NO: 1);
v′) IKFMPSGGVLSL (SEQ ID NO: 7), preferably said SEQ ID NO: 7 is comprised at amino acid positions corresponding to positions 220-231 of SEQ ID NO: 1 (e.g., using the numbering of SEQ ID NO: 1);
vi′) RKVAAH (SEQ ID NO: 8), preferably said SEQ ID NO: 8 is comprised at amino acid positions corresponding to positions 257-262 of SEQ ID NO: 1 (e.g., using the numbering of SEQ ID NO: 1);
vii′) AGVDSIEHG (SEQ ID NO: 9), preferably said SEQ ID NO: 9 is comprised at amino acid positions corresponding to positions 275-283 of SEQ ID NO: 1 (e.g., using the numbering of SEQ ID NO: 1);
viii′) VMPGLIDAH (SEQ ID NO: 10), preferably said SEQ ID NO: 10 is comprised at amino acid positions corresponding to positions 51-59 of SEQ ID NO: 2 (e.g., using the numbering of SEQ ID NO: 2);
ix′) AGFTTVRDCG (SEQ ID NO: 11), preferably said SEQ ID NO: 11 is comprised at amino acid positions corresponding to positions 96-105 of SEQ ID NO: 2 (e.g., using the numbering of SEQ ID NO: 2);
x′) QTFGH (SEQ ID NO: 12), preferably said SEQ ID NO: 12 is comprised at amino acid positions corresponding to positions 135-139 of SEQ ID NO: 2 (e.g., using the numbering of SEQ ID NO: 2);
xi′) ATGGVLS (SEQ ID NO: 13), preferably said SEQ ID NO: 13 is comprised at amino acid positions corresponding to positions 185-191 of SEQ ID NO: 2 (e.g., using the numbering of SEQ ID NO: 2);
xii′) YVAAHAHGD (SEQ ID NO: 14), preferably said SEQ ID NO: 14 is comprised at amino acid positions corresponding to positions 219-227 of SEQ ID NO: 2 (e.g., using the numbering of SEQ ID NO: 2);
xiii′) LTPTL (SEQ ID NO: 15), preferably said SEQ ID NO: 15 is comprised at amino acid positions corresponding to positions 262-266 of SEQ ID NO: 2 (e.g., using the numbering of SEQ ID NO: 2);
xiv′) VMPGLID (SEQ ID NO: 360), preferably said SEQ ID NO: 360 is comprised at amino acid positions corresponding to positions 52-58 of SEQ ID NO: 22 (e.g., using the numbering of SEQ ID NO: 22);
xv′) KGGSL (SEQ ID NO: 361), preferably said SEQ ID NO: 361 is comprised at amino acid positions corresponding to positions 67-71 of SEQ ID NO: 22 (e.g., using the numbering of SEQ ID NO: 22);
xvi′) LLLAGFTTVRDCG (SEQ ID NO: 362), preferably said SEQ ID NO: 362 is comprised at amino acid positions corresponding to positions 93-105 of SEQ ID NO: 22 (e.g., using the numbering of SEQ ID NO: 22);
xvii′) QTFGHGE (SEQ ID NO: 363), preferably said SEQ ID NO: 363 is comprised at amino acid positions corresponding to positions 135-141 of SEQ ID NO: 22 (e.g., using the numbering of SEQ ID NO: 22);
xviii′) FATGGVLSQRD (SEQ ID NO: 364), preferably said SEQ ID NO: 364 is comprised at amino acid positions corresponding to positions 184-194 of SEQ ID NO: 22 (e.g., using the numbering of SEQ ID NO: 22);
xix′) IVNEA (SEQ ID NO: 365), preferably said SEQ ID NO: 365 is comprised at amino acid positions corresponding to positions 209-213 of SEQ ID NO: 22 (e.g., using the numbering of SEQ ID NO: 22);
xx′) IGVDEWGL (SEQ ID NO: 366), preferably said SEQ ID NO: 366 is comprised at amino acid positions corresponding to positions 279-286 of SEQ ID NO: 22 (e.g., using the numbering of SEQ ID NO: 22);
xxi′) GFETGL (SEQ ID NO: 367), preferably said SEQ ID NO: 367 is comprised at amino acid positions corresponding to positions 317-322 of SEQ ID NO: 22 (e.g., using the numbering of SEQ ID NO: 22),
for use in one or more of the following methods:
i) method for treatment, amelioration, prophylaxis and/or diagnostics of mycotoxicosis, preferably an OTA mycotoxicosis;
ii) method for monitoring development of mycotoxicosis and/or assessing the efficacy of a mycotoxicosis prophylaxis and/or therapy, preferably an OTA mycotoxicosis prophylaxis and/or therapy;
iii) method for detoxifying and/or altering toxicity of a mycotoxin having Formula I;
iv) method of producing one or more of the following: foodstuff, intermediate foodstuff; fodder, intermediate fodder; feed intermediate feed; additive (e.g., foodstuff-, fodder- or feed additive), intermediate additive (e.g., foodstuff-, fodder- or feed intermediate additive); detoxifying agent, intermediate detoxifying agent; nutritional supplement, intermediate nutritional supplement; prebiotic, intermediate prebiotic; pharmaceutical, veterinary, diagnostic, detoxifying, monitoring and/or screening composition or kit;
v) method according to any one of the preceding claims;
vi) any combination of methods according to (i)-(v);
vii) method of any of (i)-(vi), wherein said method is an in vitro, ex vivo, in vivo and/or manufacturing method.

16. Use of one or more of variants, polynucleotides, nucleic acid constructs, expression vectors, recombinant host cells, spores, transgenic plants, transgenic seeds, transgenic pollen grains, foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent, intermediate detoxifying agent; nutritional supplement, intermediate nutritional supplement; prebiotic, intermediate prebiotic or mixture/s thereof, composition or kit according to any one of the preceding claims and/or one or more polypeptides having at least 70% sequence identity (e.g. at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) to the amino acid sequence selected from the group consisting of: SEQ ID NO: 1, SEQ ID NO: 2, SEQ ID NO: 22, SEQ ID NO: 318, SEQ ID NO: 336, SEQ ID NO: 348, SEQ ID NOs: 16-359, 368-370; wherein said polypeptide/s are capable of detoxifying (e.g., modifying or hydrolyzing) at least one mycotoxin having said Formula I (e.g., Ochratoxin A, Ochratoxin B and/or Ochratoxin C): 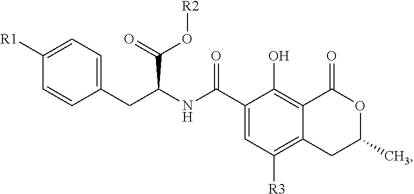
wherein R1 is selected from the group consisting of H and OH, R2 is selected from the group consisting of H and CH₂—CH₃ and R3 is selected from the group consisting of H and Cl; preferably said polypeptide/s having a peptidase activity having EC 3.4.13.X, wherein X is selected from the group consisting of: 9, 4, 5, 7, 12, 17, 18, 19, 20, 21, 22, 23; further preferably said polypeptide/s having a peptidase activity having EC 3.4.13.9; most preferably said polypeptide/s comprising one or more of the following amino acid sequences (e.g., conservative motifs):
i′) PGFIDAHVH (SEQ ID NO: 3), preferably said SEQ ID NO: 3 is comprised at amino acid positions corresponding to positions 87-95 of SEQ ID NO: 1 (e.g., using the numbering of SEQ ID NO: 1);
ii′) PAEQA (SEQ ID NO: 4), preferably said SEQ ID NO: 4 is comprised at amino acid positions corresponding to positions 116-120 of SEQ ID NO: 1 (e.g., using the numbering of SEQ ID NO: 1);
iii′) GVCNG (SEQ ID NO: 5), preferably said SEQ ID NO: 5 is comprised at amino acid positions corresponding to positions 197-201 of SEQ ID NO: 1 (e.g., using the numbering of SEQ ID NO: 1);
iv′) CRAAVR (SEQ ID NO: 6), preferably said SEQ ID NO: 6 is comprised at amino acid positions corresponding to positions 205-210 of SEQ ID NO: 1 (e.g., using the numbering of SEQ ID NO: 1);
v′) IKFMPSGGVLSL (SEQ ID NO: 7), preferably said SEQ ID NO: 7 is comprised at amino acid positions corresponding to positions 220-231 of SEQ ID NO: 1 (e.g., using the numbering of SEQ ID NO: 1);
vi′) RKVAAH (SEQ ID NO: 8), preferably said SEQ ID NO: 8 is comprised at amino acid positions corresponding to positions 257-262 of SEQ ID NO: 1 (e.g., using the numbering of SEQ ID NO: 1);
vii′) AGVDSIEHG (SEQ ID NO: 9), preferably said SEQ ID NO: 9 is comprised at amino acid positions corresponding to positions 275-283 of SEQ ID NO: 1 (e.g., using the numbering of SEQ ID NO: 1);
viii′) VMPGLIDAH (SEQ ID NO: 10), preferably said SEQ ID NO: 10 is comprised at amino acid positions corresponding to positions 51-59 of SEQ ID NO: 2 (e.g., using the numbering of SEQ ID NO: 2);
ix′) AGFTTVRDCG (SEQ ID NO: 11), preferably said SEQ ID NO: 11 is comprised at amino acid positions corresponding to positions 96-105 of SEQ ID NO: 2 (e.g., using the numbering of SEQ ID NO: 2);
x′) QTFGH (SEQ ID NO: 12), preferably said SEQ ID NO: 12 is comprised at amino acid positions corresponding to positions 135-139 of SEQ ID NO: 2 (e.g., using the numbering of SEQ ID NO: 2);
xi′) ATGGVLS (SEQ ID NO: 13), preferably said SEQ ID NO: 13 is comprised at amino acid positions corresponding to positions 185-191 of SEQ ID NO: 2 (e.g., using the numbering of SEQ ID NO: 2);
xii′) YVAAHAHGD (SEQ ID NO: 14), preferably said SEQ ID NO: 14 is comprised at amino acid positions corresponding to positions 219-227 of SEQ ID NO: 2 (e.g., using the numbering of SEQ ID NO: 2);
xiii′) LTPTL (SEQ ID NO: 15), preferably said SEQ ID NO: 15 is comprised at amino acid positions corresponding to positions 262-266 of SEQ ID NO: 2 (e.g., using the numbering of SEQ ID NO: 2);
xiv′) VMPGLID (SEQ ID NO: 360), preferably said SEQ ID NO: 360 is comprised at amino acid positions corresponding to positions 52-58 of SEQ ID NO: 22 (e.g., using the numbering of SEQ ID NO: 22);
xv′) KGGSL (SEQ ID NO: 361), preferably said SEQ ID NO: 361 is comprised at amino acid positions corresponding to positions 67-71 of SEQ ID NO: 22 (e.g., using the numbering of SEQ ID NO: 22);
xvi′) LLLAGFTTVRDCG (SEQ ID NO: 362), preferably said SEQ ID NO: 362 is comprised at amino acid positions corresponding to positions 93-105 of SEQ ID NO: 22 (e.g., using the numbering of SEQ ID NO: 22);
xvii′) QTFGHGE (SEQ ID NO: 363), preferably said SEQ ID NO: 363 is comprised at amino acid positions corresponding to positions 135-141 of SEQ ID NO: 22 (e.g., using the numbering of SEQ ID NO: 22);
xviii′) FATGGVLSQRD (SEQ ID NO: 364), preferably said SEQ ID NO: 364 is comprised at amino acid positions corresponding to positions 184-194 of SEQ ID NO: 22 (e.g., using the numbering of SEQ ID NO: 22);
xix′) IVNEA (SEQ ID NO: 365), preferably said SEQ ID NO: 365 is comprised at amino acid positions corresponding to positions 209-213 of SEQ ID NO: 22 (e.g., using the numbering of SEQ ID NO: 22);
xx′) IGVDEWGL (SEQ ID NO: 366), preferably said SEQ ID NO: 366 is comprised at amino acid positions corresponding to positions 279-286 of SEQ ID NO: 22 (e.g., using the numbering of SEQ ID NO: 22);
xxi′) GFETGL (SEQ ID NO: 367), preferably said SEQ ID NO: 367 is comprised at amino acid positions corresponding to positions 317-322 of SEQ ID NO: 22 (e.g., using the numbering of SEQ ID NO: 22),
for/in one or more of the following:
i) treatment, amelioration, prophylaxis and/or diagnostics of mycotoxicosis, preferably OTA mycotoxicosis;
ii) monitoring development of mycotoxicosis and/or assessing the efficacy of a mycotoxicosis prophylaxis and/or therapy, preferably OTA mycotoxicosis prophylaxis and/or therapy;
iii) detoxifying and/or altering toxicity of a mycotoxin having Formula I;
iv) producing one or more of the following: foodstuff, intermediate foodstuff; fodder, intermediate fodder; feed intermediate feed; additive (e.g., foodstuff-, fodder- or feed additive), intermediate additive (e.g., foodstuff-, fodder- or feed intermediate additive); detoxifying agent, intermediate detoxifying agent; nutritional supplement, intermediate nutritional supplement; prebiotic, intermediate prebiotic; pharmaceutical, veterinary, diagnostic, detoxifying, monitoring and/or screening composition or kit;
v) in method according to any one of the preceding claims;
vi) any combination of according to (i)-(v);
vii) use of any of (i)-(vi), wherein said use is an in vitro, ex vivo, in vivo use and/or use in a manufacturing method.


SEQUENCE LISTING (370 sequences)

SEQ ID NO: 1 - protein, 416 aa, synthetic construct
  REGION 1..416: note = polypeptide 1
APAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 2 - protein, 408 aa, synthetic construct
  REGION 1..408: note = polypeptide 2
MYSLAIRNGIVFDGEKFIGKTNIYVDGDKIVKITNERLDAKEEVNAEGYFVMPGLIDAHLHLTGIRGGSLLMGYLLDDPRTRLLRAANWARELLMAGFTTVRDCGEPNSIILRNAINEGIIPGPRIIAAGSPITQTFGHAELSHSLPLEWSIQLGFGEICDGVDECIKAARKVLRSGADFIKIMATGGVLSERDRPEWPQLTPEEMSAIVREAEKVGTYVAAHAHGDKGARIAVEAGVRTIEHGSLITEETVKLMAEKGVSLTPTLSIQELIYKYGKQMGIDPWGLEKIGQVREGVINSIKLARKYGVMILAGTDLGFKTGLDIDMGKNWVEVKLLVELGGLSSEEALRAATGNASKMLRLNTGIIREGYLADIVLINGNPLENINDIARVSTVIKGGKVTVENGKLI

SEQ ID NO: 3 - protein, 9 aa, synthetic construct
  REGION 1..9: note = conservative motif
PGFIDAHVH

SEQ ID NO: 4 - protein, 5 aa, synthetic construct
  REGION 1..5: note = conservative motif
PAEQA

SEQ ID NO: 5 - protein, 5 aa, synthetic construct
  REGION 1..5: note = conservative motif
GVCNG

SEQ ID NO: 6 - protein, 6 aa, synthetic construct
  REGION 1..6: note = conservative motif
CRAAVR

SEQ ID NO: 7 - protein, 12 aa, synthetic construct
  REGION 1..12: note = conservative motif
IKFMPSGGVLSL

SEQ ID NO: 8 - protein, 6 aa, synthetic construct
  REGION 1..6: note = conservative motif
RKVAAH

SEQ ID NO: 9 - protein, 9 aa, synthetic construct
  REGION 1..9: note = conservative motif
AGVDSIEHG

SEQ ID NO: 10 - protein, 9 aa, synthetic construct
  REGION 1..9: note = conservative motif
VMPGLIDAH

SEQ ID NO: 11 - protein, 10 aa, synthetic construct
  REGION 1..10: note = conservative motif
AGFTTVRDCG

SEQ ID NO: 12 - protein, 5 aa, synthetic construct
  REGION 1..5: note = conservative motif
QTFGH

SEQ ID NO: 13 - protein, 7 aa, synthetic construct
  REGION 1..7: note = conservative motif
ATGGVLS

SEQ ID NO: 14 - protein, 9 aa, synthetic construct
  REGION 1..9: note = conservative motif
YVAAHAHGD

SEQ ID NO: 15 - protein, 5 aa, synthetic construct
  REGION 1..5: note = conservative motif
LTPTL

SEQ ID NO: 16 - protein, 451 aa, synthetic construct
  REGION 1..451: note = exemplary variant
MGFSNDGLRHDRFRRTPHPPRRAHPGRRRARPWRRPGPGPQAAPHTVLLRAAHVFDSVSGRLIDNAQVLVVGNKIARVGANLSAPAGAETIDLGDATLLPGFIDAHVHLSGQSSDNWYGDFFDNMMRFPAEQALYGAYNARITLEAGFTSVRDVGSTDWIALGLRNAIDAGKIPGPRMLVSNYAVGSTGGHADQDPVPPQKIAVAGPIQGVCNGPDECRAAVRYQIKYGANVIKFMPSGGVLSLSDPVDAPELTQDEMNAIIGEAHAWGRKVAAHAHGDKAAKMAVQAGVDSIEHGSFLEDDTLRLMKQKGVFLVPTISAVAHVEEKIATFPPVIADKGRAAANHLRTTFRHAAQIGVPIALGTDAAVGPHGANAREFNFMVQNGLTPAQALMAGTCNGARLLGTIDKVGTLQEGKLADIVAVPGNPLTDIQATERPLFVMKDGVVVKRAR

SEQ ID NO: 17 - protein, 444 aa, synthetic construct
  REGION 1..444: note = polypeptide 3
MTTRLRILTASLALLAANACAADAAPSTPHTVVLRAARMFDGASGKIVEHPQLTIVGNKIVRIAQGKEEDNRTGIPAGADVIDLGDATLLPGFIDAHVHLSGQSSDNWYGDFFDNMMRFPAEQALYGAYYARITLEAGFTSVRDVGSTDWIALGLRNAIDAGKVPGPRMLVSNYAVGSTGGHADQDPIPPQKLAPAGPLQGVCNGPEECRAAVRYQIKYGANVIKFMPSGGVLSLSDPVDAPELSQDEMNAVIGEAHAWGRKVAAHAHGDKAAKMAVTAGVDSIEHGSFLEDDTLRMMKAKGVYLVPTISAIAHVEEKIATFPSVIAEKGRAAASHSRQMFRHAVQIGVPIALGTDAGVGAHGTNAREFRYMVDNGLTPAQSLMAGTANAARLLGTFDRVGTLEAGKLADVVAVPGNPIADITVTERPVFVMKDGVVVRRGAVR

SEQ ID NO: 18 - protein, 443 aa, synthetic construct
  REGION 1..443: note = polypeptide 4
MPPMRTRTRILTASLALLAANAWAADAMPSTPHTVVLRAARMFDGASGKLVEHPQLTIVGNKIVRVAQDDKAAVPAGADVIDLGDATLLPGFIDAHVHLSGQSSDNWYGDFFDNMMRFPAEQALYGAWYARITLEAGFTSVRDVGSTDWIALGLRNAIDAGKVPGPRMLVSNYAIGSTGGHADQDPIPPQKLAPAGPLQGVCNGPDECRAAVRYQIKYGANVIKFMPSGGVLSLSDPVDAPELTQEEMNAVVGEAHAWGRKVAAHAHGDKAAKMAVAAGVDSIEHGSFLEDDTLRMMKAKGVYLVPTISAVMHVEEKIATFPPVIAEKGRAAAAHVRQTFRHAVQIGVPIALGTDAGVGAHGTNAREFRYMVDNGMAPAQALLAGTSNAARLLGTIDRVGTLEAGKLADVVAVPGNPVADITVTERPVFVMKDGVVVRRGAVR

SEQ ID NO: 19 - protein, 430 aa, synthetic construct
  REGION 1..430: note = polypeptide 5
MLSLLAANACVADAAPAASRTIVLRAARMFDGASGKLVEHPQLTIVGNKIVRIAQSDNAEVPASAEVIDLGDATLLPGFIDAHVHLSGQSSDNWYGDFFDNMMRFPAEQALYGAYYACITLEAGFTSVRDLGSTDWVALGLRNAIDAGKVPGPRMLVSNYAVGSTGGHADQDPIPPHRLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIATFPPVIAEKGRAAAAHSRQTFRHAVQIGVPIALGTDAGVGAHGTNAREFRYMVENGLTPVQSLLAGTANAARLLGIIDRVGTLEAGKLADIVAVPGNPVADITVTERPVFVMKDGVVVRRGAVR

SEQ ID NO: 20 - protein, 440 aa, synthetic construct
  REGION 1..440: note = polypeptide 6
MTTRFRILTAVLSLLAASACAADAAPATPHTVVLRAARMFDGASGKLVERPQLTIVGNKIVRIAQADKAEVPAGAEVIDLGDSTLLPGFIDAHVHLSGQGSDNWYGDFFDNMMRFPAEQALYGAYYARITLEAGFTSVRDLGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGTNAREFRYMVENGLTPAQSLLAGTSNAARLLGTIDKVGTLEAGKLADVVAVPGNPIADITVTERPVFVMKDGVVVRRGAVR

SEQ ID NO: 21 - protein, 440 aa, synthetic construct
  REGION 1..440: note = polypeptide 7
MTTPTRILTAVLALLASNACAADATPSTPHTVVLRAARMFDGASGKLVERPQLTIVGNKIVRIAQADKADVPAGAEVIDLGDATLLPGFIDAHVHLSGQGSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKGVYLVPTISAIAHVEEKIATFPPVIAEKGRAAAAHLRQTFRHAVQIGVPIALGTDAGVGAHGTNAREFRYMVENGLSPAQSLLAGTSNAARLLGTIDRVGTLEAGKLADVVAVPGNPIADITVTERPVFVMKDGVVVRRGAVR

SEQ ID NO: 22 - protein, 412 aa, synthetic construct
  REGION 1..412: note = polypeptide 8
MQYDVAIKNGNVFTGTEFIGKMNIYISNGKISKVSKEDLNAKQEIDAKDMLVMPGLIDAHMHISGLKGGSLLKIMFEKPEYRALRATKWLEKLLLAGFTTVRDCGEPISISLKRAVNEGYIKGPKIIAAGKPITQTFGHGEFSHDIPLEFSISLGFSEICDGTESCIHAARKVLRDGSDFIKIFATGGVLSQRDRPDMPQLSFDEIRAIVNEAEKAGTYVAAHAHGDKGARIAIEAGIKTLEHGSLLKEDTLKLMLEKNISLTPTLTITEIIYKYGKTIGVDEWGLEKIVEVRENLHKVLRKAKELGITIITGTDMGFETGLEDYDIGKNWTEILLLTEVGGLSNEEALRAATYNSAKALGLNSGLIDEGKDADIIVIDGNPLENIKDVSKVVHVIKQGKLEVLDGKIINNY

SEQ ID NO: 23 - protein, 412 aa, synthetic construct
  REGION 1..412: note = exemplary polypeptide
MQYDVAIKNGNVFTGTEFIGKMNIYISNGKISKVSKEDLNAKQEIDAKDMLVMPGLIDAHMHISGLKGGSLLKIMFEKPEYRALRATKWLEKLLLAGFTTVRDCGEPISISLKRAVNEGYIKGPKIIAAGKPITQTFGHGEFSHDIPLEFSISLGFSEICDGTESCIHAARKVLRDGSDFIKIFATGGVLSQRDRPDMPQLSFDEIRAIVNEAEKAGTYVAAHAHGDKGARIAIEAGIKTLEHGSLLKEDTLKLMLEKNISLTPTLTITEIIYKYGKTIGVDEWGLEKIVEVRENLHKVLRKAKELGITIITGTHMGFETGLEDYDIGKNWTEILLLTEVGGLSNEEALRAATYNSAKALGLNSGLIDEGKDADIIVIDGNPLENIKDVSKVVHVIKQGKLEVLDSKIINNY

SEQ ID NO: 24 - protein, 408 aa, synthetic construct
  REGION 1..408: note = exemplary polypeptide
MELAIRNGIVFNGKEIIGKTNIYVREGKIVEVSREDHSAKMEIDAKDMFVMPGLIDAHIHLSGIKGGSLLKIMFEKPEYRVLRAAKWLEKLLLAGFTTVRDCGETVSLALKRALNDGIIGGPKVIAAGKPITQTFGHGELSHDVPLEFSKTMSFSEFCDGMESCIHAARKVLRDGADFIKIFATGGVLSQRDRPEHPQLSYEEIRAIVNEAEKVNTYVAAHAHGDRGARIAIEAGVKTLEHGTLLKDETLKLMKEKNVTLTPTLTIQELIFKYGKQIGVDEWGLQKITSVRENIANVVRKAKEYGVTIISGTDLGFETGLEEIDMGKNWMETVLLVERGGLSPIEALRASTYNAAMAIGINAGVIEIGKDADIIIINGDPTLNVREISKITHVVKNGKLTVENKSIKG

SEQ ID NO: 25 - protein, 411 aa, synthetic construct
  REGION 1..411: note = exemplary polypeptide
MFDLGIKNGNVFSGKDNLGKTNIYVNNGKIVKISKENLEVRQEIDAKDMFVMPGLIDSHIHLSGIKGGSLLKIMFEKPEYRILRASKWLEKLLLAGFTTVRDCGDPISISLAKGVKDGIIIGPNIIAAGRPITQTFGHGEISHDIPLEFSEVLGFSEICDGVESCIHSARKVLREGSDFIKIFATGGVLSQRDRPENPQLTYEEIKSIVNEAEKIGTYVAAHAHGDKGARIAIEAGIKTLEHGTMLKDDTMKLMIEKDVSLTPTLTIQEVIYKYGKQIGVDEWGLEKITEVRNSISSVVKKAKEYGIRIITGTDLGFETGLSDIDIGKNWVELTLLVERGGLTPKEALQAATYNSALSLRINSGLIDTDRNADIIIIDGDPEENISDIAKIDHVIKNGNIIVEKGKIINYS

SEQ ID NO: 26 - protein, 410 aa, synthetic construct
  REGION 1..410: note = exemplary polypeptide
MAFDIALKNGNIFNGKEFIGKSNIYINNGKIVKVSKEELDAKQEIDAMDMFVMPGLIDAHMHISGIKGGSLLKLMFERPEYRTIRATKWLEKLLLAGFTTVRDCGDPISISLAKAVKEGIIKGPNVIAAGRPITQTFGHGELSHDIPLEVSETTGFSEICDGTESCIHAARKVLREGSDFIKIFATGGVLSQRDKPENPQLTYDEIKVIVNEAEKVGTYVAAHAHGDKGARIAIEAGIKTLEHGTLLQDETLKLMIEKKVTLTPTLSIQEVIYRHGKEIGVDEWGLEKIAMVRDSISSVVKKAKDYGITIITGTDLGFETGLEDIDIGKNWMEIVLLIEIGGLTPVEALRAATYNSSIAININSGLIDSGKEADLIIINGNPLEDIKDISKVSYVIKDGKIEVEERKIKN

SEQ ID NO: 27 - protein, 409 aa, synthetic construct
  REGION 1..409: note = exemplary polypeptide
MDILIKGGQVFNGKDFIGKANVYIKYGKIVEVSKEVKEAKEEIDARDMFVMPGLIDAHIHLSGIKGGSLMKTMFERPEYRVLRASKWLEKLLLAGFTTVRDCGEYVSLALKRAVQEGTIKGSNIIAAGRPVTQTFGHGEISHDIPLEFSHKVGFSEFCDGTESCIHAARKILRDGSDFIKVFATGGVLSQRDKPENPQLSYDEIKAIVNEAHKVGTYVAAHAHGDAGAKIAVEAGVKTIEHGTLLKDDTLKLMVNKGVSLTPTLTIQEVIYRYGKQIGVDEWGLQKIFEVRERVADVVKKAKDYGVLMISGTDLGFETGLWDIDMGKNWYELVLLVERGGLTPKEALTTATYNSSIALGINSGFIDIGKEGDLVVIDGNPLENIYDIAKVTHVLKAGRVEVRNGKLESK

SEQ ID NO: 28 - protein, 410 aa, synthetic construct
  REGION 1..410: note = exemplary polypeptide
MDISIKGGELFTGKELLGKFNIYIKDKKIVEISKEDKQAEVEVNAKDMFVMPGLIDAHIHLSGVKGGSLLKTMFEKPEYRVLRASKWLERLLLAGFTTVRDCGESISIALKRAVNEKIIKGPKIIAAGRPISQTFGHGELSHDLPLEFSKALSFSEFCDGVESCIHASRKVLREGADFIKIFATGGVLSQRDKPENPQLSYEEIKAIVNEAEKVNTYVAAHAHGDKGARIAIEAGVKTLEHGTLLRDDTLKLMVEKRVSLTPTLTIQELIYKYGKQIGVDEWGLEKINSVRESVAEVVRKAKEYGVLIINGTDLGFETGLDEIDMGKNWMETVLLVERGNLSNVEALRASTYNSAIAIGNDAGIIDIGRPADIIVINGNPSENIRDISKVVHVIKDGSLVVENKRLTDSL

SEQ ID NO: 29 - protein, 407 aa, synthetic construct
  REGION 1..407: note = exemplary polypeptide
MDLGIKNGNVFNGKGVVGKVNIYVKDGKIVEISKENKQAKIEIDAKDMFVMPGLIDAHIHLTGIKGGSLLKTMFEKPEYRVLRASKWLEKLLLAGFTTVRDCGETISLALKRGVSEGIINGPRIIAAGKPISQTFGHGELSHDVPLEFSMALSFSEFCDGVESCIRASRKVLRDGADFIKIFATGGVLSQRDRPEHPQLSYEEISAIVNEAEKINTYVAAHAHGDKGARIAVEAGIKTLEHGTLLRDETLKLMRERNVTLTPTLTIQELIYKYGKQIGVDEWGLQKINEVRESVVNVVKKAEEYGVMIITGTDLGFETGLEEIDIGKNWMESVLLVERGGLSTISSLRASTYNSAFAIGINAGAIEVGKDADIIVIDGDPTENIRDLSKVRRVVKNGKLTVEDGKFL

SEQ ID NO: 30 - protein, 408 aa, synthetic construct
  REGION 1..408: note = exemplary polypeptide
MELAIRNGIVFNGKEVIGKTNIYVREGKIVEVSREDHSAKMEIDAKDMFVMPGLIDAHIHLSGIKGGSLLKIMFEKPEYRVLRAAKWLEKLLLAGFTTVRDCGETVSLALKRAVNDGIIDGPKVIAAGKPITQTFGHGELSHDVPLEFSKTMSFSEFCDGMESCIHAARKVLRDGADFIKIFATGGVLSQRDTPEHPQLSYEEIRAIVNEAEKVNSYVAAHAHGDRGARIAIEAGVKTLEHGTLLKDETLKLMKEKNVTLTPTLTIQELIFKYGKQIGVDEWGLQKITSVRESIANVVRKAKEYGVTIISGTDLGFETGLEEIEMGKNWMETVLLVERGGLSPIEALRASTYNAAMAIGINAGVIEIGKDADIIIINGDPTQNVREISKITHVIKNGKLTVENKSIKG

SEQ ID NO: 31 - protein, 408 aa, synthetic construct
  REGION 1..408: note = exemplary polypeptide
MELAIRNGIVFNGKEVIGKTNIYVREGKIVEVSREDHSAKMEIDAKDMFVMPGLIDAHIHLSGIKGGSLLKIMFEKPEYRVLRAAKWLEKLLLAGFTTVRDCGETVSLALKRAVNDGIVDGPKVIAAGKPITQTFGHGELSHDVPLEFSKTMSFSEFCDGMESCIHAARKVLRDGADFIKIFATGGVLSQRDTPEHPQLSYEEIRAIVNEAEKVNSYVAAHAHGDRGARIAIEAGVKTLEHGTLLKDETLKLMKEKNVTLTPTLTIQELIFKYGKQIGVDEWGLQKITSVRESIANVVRKAKEYGVTIISGTDLGFETGLEEIEMGKNWMETVLLVERGGLSPIEALRASTYNAAMAIGINAGVIEIGKDADIIIINGDPTQNVREISKITHVIKNGKLTVENKSIKG

SEQ ID NO: 32 - protein, 408 aa, synthetic construct
  REGION 1..408: note = exemplary polypeptide
MELAIRNGIVFNGKEVIGKTNIYVREEKIVEISREDHSAKMEIDAKDMFVMPGLIDAHIHLSGIKGGSLLKIMFEKPEYRVLRAAKWLEKLLLAGFTTVRDCGETVSLALKRALNDGIIDGPKVIAAGKPITQTFGHGELSHDVPLEFSKTMSFSEFCDGMESCIHAARKVLRDGADFIKIFATGGVLSQRDRPEHPQLSYEEIRAIVNEAEKVNTYVAAHAHGDRGARIAIEAGVKTLEHGTLLKDETLKLMKEKNVTLTPTLTIQELIFKYGKQIGVDEWGLQKITSVRENIANVVRKAKEYGVTIISGTDLGFETGLEEIDMGKNWMETVLLVERGGLSPIEALRASTYNAAMAIGINAGVIEIGKDADIIIINGDPTLNVREISKITHVIKNGKLTVENKSIKG

SEQ ID NO: 33 - protein, 408 aa, synthetic construct
  REGION 1..408: note = exemplary polypeptide
MELAIRNGIVFNGKEIIGKTNIYVREGKIVEVSREDHSAKMEIDAKDMFVMPGLIDAHIHLSGIKGGSLLKIMFEKPEYRVLSAAKWLEKLLLAGFTTVRDCGETVSLALKRALNDGIIGGPKVIAAGKPITQTFGHGELSHDVPLEFSKTMSFSEFCDGMESCIHAARKVLRDGADFIKIFATGGVLSQRDRPEHPQLSYEEIRAIVNEAEKVNTYVAAHAHGDRGARIAIEAGVKTLEHGTLLKDETLKLMKEKNVTLTPTLTIQELIFKYGKQIGVDEWGLQKITSVRENIANVVRKAKEYGVTIISGTDLGFETGLEEIDMGKNWMETVLLVERGGLSPIEALRASTYNAAMAIGINAGVIEIGKDADIIIINGDPTLNVREISKITHVVKNGKLTVENKSIKG

SEQ ID NO: 34 - protein, 408 aa, synthetic construct
  REGION 1..408: note = exemplary polypeptide
MELAIRNGIVFNGKEIIGKTNIYVREGKIVEVSREDHSAKMEIDAKDMFVMPGLIDAHIHLSGIKGGSLLKIMFEKPEYRVLRAAKWLEKLLLAGFTTVRDCGETVSLALKRALNDGIIGGPKVIAAGKPITQTFGHGELSHDVPLEFSKTMSFSEFCDGMESCIHAARKVLRDGADFIKIFATGGVLSQRDRPEHPQLSYEEIRAIVNEAEKVNTYVAAHAHGDRGARIAIEAGVKTLEHGTLLKDETLKLMKEKNVTLTPTLTIQELIFKYGKQIGVDEWGLQKITSVRENIANVVRKAKEYGVTIISGTDLGFETGLEEIDMGKNWMETVLLVERGGLSPIEALRASTYNAAMAIGINAGVIEIGKDADIIIINGDPTLNVREISKITHVIKNGKLTVENKSIKG

SEQ ID NO: 35 - protein, 408 aa, synthetic construct
  REGION 1..408: note = exemplary polypeptide
MYSLAIRNGIVFDGEKFIGKTNIYVDGDKIVKITNERLDAKEEVNAEGYFVMPGLIDAHLHLTGGRGGSLLMGYLLDDPRTRLLRAANWARELLMAGFTTVRDCGEPNSIILRNAINEGIIPGPRIIAAGSPITQTFGHAELSHSLPLEWSIQLGFGEICDGVDECIKAARKVLRSGADFIKIMATGGVLSERDRPEWPQLTPEEMSAIVREAEKVGTYVAAHAHGDKGARIAVEAGVRTIEHGSLITEETVKLMAEKGVSLTPTLSIQELIYKYGKQMGIDPWGLEKIGQVREGVINSIKLARKYGVMILAGTDLGFKTGLDIDMGKNWVEVKLLVELGGLSSEEALRAATGNASKMLRLNTGIIREGYLADIVLINGNPLENINDIARVSTVIKGGKVTVENGKLI

SEQ ID NO: 36 - protein, 408 aa, synthetic construct
  REGION 1..408: note = exemplary polypeptide
MYSLAIRNGIVFDGEKFIGKTNIYVDGDKIVKITNERLDAKEEVNAEGYFVMPGLIDAHLHLTGARGGSLLMGYLLDDPRTRLLRAANWARELLMAGFTTVRDCGEPNSIILRNAINEGIIPGPRIIAAGSPITQTFGHAELSHSLPLEWSIQLGFGEICDGVDECIKAARKVLRSGADFIKIMATGGVLSERDRPEWPQLTPEEMSAIVREAEKVGTYVAAHAHGDKGARIAVEAGVRTIEHGSLITEETVKLMAEKGVSLTPTLSIQELIYKYGKQMGIDPWGLEKIGQVREGVINSIKLARKYGVMILAGTDLGFKTGLDIDMGKNWVEVKLLVELGGLSSEEALRAATGNASKMLRLNTGIIREGYLADIVLINGNPLENINDIARVSTVIKGGKVTVENGKLI

SEQ ID NO: 37 - protein, 408 aa, synthetic construct
  REGION 1..408: note = exemplary polypeptide
MYSLAIRNGIVFDGEKFIGKTNIYVDGDKIVKITNERLDAKEEVNAEGYFVMPGLIDAHLHLTGIRGGSLLMGYLLDDPRTRLLRAANWARELLMAGFTTVRDCGGPNSIILRNAINEGIIPGPRIIAAGSPITQTFGHAELSHSLPLEWSIQLGFGEICDGVDECIKAARKVLRSGADFIKIMATGGVLSERDRPEWPQLTPEEMSAIVREAEKVGTYVAAHAHGDKGARIAVEAGVRTIEHGSLITEETVKLMAEKGVSLTPTLSIQELIYKYGKQMGIDPWGLEKIGQVREGVINSIKLARKYGVMILAGTDLGFKTGLDIDMGKNWVEVKLLVELGGLSSEEALRAATGNASKMLRLNTGIIREGYLADIVLINGNPLENINDIARVSTVIKGGKVTVENGKLI

SEQ ID NO: 38 - protein, 408 aa, synthetic construct
  REGION 1..408: note = exemplary polypeptide
MYSLAIRNGIVFDGEKFIGKTNIYVDGDKIVKITNERLDAKEEVNAEGYFVMPGLIDAHLHLTGIRGGSLLMGYLLDDPRTRLLRAANWARELLMAGFTTVRDCGSPNSIILRNAINEGIIPGPRIIAAGSPITQTFGHAELSHSLPLEWSIQLGFGEICDGVDECIKAARKVLRSGADFIKIMATGGVLSERDRPEWPQLTPEEMSAIVREAEKVGTYVAAHAHGDKGARIAVEAGVRTIEHGSLITEETVKLMAEKGVSLTPTLSIQELIYKYGKQMGIDPWGLEKIGQVREGVINSIKLARKYGVMILAGTDLGFKTGLDIDMGKNWVEVKLLVELGGLSSEEALRAATGNASKMLRLNTGIIREGYLADIVLINGNPLENINDIARVSTVIKGGKVTVENGKLI

SEQ ID NO: 39 - protein, 408 aa, synthetic construct
  REGION 1..408: note = exemplary polypeptide
MYSLAIRNGIVFDGEKFIGKTNIYVDGDKIVKITNERLDAKEEVNAEGYFVMPGLIDAHLHLTGIRGGSLLMGYLLDDPRTRLLRAANWARELLMAGFTTVRDCGEPNSIILRNAINEGIIPGPRIIAAGSPITQTFGHAELSHSLPLEWSIQLGFGEICDGVDECIKAARKVLRSGADFIKIMATGGVLSERDRPEWPQLTPEEMSAIVREAEKVGTYVAAHAHGDKGARIAVEAGVRTIEHGSLITEETVKLMAEKGVSLTPTLSAQELIYKYGKQMGIDPWGLEKIGQVREGVINSIKLARKYGVMILAGTDLGFKTGLDIDMGKNWVEVKLLVELGGLSSEEALRAATGNASKMLRLNTGIIREGYLADIVLINGNPLENINDIARVSTVIKGGKVTVENGKLI

SEQ ID NO: 40 - protein, 408 aa, synthetic construct
  REGION 1..408: note = exemplary polypeptide
MYSLAIRNGIVFDGEKFIGKTNIYVDGDKIVKITNERLDAKEEVNAEGYFVMPGLIDAHLHLTGIRGGSLLMGYLLDDPRTRLLRAANWARELLMAGFTTVRDCGEPNSIILRNAINEGIIPGPRIIAAGSPITQTFGHAELSHSLPLEWSIQLGFGEICDGVDECIKAARKVLRSGADFIKIMATGGVLSERDRPEWPQLTPEEMSAIVREAEKVGTYVAAHAHGDKGARIAVEAGVRTIEHGSLITEETVKLMAEKGVSLTPTLSIIELIYKYGKQMGIDPWGLEKIGQVREGVINSIKLARKYGVMILAGTDLGFKTGLDIDMGKNWVEVKLLVELGGLSSEEALRAATGNASKMLRLNTGIIREGYLADIVLINGNPLENINDIARVSTVIKGGKVTVENGKLI

SEQ ID NO: 41 - protein, 408 aa, synthetic construct
  REGION 1..408: note = exemplary polypeptide
MYSLAIRNGIVFDGEKFIGKTNIYVDGDKIVKITNERLDAKEEVNAEGYFVMPGLIDAHLHLTGIRGGSLLMGYLLDDPRTRLLRAANWARELLMAGFTTVRDCGEPNSIILRNAINEGIIPGPRIIAAGSPITQTFGHAELSHSLPLEWSIQLGFGEICDGVDECIKAARKVLRSGADFIKIMATGGVLSERDRPEWPQLTPEEMSAIVREAEKVGTYVAAHAHGDKGARIAVEAGVRTIEHGSLITEETVKLMAEKGVSLTPTLSIQELIYKVGKQMGIDPWGLEKIGQVREGVINSIKLARKYGVMILAGTDLGFKTGLDIDMGKNWVEVKLLVELGGLSSEEALRAATGNASKMLRLNTGIIREGYLADIVLINGNPLENINDIARVSTVIKGGKVTVENGKLI

SEQ ID NO: 42 - protein, 408 aa, synthetic construct
  REGION 1..408: note = exemplary polypeptide
MYSLAIRNGIVFDGEKFIGKTNIYVDGDKIVKITNERLDAKEEVNAEGYFVMPGLIDAHLHLTGIRGGSLLMGYLLDDPRTRLLRAANWARELLMAGFTTVRDCGEPNSIILRNAINEGIIPGPRIIAAGSPITQTFGHAELSHSLPLEWSIQLGFGEICDGVDECIKAARKVLRSGADFIKIMATGGVLSERDRPEWPQLTPEEMSAIVREAEKVGTYVAAHAHGDKGARIAVEAGVRTIEHGSLITEETVKLMAEKGVSLTPTLSIQELIYKYGKQMGIDPWGLEKIGQVREGVINSIKLARKYGVMILAGTDLGVKTGLDIDMGKNWVEVKLLVELGGLSSEEALRAATGNASKMLRLNTGIIREGYLADIVLINGNPLENINDIARVSTVIKGGKVTVENGKLI

SEQ ID NO: 43 - protein, 408 aa, synthetic construct
  REGION 1..408: note = exemplary polypeptide
MYSLAIRNGIVFDGEKFIGKTNIYVDGDKIVKITNERLDAKEEVNAEGYFVMPGLIDAHLHLTGIRGGSLLMGYLLDDPRTRLLRAANWARELLMAGFTTVRDCGEPNSIILRNAINEGIIPGPRIIAAGSAITQTFGHAELSHSLPLEWSIQLGFGEICDGVDECIKAARKVLRSGADFIKIMATGGVLSERDRPEWPQLTPEEMSAIVREAEKVGTYVAAHAHGDKGARIAVEAGVRTIEHGSLITEETVKLMAEKGVSLTPTLSIQELIYKYGKQMGIDPWGLEKIGQVREGVINSIKLARKYGVMILAGTDLGFKTGLDIDMGKNWVEVKLLVELGGLSSEEALRAATGNASKMLRLNTGIIREGYLADIVLINGNPLENINDIARVSTVIKGGKVTVENGKLI

SEQ ID NO: 44 - protein, 408 aa, synthetic construct
  REGION 1..408: note = exemplary polypeptide
MYSLAIRNGIVFDGEKFIGKTNIYVDGDKIVKITNERLDAKEEVNAEGYFVMPGLIDAHLHLTGIRGGSLLMGYLLDDPRTRLLRAANWARELLMAGFTTVRDCGEPNSIILRNAINEGIIPGPRIIAAGSPITQTFGHAELSHSLPLEWSIQLGFGEICDGVDECIKAARKVLRSGADFIKIMATGGVLSERDRPEWPQLTPEEMSAIVREAEKVGTYVAAHAHGDKGARIAVEAGVRTIEHGSLITEETVKLMAEKGVSLTPTLSIQELVYKYGKQMGIDPWGLEKIGQVREGVINSIKLARKYGVMILAGTDLGFKTGLDIDMGKNWVEVKLLVELGGLSSEEALRAATGNASKMLRLNTGIIREGYLADIVLINGNPLENINDIARVSTVIKGGKVTVENGKLI

SEQ ID NO: 45 - protein, 408 aa, synthetic construct
  REGION 1..408: note = exemplary polypeptide
MYSLAIRNGIVFDGEKFIGKTNIYVDGDKIVKITNERLDAKEEVNAEGYFVMPGLIDAHLHLTGIRGGSLLMGYLLDDPRTRLLRAANWARELLMAGFTTVRDCGEPNSIILRNAINEGIIPGPRIIAAGSPITQTFGHAELSHSLPLEWSIQLGFGEICDGVDECIKAARKVLRSGADFIKIMATGGVLSERDRPEWPQLTPEEMSAIVREAEKVGTYVAAHAHGDKGARIAVEAGVRTIEHGSLITEETVKLMAEKGVSLTPTLSIQELIYKFGKQMGIDPWGLEKIGQVREGVINSIKLARKYGVMILAGTDLGFKTGLDIDMGKNWVEVKLLVELGGLSSEEALRAATGNASKMLRLNTGIIREGYLADIVLINGNPLENINDIARVSTVIKGGKVTVENGKLI

SEQ ID NO: 46 - protein, 408 aa, synthetic construct
  REGION 1..408: note = exemplary polypeptide
MYSLAIRNGIVFDGEKFIGKTNIYVDGDKIVKITNERLDAKEEVNAEGYFVMPGLIDAHLHLTGIRGGSLLMGYLLDDPRTRLLRAANWARELLMAGFTTVRDCGEPNSIILRNAINEGIIPGPRIIAAGSPITQTFGHAELSHSLPLEWSIQLGFGEICDGVDECIKAARKVLRSGADFIKIMATGGVLSERDRPEWPQLTPEEMSAIVREAEKVGTYVAAHAHGDKGARIAVEAGVRTIEHGSLITEETVKLMAEKGVSLTPTLSIQELIYKHGKQMGIDPWGLEKIGQVREGVINSIKLARKYGVMILAGTDLGFKTGLDIDMGKNWVEVKLLVELGGLSSEEALRAATGNASKMLRLNTGIIREGYLADIVLINGNPLENINDIARVSTVIKGGKVTVENGKLI

SEQ ID NO: 47 - protein, 408 aa, synthetic construct
  REGION 1..408: note = exemplary polypeptide
MYSLAIRNGIVFDGEKFIGKTNIYVDGDKIVKITNERLDAKEEVNAEGYFVMPGLIDAHLHLTGARGGSLLMGYLLDDPRTRLLRAANWARELLMAGFTTVRDCGGPNSIILRNAINEGIIPGPRIIAAGSAITQTFGHAELSHSLPLEWSIQLGFGEICDGVDECIKAARKVLRSGADFIKIMATGGVLSERDRPEWPQLTPEEMSAIVREAEKVGTYVAAHAHGDKGARIAVEAGVRTIEHGSLITEETVKLMAEKGVSLTPTLSAIELVYKVGKQMGIDPWGLEKIGQVREGVINSIKLARKYGVMILAGTDLGVKTGLDIDMGKNWVEVKLLVELGGLSSEEALRAATGNASKMLRLNTGIIREGYLADIVLINGNPLENINDIARVSTVIKGGKVTVENGKLI

SEQ ID NO: 48 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFADNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 49 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSADWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 50 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYPVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 51 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYGVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 52 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDGIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 53 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDSIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 54 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGALSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 55 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVVSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 56 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVMSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 57 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISSIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 58 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISGIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 59 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAVAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 60 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHTEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 61 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGTGAHGSNAREFRYMVENGLTPAQALLAGTANAARLLGTFDRVGTLEAGKLADVVAVPGNPIADITVTERPLFVMKDGVVVRRDAVR

SEQ ID NO: 62 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPPDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 63 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPQLSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 64 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGETDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 65 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSWAREFRYMVENGLTPAQALLAGTANAARLLGTFDRVGTLEAGKLADVVAVPGNPIADITVTERPLFVMKDGVVVRRDAVR

SEQ ID NO: 66 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHAEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 67 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGAGAHGSNAREFRYMVENGLTPAQALLAGTANAARLLGTFDRVGTLEAGKLADVVAVPGNPIADITVTERPLFVMKDGVVVRRDAVR

SEQ ID NO: 68 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGPHGSNAREFRYMVENGLTPAQALLAGTANAARLLGTFDRVGTLEAGKLADVVAVPGNPIADITVTERPLFVMKDGVVVRRDAVR

SEQ ID NO: 69 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADMDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 70 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADLDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 71 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAQGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 72 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFCPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 73 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFAPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 74 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYLVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 75 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGQADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 76 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGLADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 77 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISGIAHAEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 78 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISGIAHTEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 79 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISGIAHVEEKIGTFPPVIAEKGRAAAAHLRQTFRHAVQIGVPIALGTDAGTGAHGSNAREFRYMVENGLTPAQALLAGTANAARLLGTFDRVGTLEAGKLADVVAVPGNPIADITVTERPLFVMKDGVVVRRDAVR

SEQ ID NO: 80 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISGIAHVEEKIGTFPPVIAEKGRAAAAHLRQTFRHAVQIGVPIALGTDAGAGAHGSNAREFRYMVENGLTPAQALLAGTANAARLLGTFDRVGTLEAGKLADVVAVPGNPIADITVTERPLFVMKDGVVVRRDAVR

SEQ ID NO: 81 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGLLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 82 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGILSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 83 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVISLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 84 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHANGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 85 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHALGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 86 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISTIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 87 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAAAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 88 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHLEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 89 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHIEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 90 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAAVGAHGSNAREFRYMVENGLTPAQALLAGTANAARLLGTFDRVGTLEAGKLADVVAVPGNPIADITVTERPLFVMKDGVVVRRDAVR

SEQ ID NO: 91 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTLPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 92 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTMPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 93 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAGAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 94 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAVAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 95 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGLGAHGSNAREFRYMVENGLTPAQALLAGTANAARLLGTFDRVGTLEAGKLADVVAVPGNPIADITVTERPLFVMKDGVVVRRDAVR

SEQ ID NO: 96 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGIGAHGSNAREFRYMVENGLTPAQALLAGTANAARLLGTFDRVGTLEAGKLADVVAVPGNPIADITVTERPLFVMKDGVVVRRDAVR

SEQ ID NO: 97 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDVFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 98 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDLFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 99 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFVDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 100 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFLDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 101 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNIMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 102 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNWMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 103 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPVPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 104 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPFPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 105 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPQKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 106 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPNKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 107 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAQVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 108 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIANVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 109 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEQIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 110 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEEIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 111 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAQLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 112 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAVLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 113 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIQDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 114 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAQGSNAREFRYMVENGLTPAQALLAGTANAARLLGTFDRVGTLEAGKLADVVAVPGNPIADITVTERPLFVMKDGVVVRRDAVR

SEQ ID NO: 115 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAYGSNAREFRYMVENGLTPAQALLAGTANAARLLGTFDRVGTLEAGKLADVVAVPGNPIADITVTERPLFVMKDGVVVRRDAVR

SEQ ID NO: 116 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGDSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 117 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDYFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 118 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMQRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 119 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALRGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 120 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYWARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 121 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFYDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 122 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSPDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 123 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKPGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 124 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGYFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 125 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTYPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 126 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESEAWGRKVAAHAHGDKAAKMAVAAGVRSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 127 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRYVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLRTFDRVGTLEAGKLADVVAVPGNPIADITVTERPLFVMKDGVVVRRDAVR

SEQ ID NO: 128 - protein, 440 aa, synthetic construct
  REGION 1..440: note = exemplary polypeptide
MTTRTRILTAFLSLLAANACAADAAPAAPHTVVLRCARMFDGASGKLVDHPQLTIVGNKIVRIAQGDKAEVPAGAEVVDLGDATCLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 129 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQCSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGCFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 130 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHCDQDPIPPHKLPLAGPLQGVCNGPEECRAAVRYQIKYGANVIKFCPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 131 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVCLVPTISAIAHVEEKIGTFPPVIAEKGRAAAAHLRQTFRHAVQIGVPIALGTDAGVGAHGSNAREFRYMVENGLTPAQALLAGTANAARCLGTFDRVGTLEAGKLADVVAVPGNPIADITVTERPLFVMKDGVVVRRDAVR

SEQ ID NO: 132 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAICHVEEKIGTFPPVIAEKGRAACAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 133 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISVIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 134 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDDMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 135 - protein, 440 aa, synthetic construct; identical to SEQ ID NO: 131
  REGION 1..440: note = exemplary polypeptide

SEQ ID NO: 136 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLDGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 137 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLNGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 138 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLPGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 139 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLTGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 140 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLVGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 141 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVASTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 142 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVPSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 143 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSDDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 144 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSEDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 145 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSPDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 146 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSRDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 147 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHPDQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 148 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHSDQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 149 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHTDQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 150 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYPVGSTGGHADQDPVPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 151 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYPVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAAAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 152 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYPVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAVAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 153 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYPVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTYPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 154 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYPVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAAVGAHGSNAREFRYMVENGLTPAQALLAGTANAARLLGTFDRVGTLEAGKLADVVAVPGNPIADITVTERPLFVMKDGVVVRRDAVR

SEQ ID NO: 155 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPVPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAAAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 156 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPVPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAVAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 157 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPVPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTYPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 158 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPVPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAAVGAHGSNAREFRYMVENGLTPAQALLAGTANAARLLGTFDRVGTLEAGKLADVVAVPGNPIADITVTERPLFVMKDGVVVRRDAVR

SEQ ID NO: 159 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAAAHVEEKIGTYPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 160 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAVAHVEEKIGTYPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 161 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAAAHVEEKIGTFPPVIAEKGRAAAAHLRQTFRHAVQIGVPIALGTDAAVGAHGSNAREFRYMVENGLTPAQALLAGTANAARLLGTFDRVGTLEAGKLADVVAVPGNPIADITVTERPLFVMKDGVVVRRDAVR

SEQ ID NO: 162 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAVAHVEEKIGTFPPVIAEKGRAAAAHLRQTFRHAVQIGVPIALGTDAAVGAHGSNAREFRYMVENGLTPAQALLAGTANAARLLGTFDRVGTLEAGKLADVVAVPGNPIADITVTERPLFVMKDGVVVRRDAVR

SEQ ID NO: 163 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTYPPVIAEKGRAAAAHLRQTFRHAVQIGVPIALGTDAAVGAHGSNAREFRYMVENGLTPAQALLAGTANAARLLGTFDRVGTLEAGKLADVVAVPGNPIADITVTERPLFVMKDGVVVRRDAVR

SEQ ID NO: 164 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYPVGSTGGHADQDPVPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAAAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 165 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYPVGSTGGHADQDPVPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAVAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 166 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYPVGSTGGHADQDPVPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTYPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 167 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYPVGSTGGHADQDPVPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAAVGAHGSNAREFRYMVENGLTPAQALLAGTANAARLLGTFDRVGTLEAGKLADVVAVPGNPIADITVTERPLFVMKDGVVVRRDAVR

SEQ ID NO: 168 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYPVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAAAHVEEKIGTYPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 169 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYPVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAVAHVEEKIGTYPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 170 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYPVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAAAHVEEKIGTFPPVIAEKGRAAAAHLRQTFRHAVQIGVPIALGTDAAVGAHGSNAREFRYMVENGLTPAQALLAGTANAARLLGTFDRVGTLEAGKLADVVAVPGNPIADITVTERPLFVMKDGVVVRRDAVR

SEQ ID NO: 171 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYPVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAVAHVEEKIGTFPPVIAEKGRAAAAHLRQTFRHAVQIGVPIALGTDAAVGAHGSNAREFRYMVENGLTPAQALLAGTANAARLLGTFDRVGTLEAGKLADVVAVPGNPIADITVTERPLFVMKDGVVVRRDAVR

SEQ ID NO: 172 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYPVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTYPPVIAEKGRAAAAHLRQTFRHAVQIGVPIALGTDAAVGAHGSNAREFRYMVENGLTPAQALLAGTANAARLLGTFDRVGTLEAGKLADVVAVPGNPIADITVTERPLFVMKDGVVVRRDAVR

SEQ ID NO: 173 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPVPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAAAHVEEKIGTYPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 174 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPVPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAVAHVEEKIGTYPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 175 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPVPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAAAHVEEKIGTFPPVIAEKGRAAAAHLRQTFRHAVQIGVPIALGTDAAVGAHGSNAREFRYMVENGLTPAQALLAGTANAARLLGTFDRVGTLEAGKLADVVAVPGNPIADITVTERPLFVMKDGVVVRRDAVR

SEQ ID NO: 176 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPVPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAVAHVEEKIGTFPPVIAEKGRAAAAHLRQTFRHAVQIGVPIALGTDAAVGAHGSNAREFRYMVENGLTPAQALLAGTANAARLLGTFDRVGTLEAGKLADVVAVPGNPIADITVTERPLFVMKDGVVVRRDAVR

SEQ ID NO: 177 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPVPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTYPPVIAEKGRAAAAHLRQTFRHAVQIGVPIALGTDAAVGAHGSNAREFRYMVENGLTPAQALLAGTANAARLLGTFDRVGTLEAGKLADVVAVPGNPIADITVTERPLFVMKDGVVVRRDAVR

SEQ ID NO: 178 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAAAHVEEKIGTYPPVIAEKGRAAAAHLRQTFRHAVQIGVPIALGTDAAVGAHGSNAREFRYMVENGLTPAQALLAGTANAARLLGTFDRVGTLEAGKLADVVAVPGNPIADITVTERPLFVMKDGVVVRRDAVR

SEQ ID NO: 179 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAVAHVEEKIGTYPPVIAEKGRAAAAHLRQTFRHAVQIGVPIALGTDAAVGAHGSNAREFRYMVENGLTPAQALLAGTANAARLLGTFDRVGTLEAGKLADVVAVPGNPIADITVTERPLFVMKDGVVVRRDAVR

SEQ ID NO: 180 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNEAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 181 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNFAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 182 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNRAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 183 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAALAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 184 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAMAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 185 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAVAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 186 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHIRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 187 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHMRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 188 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHVRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 189 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDALVGAHGSNAREFRYMVENGLTPAQALLAGTANAARLLGTFDRVGTLEAGKLADVVAVPGNPIADITVTERPLFVMKDGVVVRRDAVR

SEQ ID NO: 190 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDASVGAHGSNAREFRYMVENGLTPAQALLAGTANAARLLGTFDRVGTLEAGKLADVVAVPGNPIADITVTERPLFVMKDGVVVRRDAVR

SEQ ID NO: 191 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDATVGAHGSNAREFRYMVENGLTPAQALLAGTANAARLLGTFDRVGTLEAGKLADVVAVPGNPIADITVTERPLFVMKDGVVVRRDAVR

SEQ ID NO: 192 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAVVGAHGSNAREFRYMVENGLTPAQALLAGTANAARLLGTFDRVGTLEAGKLADVVAVPGNPIADITVTERPLFVMKDGVVVRRDAVR

SEQ ID NO: 193 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVAAHGSNAREFRYMVENGLTPAQALLAGTANAARLLGTFDRVGTLEAGKLADVVAVPGNPIADITVTERPLFVMKDGVVVRRDAVR

SEQ ID NO: 194 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVFAHGSNAREFRYMVENGLTPAQALLAGTANAARLLGTFDRVGTLEAGKLADVVAVPGNPIADITVTERPLFVMKDGVVVRRDAVR

SEQ ID NO: 195 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVPAHGSNAREFRYMVENGLTPAQALLAGTANAARLLGTFDRVGTLEAGKLADVVAVPGNPIADITVTERPLFVMKDGVVVRRDAVR

SEQ ID NO: 196 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVVAHGSNAREFRYMVENGLTPAQALLAGTANAARLLGTFDRVGTLEAGKLADVVAVPGNPIADITVTERPLFVMKDGVVVRRDAVR

SEQ ID NO: 197 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVYAHGSNAREFRYMVENGLTPAQALLAGTANAARLLGTFDRVGTLEAGKLADVVAVPGNPIADITVTERPLFVMKDGVVVRRDAVR

SEQ ID NO: 198 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNFYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 199 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNLYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 200 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNPYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 201 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNYYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 202 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVLAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 203 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVMAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 204 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVQAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 205 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVSAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 206 - protein, 440 aa, synthetic construct
  REGION 1..440: note = exemplary polypeptide
MTTRTRILTAFLSLLAANACAADAAPAAPETVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 207 - protein, 440 aa, synthetic construct
  REGION 1..440: note = exemplary polypeptide
MTTRTRILTAFLSLLAANACAADAAPAAPKTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 208 - protein, 440 aa, synthetic construct
  REGION 1..440: note = exemplary polypeptide
MTTRTRILTAFLSLLAANACAADAAPAAPPTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 209 - protein, 440 aa, synthetic construct
  REGION 1..440: note = exemplary polypeptide
MTTRTRILTAFLSLLAANACAADAAPAAPRTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 210 - protein, 440 aa, synthetic construct
  REGION 1..440: note = exemplary polypeptide
MTTRTRILTAFLSLLAANACAADAAPAAPHTVVLRAARMFDGASGKLVDDPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 211 - protein, 440 aa, synthetic construct
  REGION 1..440: note = exemplary polypeptide
MTTRTRILTAFLSLLAANACAADAAPAAPHTVVLRAARMFDGASGKLVDNPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 212 - protein, 440 aa, synthetic construct
  REGION 1..440: note = exemplary polypeptide
MTTRTRILTAFLSLLAANACAADAAPAAPHTVVLRAARMFDGASGKLVDPPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 213 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQDSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 214 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDDWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 215 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDLWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 216 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDQWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 217 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARIELEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 218 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPDKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 219 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPEKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 220 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPGKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 221 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPPKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 222 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHDLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 223 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHELPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 224 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHRLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 225 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSAGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 226 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSPGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 227 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSSGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 228 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLDLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 229 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLPLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 230 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLTLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 231 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSNPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 232 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSQPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 233 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSSPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 234 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESAAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 235 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESNAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 236 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGDFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 237 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGHFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 238 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGTFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 239 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIADVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 240 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAEVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 241 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAELRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 242 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAARLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 243 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAASLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 244 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFREAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 245 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRKAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 246 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRNAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 247 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRSAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 248 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDSGVGAHGSNAREFRYMVENGLTPAQALLAGTANAARLLGTFDRVGTLEAGKLADVVAVPGNPIADITVTERPLFVMKDGVVVRRDAVR

SEQ ID NO: 249 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAAGSNAREFRYMVENGLTPAQALLAGTANAARLLGTFDRVGTLEAGKLADVVAVPGNPIADITVTERPLFVMKDGVVVRRDAVR

SEQ ID NO: 250 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAKGSNAREFRYMVENGLTPAQALLAGTANAARLLGTFDRVGTLEAGKLADVVAVPGNPIADITVTERPLFVMKDGVVVRRDAVR

SEQ ID NO: 251 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGANGSNAREFRYMVENGLTPAQALLAGTANAARLLGTFDRVGTLEAGKLADVVAVPGNPIADITVTERPLFVMKDGVVVRRDAVR

SEQ ID NO: 252 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSEAREFRYMVENGLTPAQALLAGTANAARLLGTFDRVGTLEAGKLADVVAVPGNPIADITVTERPLFVMKDGVVVRRDAVR

SEQ ID NO: 253 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSQAREFRYMVENGLTPAQALLAGTANAARLLGTFDRVGTLEAGKLADVVAVPGNPIADITVTERPLFVMKDGVVVRRDAVR

SEQ ID NO: 254 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFKNMMRFPAEQALYGAYYARITLEAGFTSVRDVGSTDWVALGLRNAIDAGKVPGPRMLVSNYAVGSTGGHADQDPIPPHDLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 255 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFKNMMRFPAEQALYGAYYARITLEAGFTSVRDVGSTDWVALGLRNAIDAGKVPGPRMLVSNYAVGSTGGHADQDPIPPHELPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 256 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPNRLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 257 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGDFIEDDTLRAMKAKNVYLVPTISAIAHVEEKIGTFPPVIAEKGRASAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 258 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGDFIEDDTLRAMKAKNVYLVPTISAIAHVEEKIGTFPPVIAEKGRATAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 259 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIADVEAKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 260 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAEVEAKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 261 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSSQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 262 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSVQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 263 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWNGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 264 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWRGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 265 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYPDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 266 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGRFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 267 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGYFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 268 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNFMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 269 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNKMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 270 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMDRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 271 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMRRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 272 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEEALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 273 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEHALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 274 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEKALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 275 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEMALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 276 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTSGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 277 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLLGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 278 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLMGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 279 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLVGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 280 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGTCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 281 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSFSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 282 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPADAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 283 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPDDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 284 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPFDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 285 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPLDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 286 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSESDPVDAPELSQDEMNAVIGESHAWGRKVAAHAHGDKAAKMAVAAGVDSIEHGSFIEDDTLRAMKAKNVYLVPTISAIAHVEEKIGTFPPTIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 287 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVISLSDPVDAPELSQDEMNAVIGESHAWGRKVAAHAHGDKAAKMAVAAGVDSIEHGSFIEDDTLRAMKAKNVYLVPTISAAAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 288 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAAAHVEEKIGTFPPVIAEKGRAVAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 289 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAAAHVEEKIGTFPPVIAEKGRAAAAHLRQTFRHAVQIGVPIALGTDAGVFAHGSNAREFRYMVENGLTPAQALLAGTANAARLLGTFDRVGTLEAGKLADVVAVPGNPIADITVTERPLFVMKDGVVVRRDAVR

SEQ ID NO: 290 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAAAHVEEKIGTFPPVIAEKGRAAAAHLRQTFRHAVQIGVPIALGTDAGVPAHGSNAREFRYMVENGLTPAQALLAGTANAARLLGTFDRVGTLEAGKLADVVAVPGNPIADITVTERPLFVMKDGVVVRRDAVR

SEQ ID NO: 291 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAAAHVEEKIGTFPPVIAEKGRAAAAHLRQTFRHAVQIGVPIALGTDAGVYAHGSNAREFRYMVENGLTPAQALLAGTANAARLLGTFDRVGTLEAGKLADVVAVPGNPIADITVTERPLFVMKDGVVVRRDAVR

SEQ ID NO: 292 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVISLSDPVDAPELSQDEMNAVIGESHAWGRKVAAHAHGDKAAKMAVAAGVDSIEHGSFIEDDTLRAMKAKNVYLVPTISAVAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 293 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAVAHVEEKIGTFPPVIAEKGRAVAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 294 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAVAHVEEKIGTFPPVIAEKGRAAAAHLRQTFRHAVQIGVPIALGTDAGVFAHGSNAREFRYMVENGLTPAQALLAGTANAARLLGTFDRVGTLEAGKLADVVAVPGNPIADITVTERPLFVMKDGVVVRRDAVR

SEQ ID NO: 295 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAVAHVEEKIGTFPPVIAEKGRAAAAHLRQTFRHAVQIGVPIALGTDAGVPAHGSNAREFRYMVENGLTPAQALLAGTANAARLLGTFDRVGTLEAGKLADVVAVPGNPIADITVTERPLFVMKDGVVVRRDAVR

SEQ ID NO: 296 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAVAHVEEKIGTFPPVIAEKGRAAAAHLRQTFRHAVQIGVPIALGTDAGVYAHGSNAREFRYMVENGLTPAQALLAGTANAARLLGTFDRVGTLEAGKLADVVAVPGNPIADITVTERPLFVMKDGVVVRRDAVR

SEQ ID NO: 297 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAAVVAHGSNAREFRYMVENGLTPAQALLAGTANAARLLGTFDRVGTLEAGKLADVVAVPGNPIADITVTERPLFVMKDGVVVRRDAVR

SEQ ID NO: 298 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAAVYAHGSNAREFRYMVENGLTPAQALLAGTANAARLLGTFDRVGTLEAGKLADVVAVPGNPIADITVTERPLFVMKDGVVVRRDAVR

SEQ ID NO: 299 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIADVEEKIGTFPPVIAEKGRAAAAHLRQTFRHAVQIGVPIALGTDAGLGAHGSNAREFRYMVENGLTPAQALLAGTANAARLLGTFDRVGTLEAGKLADVVAVPGNPIADITVTERPLFVMKDGVVVRRDAVR

SEQ ID NO: 300 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAEVEEKIGTFPPVIAEKGRAAAAHLRQTFRHAVQIGVPIALGTDAGLGAHGSNAREFRYMVENGLTPAQALLAGTANAARLLGTFDRVGTLEAGKLADVVAVPGNPIADITVTERPLFVMKDGVVVRRDAVR

SEQ ID NO: 301 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHAEEKIGTFPPVIAEKGRAAAAHLRQTFRHAVQIGVPIALGTDAGLGAHGSNAREFRYMVENGLTPAQALLAGTANAARLLGTFDRVGTLEAGKLADVVAVPGNPIADITVTERPLFVMKDGVVVRRDAVR

SEQ ID NO: 302 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAPLGAHGSNAREFRYMVENGLTPAQALLAGTANAARLLGTFDRVGTLEAGKLADVVAVPGNPIADITVTERPLFVMKDGVVVRRDAVR

SEQ ID NO: 303 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGLPAHGSNAREFRYMVENGLTPAQALLAGTANAARLLGTFDRVGTLEAGKLADVVAVPGNPIADITVTERPLFVMKDGVVVRRDAVR

SEQ ID NO: 304 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPVPPHKLPLLGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 305 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPVPPHKLPLMGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 306 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAAAHVEEKIGTFPPVIAEKGRAALAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 307 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAAAHVEEKIGTFPPVIAEKGRAAMAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 308 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAAAHVEEKIGTFPPVIAEKGRAAVAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 309 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAAAHVEEKIGTFPPVIAEKGRAAAAHIRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 310 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAAAHVEEKIGTFPPVIAEKGRAAAAHMRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 311 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAVAHVEEKIGTFPPVIAEKGRAALAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 312 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAVAHVEEKIGTFPPVIAEKGRAAMAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 313 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAVAHVEEKIGTFPPVIAEKGRAAVAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 314 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAVAHVEEKIGTFPPVIAEKGRAAAAHIRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 315 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAVAHVEEKIGTFPPVIAEKGRAAAAHMRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 316 - protein, 440 aa, synthetic construct
  REGION 1..440: note = exemplary polypeptide
MTTRTRILTAFLSLLAANACAADAAPAAPETVVLRCARMFDGASGKLVDNPQLTIVGNKIVRIAQGDKAEVPAGAEVVDLGDATCLPGFIDAHVHLVVQDSDQYRPRAYKDWQRFPAEHALRGAYWARIELEAGFTSVRDVAERDWVALGLRNAIDAGKVPGPRMLVSNFPVGSTSGHCDMDSVPPNRLPLVGPLQGTCNGPEECRAAVRYQIKYGANVIKFCPSSGAITFSNPADAPQLSPDEMNAVIGESEAWGRYVAAHAQGDKAAKMAVAAGVRSIEHGTFIQDDTLRAMKAKNVCLVPTISGVCEAEAQIGCYPPVMAEKGRAGMASMRQTFREAVQIGVPIALGTDSALPANGSQAREFRYMVENGLTPAQALLAGTANAARCLGTFDRVGTLEAGKLADVVAVPGNPIADITVTERPLFVMKDGVVVRRDAVR

SEQ ID NO: 317 - protein, 419 aa, synthetic construct
  REGION 1..419: note = exemplary polypeptide
ADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 318 - protein, 440 aa, synthetic construct
  REGION 1..440: note = exemplary polypeptide
MTTRTRILTAFLSLLAANACAADAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 319 - protein, 441 aa, synthetic construct
  REGION 1..441: note = exemplary polypeptide
MMITLRKLPLAVAVAAGVMSAQAMAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 320 - protein, 437 aa, synthetic construct
  REGION 1..437: note = exemplary polypeptide
MKKTAIAIAVALAGFATVAQAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 321 - protein, 437 aa, synthetic construct
  REGION 1..437: note = exemplary polypeptide
MKQSTIALALLPLLFTPVTKAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 322 - protein, 434 aa, synthetic construct
  REGION 1..434: note = exemplary polypeptide
MRVLLFLLLSLFMLPAFSAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 323 - protein, 435 aa, synthetic construct
  REGION 1..435: note = exemplary polypeptide
MKKIWLALAGLVLAFSASAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 324 - protein, 442 aa, synthetic construct
  REGION 1..442: note = exemplary polypeptide
MKNRNRMIVNCVTASLMYYWSLPALAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 325 - protein, 438 aa, synthetic construct
  REGION 1..438: note = exemplary polypeptide
MKYLLPTAAAGLLLLAAQPAMAAPAAPHTVVLRAARMFDGASGKLVDHPQLTIVGNKIVRIAQGDKAEVPAGAEVVDLGDATLLPGFIDAHVHLSGQSSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGSNAREFRYMVENGLTPAQALLAGTANAARLLGTFDRVGTLEAGKLADVVAVPGNPIADITVTERPLFVMKDGVVVRRDAVR

SEQ ID NO: 326 - protein, 420 aa, synthetic construct
  REGION 1..420: note = exemplary polypeptide
APSTPHTVVLRAARMFDGASGKIVEHPQLTIVGNKIVRIAQGKEEDNRTGIPAGADVIDLGDATLLPGFIDAHVHLSGQSSDNWYGDFFDNMMRFPAEQALYGAYYARITLEAGFTSVRDVGSTDWIALGLRNAIDAGKVPGPRMLVSNYAVGSTGGHADQDPIPPQKLAPAGPLQGVCNGPEECRAAVRYQIKYGANVIKFMPSGGVLSLSDPVDAPELSQDEMNAVIGEAHAWGRKVAAHAHGDKAAKMAVTAGVDSIEHGSFLEDDTLRMMKAKGVYLVPTISAIAHVEEKIATFPSVIAEKGRAAASHSRQMFRHAVQIGVPIALGTDAGVGAHGTNAREFRYMVDNGLTPAQSLMAGTANAARLLGTFDRVGTLEAGKLADVVAVPGNPIADITVTERPVFVMKDGVVVRRGAVR

SEQ ID NO: 327 - protein, 423 aa, synthetic construct
  REGION 1..423: note = exemplary polypeptide
ADAAPSTPHTVVLRAARMFDGASGKIVEHPQLTIVGNKIVRIAQGKEEDNRTGIPAGADVIDLGDATLLPGFIDAHVHLSGQSSDNWYGDFFDNMMRFPAEQALYGAYYARITLEAGFTSVRDVGSTDWIALGLRNAIDAGKVPGPRMLVSNYAVGSTGGHADQDPIPPQKLAPAGPLQGVCNGPEECRAAVRYQIKYGANVIKFMPSGGVLSLSDPVDAPELSQDEMNAVIGEAHAWGRKVAAHAHGDKAAKMAVTAGVDSIEHGSFLEDDTLRMMKAKGVYLVPTISAIAHVEEKIATFPSVIAEKGRAAASHSRQMFRHAVQIGVPIALGTDAGVGAHGTNAREFRYMVDNGLTPAQSLMAGTANAARLLGTFDRVGTLEAGKLADVVAVPGNPIADITVTERPVFVMKDGVVVRRGAVR

SEQ ID NO: 328 - protein, 416 aa, synthetic construct
  REGION 1..416: note = exemplary polypeptide
MPSTPHTVVLRAARMFDGASGKLVEHPQLTIVGNKIVRVAQDDKAAVPAGADVIDLGDATLLPGFIDAHVHLSGQSSDNWYGDFFDNMMRFPAEQALYGAWYARITLEAGFTSVRDVGSTDWIALGLRNAIDAGKVPGPRMLVSNYAIGSTGGHADQDPIPPQKLAPAGPLQGVCNGPDECRAAVRYQIKYGANVIKFMPSGGVLSLSDPVDAPELTQEEMNAVVGEAHAWGRKVAAHAHGDKAAKMAVAAGVDSIEHGSFLEDDTLRMMKAKGVYLVPTISAVMHVEEKIATFPPVIAEKGRAAAAHVRQTFRHAVQIGVPIALGTDAGVGAHGTNAREFRYMVDNGMAPAQALLAGTSNAARLLGTIDRVGTLEAGKLADVVAVPGNPVADITVTERPVFVMKDGVVVRRGAVR

SEQ ID NO: 329 - protein, 419 aa, synthetic construct
  REGION 1..419: note = exemplary polypeptide
ADAMPSTPHTVVLRAARMFDGASGKLVEHPQLTIVGNKIVRVAQDDKAAVPAGADVIDLGDATLLPGFIDAHVHLSGQSSDNWYGDFFDNMMRFPAEQALYGAWYARITLEAGFTSVRDVGSTDWIALGLRNAIDAGKVPGPRMLVSNYAIGSTGGHADQDPIPPQKLAPAGPLQGVCNGPDECRAAVRYQIKYGANVIKFMPSGGVLSLSDPVDAPELTQEEMNAVVGEAHAWGRKVAAHAHGDKAAKMAVAAGVDSIEHGSFLEDDTLRMMKAKGVYLVPTISAVMHVEEKIATFPPVIAEKGRAAAAHVRQTFRHAVQIGVPIALGTDAGVGAHGTNAREFRYMVDNGMAPAQALLAGTSNAARLLGTIDRVGTLEAGKLADVVAVPGNPVADITVTERPVFVMKDGVVVRRGAVR

SEQ ID NO: 330 - protein, 416 aa, synthetic construct
  REGION 1..416: note = exemplary polypeptide
APAASRTIVLRAARMFDGASGKLVEHPQLTIVGNKIVRIAQSDNAEVPASAEVIDLGDATLLPGFIDAHVHLSGQSSDNWYGDFFDNMMRFPAEQALYGAYYACITLEAGFTSVRDLGSTDWVALGLRNAIDAGKVPGPRMLVSNYAVGSTGGHADQDPIPPHRLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIATFPPVIAEKGRAAAAHSRQTFRHAVQIGVPIALGTDAGVGAHGTNAREFRYMVENGLTPVQSLLAGTANAARLLGIIDRVGTLEAGKLADIVAVPGNPVADITVTERPVFVMKDGVVVRRGAVR

SEQ ID NO: 331 - protein, 419 aa, synthetic construct
  REGION 1..419: note = exemplary polypeptide
ADAAPAASRTIVLRAARMFDGASGKLVEHPQLTIVGNKIVRIAQSDNAEVPASAEVIDLGDATLLPGFIDAHVHLSGQSSDNWYGDFFDNMMRFPAEQALYGAYYACITLEAGFTSVRDLGSTDWVALGLRNAIDAGKVPGPRMLVSNYAVGSTGGHADQDPIPPHRLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIATFPPVIAEKGRAAAAHSRQTFRHAVQIGVPIALGTDAGVGAHGTNAREFRYMVENGLTPVQSLLAGTANAARLLGIIDRVGTLEAGKLADIVAVPGNPVADITVTERPVFVMKDGVVVRRGAVR

SEQ ID NO: 332 - protein, 416 aa, synthetic construct
  REGION 1..416: note = exemplary polypeptide
APATPHTVVLRAARMFDGASGKLVERPQLTIVGNKIVRIAQADKAEVPAGAEVIDLGDSTLLPGFIDAHVHLSGQGSDNWYGDFFDNMMRFPAEQALYGAYYARITLEAGFTSVRDLGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGTNAREFRYMVENGLTPAQSLLAGTSNAARLLGTIDKVGTLEAGKLADVVAVPGNPIADITVTERPVFVMKDGVVVRRGAVR

SEQ ID NO: 333 - protein, 419 aa, synthetic construct
  REGION 1..419: note = exemplary polypeptide
ADAAPATPHTVVLRAARMFDGASGKLVERPQLTIVGNKIVRIAQADKAEVPAGAEVIDLGDSTLLPGFIDAHVHLSGQGSDNWYGDFFDNMMRFPAEQALYGAYYARITLEAGFTSVRDLGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKNVYLVPTISAIAHVEEKIGTFPPVIAEKGRAAAAHLRQTFRHAVQIGVPIALGTDAGVGAHGTNAREFRYMVENGLTPAQSLLAGTSNAARLLGTIDKVGTLEAGKLADVVAVPGNPIADITVTERPVFVMKDGVVVRRGAVR

SEQ ID NO: 334 - protein, 416 aa, synthetic construct
  REGION 1..416: note = exemplary polypeptide
TPSTPHTVVLRAARMFDGASGKLVERPQLTIVGNKIVRIAQADKADVPAGAEVIDLGDATLLPGFIDAHVHLSGQGSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKGVYLVPTISAIAHVEEKIATFPPVIAEKGRAAAAHLRQTFRHAVQIGVPIALGTDAGVGAHGTNAREFRYMVENGLSPAQSLLAGTSNAARLLGTIDRVGTLEAGKLADVVAVPGNPIADITVTERPVFVMKDGVVVRRGAVR

SEQ ID NO: 335 - protein, 419 aa, synthetic construct
  REGION 1..419: note = exemplary polypeptide
ADATPSTPHTVVLRAARMFDGASGKLVERPQLTIVGNKIVRIAQADKADVPAGAEVIDLGDATLLPGFIDAHVHLSGQGSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAHGDKAAKMAVAAGVDSIEHGSFIEDDTLRAMKAKGVYLVPTISAIAHVEEKIATFPPVIAEKGRAAAAHLRQTFRHAVQIGVPIALGTDAGVGAHGTNAREFRYMVENGLSPAQSLLAGTSNAARLLGTIDRVGTLEAGKLADVVAVPGNPIADITVTERPVFVMKDGVVVRRGAVR

SEQ ID NO: 336 - protein, 412 aa, synthetic construct
  REGION 1..412: note = exemplary polypeptide
VPAPPRPAVLKAARIFDSVSGHLEEPGLVVVAGAKIQAVGSDARIPDNAQVIDLGDATLMPGFIDAHVHLSSEMSDKWYEDWYKSMVRFPAEQALYGAHYAKATLEAGITTVRDLGSSDYISLGLRNAIKAGQIPGPRMLVANYAIGSTGGHADQDPVPPQRLAPAGPIQGVCNGPDECRAAVRYQLKYGADVIKFMPSGGVLSLSDPVDTPELTQEEMNAIVSEAHAWKRKVAAHCHGDRAAKMAIAAGVDSIEHGTFLQDDTLQEMKDKHIYLVPTLSAGYWVGLKADTYPPVIAEKARAASRQMQKMFQRAVKIGVPIAMGTDAGVEPHGQNAREFSLMTTNGLPPAQALLAATAKGADLLGISDQTGTLQPGKYADIVAVGGNPLLDIKATEHPLLVMKEGVIYVGGK

SEQ ID NO: 337 - protein, 489 aa, synthetic construct
  REGION 1..489: note = exemplary polypeptide
MKRPPRCQLNPRQNSRVEFPPSLCHYRAMLGETKNQFSLTPAPRSTRGLTKPQFPLRGCSLALAWTIFSTAGLAEEVVPAPPRPAVLKAARIFDSVSGHLEEPGLVVVAGAKIQAVGSDARIPDNAQVIDLGDATLMPGFIDAHVHLSSEMSDKWYEDWYKSMVRFPAEQALYGAHYAKATLEAGITTVRDLGSSDYISLGLRNAIKAGQIPGPRMLVANYAIGSTGGHADQDPVPPQRLAPAGPIQGVCNGPDECRAAVRYQLKYGADVIKFMPSGGVLSLSDPVDTPELTQEEMNAIVSEAHAWKRKVAAHCHGDRAAKMAIAAGVDSIEHGTFLQDDTLQEMKDKHIYLVPTLSAGYWVGLKADTYPPVIAEKARAASRQMQKMFQRAVKIGVPIAMGTDAGVEPHGQNAREFSLMTTNGLPPAQALLAATAKGADLLGISDQTGTLQPGKYADIVAVGGNPLLDIKATEHPLLVMKEGVIYVGGK

SEQ ID NO: 338 - protein, 489 aa, synthetic construct
  REGION 1..489: note = exemplary polypeptide
MKRPPRCQLNPRQNSRVEFPPSLCHYRAMLGETKNQFSLTPAPRSTRGLTKPQFPLRGCSLALAWTIFSTAGLAEEVVPAPPRPAVLKAARIFDSVSGHLEEPGLVVVAGAKIQAVGSDARIPDNAQVIDLGDATLMPGFIDAHVHLVSEMSDKWYEDWYKSMVRFPAEQALYGAHYAKATLEAGITTVRDLGEADYISLGLRNAIKAGQIPGPRMLVANYPIGSTGGHADQDPIPPQRLAPAGPIQGVCNGPDECRAAVRYQLKYGADVIKFMPSGGVLSLSDPVDTPELTQEEMNAIVSEAHAWKRKVAAHCHGDRAAKMAIAAGVDSIEHGTFLQDDTLQEMKDKHIYLVPTLSAVYWVGLKADTFPPVIAEKARAASRQLQKMFQRAVKIGVPIAMGTDAGLEPHGQNAREFSLMTTNGLPPAQALLAATAKGADLLGISDQTGTLQPGKYADIVAVGGNPLLDIKATEHPLLVMKEGVIYVGGK

SEQ ID NO: 339 - protein, 489 aa, synthetic construct
  REGION 1..489: note = exemplary polypeptide
MKRPPRCQLNPRQNSRVEFPPSLCHYRAMLGETKNQFSLTPAPRSTRGLTKPQFPLRGCSLALAWTIFSTAGLAEEVVPAPPRPAVLKAARIFDSVSGHLEEPGLVVVAGAKIQAVGSDARIPDNAQVIDLGDATLMPGFIDAHVHLSSEMSDKWYEDWYKSMVRFPAEQALYGAHYAKATLEAGITTVRDLGSSDYISLGLRNAIKAGQIPGPRMLVANYPIGSTGGHADQDPIPPQRLAPAGPIQGVCNGPDECRAAVRYQLKYGADVIKFMPSGGVISLSDPVDTPELTQEEMNAIVSEAHAWKRKVAAHCHGDRAAKMAIAAGVDSIEHGTFLQDDTLQEMKDKHIYLVPTLSAGYWVGLKADTFPPVIAEKARAVVRQIQKMFQRAVKIGVPIAMGTDAAVPPHGQNAREFSLMTTNGLPPAQALLAATAKGADLLGISDQTGTLQPGKYADIVAVGGNPLLDIKATEHPLLVMKEGVIYVGGK

SEQ ID NO: 340 - protein, 489 aa, synthetic construct
  REGION 1..489: note = exemplary polypeptide
MKRPPRCQLNPRQNSRVEFPPSLCHYRAMLGETKNQFSLTPAPRSTRGLTKPQFPLRGCSLALAWTIFSTAGLAEEVVPAPPRPAVLKAARIFDSVSGHLEEPGLVVVAGAKIQAVGSDARIPDNAQVIDLGDATLMPGFIDAHVHLSSEMSDKWYEDWYKSMVRFPAEQALYGAHYAKATLEAGITTVRDLGSSDYISLGLRNAIKAGQIPGPRMLVANYPIGSTGGHADQDPIPPQRLAPAGPIQGVCNGPDECRAAVRYQLKYGADVIKFMPSGGVISLSDPVDTPELTQEEMNAIVSEAHAWKRKVAAHCHGDRAAKMAIAAGVDSIEHGTFLQDDTLQEMKDKHIYLVPTLSAVYWVGLKADTFPPVIAEKARAVLRQMQKMFQRAVKIGVPIAMGTDAAVFPHGQNAREFSLMTTNGLPPAQALLAATAKGADLLGISDQTGTLQPGKYADIVAVGGNPLLDIKATEHPLLVMKEGVIYVGGK

SEQ ID NO: 341 - protein, 489 aa, synthetic construct
  REGION 1..489: note = exemplary polypeptide
MKRPPRCQLNPRQNSRVEFPPSLCHYRAMLGETKNQFSLTPAPRSTRGLTKPQFPLRGCSLALAWTIFSTAGLAEEVVPAPPEPAVLKAARIFDSVSGHLEENGLVVVAGAKIQAVGSDARIPDNAQVIDLGDATLMPGFIDAHVHLVVEMSDDYYEDWYKSMVRFPAEHALYGAHYAKAELEAGITTVRDLASRDYISLGLRNAIKAGQIPGPRMLVANFPIGSTGGHADQDPIPPNKLAPAGPIQGVCNGPDECRAAVRYQLKYGADVIKFCPSGGVLTLSDPVDTPQLTQEEMNAIVSEAHAWKRKVAAHCHGDRAAKMAIAAGVDSIEHGSFLQDDTLQEMKDKHIYLVPTLSAVYWVGLKADTYPPIIAEKARAASRQMQKMFQRAVKIGVPIAMGTDAGVEPHGQNAREFSLMTTNGLPPAQALLAATAKGADLLGISDQTGTLQPGKYADIVAVGGNPLLDIKATEHPLLVMKEGVIYVGGK

SEQ ID NO: 342 - protein, 489 aa, synthetic construct
  REGION 1..489: note = exemplary polypeptide
MKRPPRCQLNPRQNSRVEFPPSLCHYRAMLGETKNQFSLTPAPRSTRGLTKPQFPLRGCSLALAWTIFSTAGLAEEVVPAPPRPAVLKAARIFDSVSGHLEEPGLVVVAGAKIQAVGSDARIPDNAQVIDLGDATLMPGFIDAHVHLSSEMSDKYYEDWYKSMVRFPAEHALYGAHYAKAELEAGITTVRDLASRDYISLGLRNAIKAGQIPGPRMLVANFPIGSTGGHADQDPIPPNKLAPAGPIQGVCNGPDECRAAVRYQLKYGADVIKFCPSGGVLTLSDPVDTPQLTQEEMNAIVSEAHAWKRKVAAHCHGDRAAKMAIAAGVDSIEHGSFLQDDTLQEMKDKHIYLVPTLSAVYWVGLKADTYPPIIAEKARAASRQIQKMFQEAVKIGVPIAMGTDSGLPPHGQNAREFSLMTTNGLPPAQALLAATAKGADLLGISDQTGTLQPGKYADIVAVGGNPLLDIKATEHPLLVMKEGVIYVGGK

SEQ ID NO: 343 - protein, 489 aa, synthetic construct
  REGION 1..489: note = exemplary polypeptide
MKRPPRCQLNPRQNSRVEFPPSLCHYRAMLGETKNQFSLTPAPRSTRGLTKPQFPLRGCSLALAWTIFSTAGLAEEVVPAPPEPAVLKAARIFDSVSGHLEENGLVVVAGAKIQAVGSDARIPDNAQVIDLGDATLMPGFIDAHVHLVVEMSDDYYEDWYKSMVRFPAEHALYGAHYAKAELEAGITTVRDLASRDYISLGLRNAIKAGQIPGPRMLVANFPIGSTGGHADQDPIPPNKLAPAGPIQGVCNGPDECRAAVRYQLKYGADVIKFCPSGGVITLSDPVDTPQLTQEEMNAIVSEAHAWKRKVAAHCHGDRAAKMAIAAGVDSIEHGSFLQDDTLQEMKDKHIYLVPTLSAVYWVGLKADTFPPIIAEKARAAVRQIQKMFQEAVKIGVPIAMGTDSGLPPHGQNAREFSLMTTNGLPPAQALLAATAKGADLLGISDQTGTLQPGKYADIVAVGGNPLLDIKATEHPLLVMKEGVIYVGGK

SEQ ID NO: 344 - protein, 489 aa, synthetic construct
  REGION 1..489: note = exemplary polypeptide
MKRPPRCQLNPRQNSRVEFPPSLCHYRAMLGETKNQFSLTPAPRSTRGLTKPQFPLRGCSLALAWTIFSTAGLAEEVVPAPPRPAVLKAARIFDSVSGHLEENGLVVVAGAKIQRVGSDARIPDNAQVIDLGDATLMPGFIDAHVHLVSEMSDKYYEDWYKSMVRFPAEHALYGAYYAKAELEAGITTVRDLASRDYISLGLRNAIKAGQIPGPRMLVANFPIGSTGGHADQDPIPPNKLAPAGPIQGVCNGPDECRAAVRYQLKYGADVIKFCPSGGVITLSDPVDTPQLTQDEMNAIVSEAHAWKRKVAAHCHGDRAAKMAIAAGVDSIEHGSFLQDDTLQEMKDKHIYLVPTLSAVYWVGLKADTFPPIIAEKARAAVRQIQKMFQEAVKIGVPIAMGTDSGLPPHGQNAREFSLMTTNGLPPAQALLAATAKGADLLGISDQTGTLQPGKYADIVAVGGNPLLDIKATEHPLLVMKEGVIYVGGK

SEQ ID NO: 345 - protein, 489 aa, synthetic construct
  REGION 1..489: note = exemplary polypeptide
MKRPPRCQLNPRQNSRVEFPPSLCHYRAMLGETKNQFSLTPAPRSTRGLTKPQFPLRGCSLALAWTIFSTAGLAEEVVPAPPEPAVLKCARIFDSVSGHLEENGLVVVAGAKIQAVGSDARIPDNAQVIDLGDATCMPGFIDAHVHLVVEMSDQYRPRAFDDWQRFPAEHALRGAHWAKAELEAGITTVRDLAERDYISLGLRNAIKAGQIPGPRMLVANFPIGSTSGHCDMDSIPPNKLAPVGPIQGTCNGPDECRAAVRYQLKYGADVIKFCPSSGAITFSNPVDTPELTQEEMNAIVSEAHAWKRKVAAHCHGDRAAKMAIAAGVDSIEHGTFLQDDTLQEMKDKHIYLVPTLSAGYWVGLKADTYPPVIAEKARAASRQMQKMFQRAVKIGVPIAMGTDAGVEPHGQNAREFSLMTTNGLPPAQALLAATAKGADLLGISDQTGTLQPGKYADIVAVGGNPLLDIKATEHPLLVMKEGVIYVGGK

SEQ ID NO: 346 - protein, 489 aa, synthetic construct
  REGION 1..489: note = exemplary polypeptide
MKRPPRCQLNPRQNSRVEFPPSLCHYRAMLGETKNQFSLTPAPRSTRGLTKPQFPLRGCSLALAWTIFSTAGLAEEVVPAPPRPAVLKAARIFDSVSGHLEEPGLVVVAGAKIQAVGSDARIPDNAQVIDLGDATLMPGFIDAHVHLSSEMSDKWYEDWYKSMVRFPAEQALYGAHYAKATLEAGITTVRDLGSSDYISLGLRNAIKAGQIPGPRMLVANYAIGSTGGHADQDPVPPQRLAPAGPIQGTCNGPDECRAAVRYQLKYGADVIKFCPSSGAITFSNPADTPQLTPEEMNAIVSEAEAWKRYVAAHCQGDRAAKMAIAAGVRSIEHGSFLEDDTLQEMKDKHICLVPTLSGVCEAGAQADCFPPVMAEKARAGMRSLQKMFQEAVKIGVPIAMGTDSALPANGQQAREFSLMTTNGLPPAQALLAATAKGADCLRISDQTGTLQPGKYADIVAVGGNPLLDIKATEHPLLVMKEGVIYVGGK

SEQ ID NO: 347 - protein, 489 aa, synthetic construct
  REGION 1..489: note = exemplary polypeptide
MKRPPRCQLNPRQNSRVEFPPSLCHYRAMLGETKNQFSLTPAPRSTRGLTKPQFPLRGCSLALAWTIFSTAGLAEEVVPAPPRPAVLKAARIFDSVSGHLEENGLVVVAGAKIQAVGSDARIPDNAQVIDLGDATCMPGFIDAHVHLVVEMSDQYRPRAFDDWQRFPAEHALRGAHWAKATLEAGITTVRDLGESDYISLGLRNAIKAGQIPGPRMLVANFAIGSTGGHADQDSVPPQKLAPVGPIQGTCNGPDECRAAVRYQLKYGADVIKFCPSSGAITFSNPVDTPELTQEEMNAIVSEAEAWKRYVAAHCQGDRAAKMAIAAGVRSIEHGSFLEDDTLQEMKDKHICLVPTLSGGYWVGLKADTYPPVIAEKARAASRQLQKMFQRAVKIGVPIAMGTDAGVEPHGQNAREFSLMTTNGLPPAQALLAATAKGADCLRISDQTGTLQPGKYADIVAVGGNPLLDIKATEHPLLVMKEGVIYVGGK

SEQ ID NO: 348 - protein, 412 aa, synthetic construct
  REGION 1..412: note = exemplary polypeptide
RAHIVEPVAIKAKRVFTGVTGAVIENGVVIVDDKGRIAAISRVVPKGAKVIDLGDATLLPGFIDAHVHLSGEMSDNWYKDFRDRMLRFPAEQAHYAARNAKKTLMAGFTTVRDVGSADWQASALRNAVNAGVIVGPRILASNYAIGATGGHGDGTPFPPDRVAPDGPLKGVCNGADQCRAAVRYQIKYGADVIKFMPSGGVLSLSDPVDVPELTQDEINAIISEAHAWHRKVAAHAHGDEAARMAVKAGVDSIEHGTFLTDETLKQMKKQGTYLVATLFAGEWVGMKADNYPPAVAEKARAAAAQMQRMFARAVKIGVPIAFGTDAGVEPHGLNAHEFSLMVKNGMSPKAALLAATREAAKLLGVIDRTGTLEPGKAADMVAVRGDPLADIKATEQVIFVMKGGKIYRNEVE

SEQ ID NO: 349 - protein, 438 aa, synthetic construct
  REGION 1..438: note = exemplary polypeptide
MKRFSFSAWIGALSLLLITGLGALTARAHIVEPVAIKAKRVFTGVTGAVIENGVVIVDDKGRIAAISRVVPKGAKVIDLGDATLLPGFIDAHVHLSGEMSDNWYKDFRDRMLRFPAEQAHYAARNAKKTLMAGFTTVRDVGSADWQASALRNAVNAGVIVGPRILASNYAIGATGGHGDGTPFPPDRVAPDGPLKGVCNGADQCRAAVRYQIKYGADVIKFMPSGGVLSLSDPVDVPELTQDEINAIISEAHAWHRKVAAHAHGDEAARMAVKAGVDSIEHGTFLTDETLKQMKKQGTYLVATLFAGEWVGMKADNYPPAVAEKARAAAAQMQRMFARAVKIGVPIAFGTDAGVEPHGLNAHEFSLMVKNGMSPKAALLAATREAAKLLGVIDRTGTLEPGKAADMVAVRGDPLADIKATEQVIFVMKGGKIYRNEVE

SEQ ID NO: 350 - protein, 438 aa, synthetic construct
  REGION 1..438: note = exemplary polypeptide
MKRFSFSAWIGALSLLLITGLGALTARAHIVEPVAIKAKRVFTGVTGAVIENGVVIVDDKGRIAAISRVVPKGAKVIDLGDATLLPGFIDAHVHLVEEMSDNWYKDFRDRMLRFPAEQAHYAARNAKKTLMAGFTTVRDVGSSDWQASALRNAVNAGVIVGPRILASNYPIGATGGHGDGTPIPPDRVAPDGPLKGVCNGADQCRAAVRYQIKYGADVIKFMPSGGVLSLSDPVDVPELTQDEINAIISEAHAWHRKVAAHAHGDEAARMAVKAGVDSIEHGTFLTDETLKQMKKQGTYLVATLFAVEWVGMKADNFPPAVAEKARAAAAQLQRMFARAVKIGVPIAFGTDAGLEPHGLNAHEFSLMVKNGMSPKAALLAATREAAKLLGVIDRTGTLEPGKAADMVAVRGDPLADIKATEQVIFVMKGGKIYRNEVE

SEQ ID NO: 351 - protein, 438 aa, synthetic construct
  REGION 1..438: note = exemplary polypeptide
MKRFSFSAWIGALSLLLITGLGALTARAHIVEPVAIKAKRVFTGVTGAVIENGVVIVDDKGRIAAISRVVPKGAKVIDLGDATLLPGFIDAHVHLSGEMSDNWYKDFRDRMLRFPAEQAHYAARNAKKTLMAGFTTVRDVGSADWQASALRNAVNAGVIVGPRILASNYPIGATGGHGDGTPIPPDRVAPDGPLKGVCNGADQCRAAVRYQIKYGADVIKFMPSGGVISLSDPVDVPELTQDEINAIISEAHAWHRKVAAHAHGDEAARMAVKAGVDSIEHGTFLTDETLKQMKKQGTYLVATLFAGEWVGMKADNFPPAVAEKARAVVAQIQRMFARAVKIGVPIAFGTDAAVPPHGLNAHEFSLMVKNGMSPKAALLAATREAAKLLGVIDRTGTLEPGKAADMVAVRGDPLADIKATEQVIFVMKGGKIYRNEVE

SEQ ID NO: 352 - protein, 438 aa, synthetic construct
  REGION 1..438: note = exemplary polypeptide
MKRFSFSAWIGALSLLLITGLGALTARAHIVEPVAIKAKRVFTGVTGAVIENGVVIVDDKGRIAAISRVVPKGAKVIDLGDATLLPGFIDAHVHLSGEMSDNWYKDFRDRMLRFPAEQAHYAARNAKKTLMAGFTTVRDVGSADWQASALRNAVNAGVIVGPRILASNYPIGATGGHGDGTPIPPDRVAPDGPLKGVCNGADQCRAAVRYQIKYGADVIKFMPSGGVISLSDPVDVPELTQDEINAIISEAHAWHRKVAAHAHGDEAARMAVKAGVDSIEHGTFLTDETLKQMKKQGTYLVATLFAVEWVGMKADNFPPAVAEKARAVLAQMQRMFARAVKIGVPIAFGTDAAVFPHGLNAHEFSLMVKNGMSPKAALLAATREAAKLLGVIDRTGTLEPGKAADMVAVRGDPLADIKATEQVIFVMKGGKIYRNEVE

SEQ ID NO: 353 - protein, 438 aa, synthetic construct
  REGION 1..438: note = exemplary polypeptide
MKRFSFSAWIGALSLLLITGLGALTARAHIVRPVAIKAKRVFTGVTGAVIEPGVVIVDDKGRIAAISRVVPKGAKVIDLGDATLLPGFIDAHVHLVVEMSDDYYKDFRDRMLRFPAEHAHYAARNAKKELMAGFTTVRDVASRDWQASALRNAVNAGVIVGPRILASNFPIGATGGHGDGTPIPPNKVAPDGPLKGVCNGADQCRAAVRYQIKYGADVIKFCPSGGVLTLSDPVDVPQLTQDEINAIISEAHAWHRKVAAHAHGDEAARMAVKAGVDSIEHGSFLTDETLKQMKKQGTYLVATLFAVEWVGMKADNYPPIVAEKARAAAAQMQRMFARAVKIGVPIAFGTDAGVEPHGLNAHEFSLMVKNGMSPKAALLAATREAAKLLGVIDRTGTLEPGKAADMVAVRGDPLADIKATEQVIFVMKGGKIYRNEVE

SEQ ID NO: 354 - protein, 438 aa, synthetic construct
  REGION 1..438: note = exemplary polypeptide
MKRFSFSAWIGALSLLLITGLGALTARAHIVEPVAIKAKRVFTGVTGAVIENGVVIVDDKGRIAAISRVVPKGAKVIDLGDATLLPGFIDAHVHLSGEMSDNYYKDFRDRMLRFPAEHAHYAARNAKKELMAGFTTVRDVASRDWQASALRNAVNAGVIVGPRILASNFPIGATGGHGDGTPIPPNKVAPDGPLKGVCNGADQCRAAVRYQIKYGADVIKFCPSGGVLTLSDPVDVPQLTQDEINAIISEAHAWHRKVAAHAHGDEAARMAVKAGVDSIEHGSFLTDETLKQMKKQGTYLVATLFAVEWVGMKADNYPPIVAEKARAAAAQIQRMFAEAVKIGVPIAFGTDSGLPPHGLNAHEFSLMVKNGMSPKAALLAATREAAKLLGVIDRTGTLEPGKAADMVAVRGDPLADIKATEQVIFVMKGGKIYRNEVE

SEQ ID NO: 355 - protein, 438 aa, synthetic construct
  REGION 1..438: note = exemplary polypeptide
MKRFSFSAWIGALSLLLITGLGALTARAHIVRPVAIKAKRVFTGVTGAVIEPGVVIVDDKGRIAAISRVVPKGAKVIDLGDATLLPGFIDAHVHLVVEMSDDYYKDFRDRMLRFPAEHAHYAARNAKKELMAGFTTVRDVASRDWQASALRNAVNAGVIVGPRILASNFPIGATGGHGDGTPIPPNKVAPDGPLKGVCNGADQCRAAVRYQIKYGADVIKFCPSGGVITLSDPVDVPQLTQDEINAIISEAHAWHRKVAAHAHGDEAARMAVKAGVDSIEHGSFLTDETLKQMKKQGTYLVATLFAVEWVGMKADNFPPIVAEKARAAVAQIQRMFAEAVKIGVPIAFGTDSGLPPHGLNAHEFSLMVKNGMSPKAALLAATREAAKLLGVIDRTGTLEPGKAADMVAVRGDPLADIKATEQVIFVMKGGKIYRNEVE

SEQ ID NO: 356 - protein, 438 aa, synthetic construct
  REGION 1..438: note = exemplary polypeptide
MKRFSFSAWIGALSLLLITGLGALTARAHIVEPVAIKAKRVFTGVTGAVIEPGVVIVDDKGRRAAISRVVPKGAKVIDLGDATLLPGFIDAHVHLVGEMSDNYYKDFRDRMLRFPAEHAHYAAYNAKKELMAGFTTVRDVASRDWQASALRNAVNAGVIVGPRILASNFPIGATGGHGDGTPIPPNKVAPDGPLKGVCNGADQCRAAVRYQIKYGADVIKFCPSGGVITLSDPVDVPQLTQEEINAIISEAHAWHRKVAAHAHGDEAARMAVKAGVDSIEHGSFLTDETLKQMKKQGTYLVATLFAVEWVGMKADNFPPIVAEKARAAVAQIQRMFAEAVKIGVPIAFGTDSGLPPHGLNAHEFSLMVKNGMSPKAALLAATREAAKLLGVIDRTGTLEPGKAADMVAVRGDPLADIKATEQVIFVMKGGKIYRNEVE

SEQ ID NO: 357 - protein, 438 aa, synthetic construct
  REGION 1..438: note = exemplary polypeptide
MKRFSFSAWIGALSLLLITGLGALTARAHIVRPVAIKCKRVFTGVTGAVIEPGVVIVDDKGRIAAISRVVPKGAKVIDLGDATCLPGFIDAHVHLVVEMSDQYRPRAFKDWQRFPAEHAHRAARWAKKELMAGFTTVRDVAERDWQASALRNAVNAGVIVGPRILASNFPIGATSGHCDMTSIPPNKVAPVGPLKGTCNGADQCRAAVRYQIKYGADVIKFCPSSGAITFSNPVDVPELTQDEINAIISEAHAWHRKVAAHAHGDEAARMAVKAGVDSIEHGTFLTDETLKQMKKQGTYLVATLFAGEWVGMKADNYPPAVAEKARAAAAQMQRMFARAVKIGVPIAFGTDAGVEPHGLNAHEFSLMVKNGMSPKAALLAATREAAKLLGVIDRTGTLEPGKAADMVAVRGDPLADIKATEQVIFVMKGGKIYRNEVE

SEQ ID NO: 358 - protein, 438 aa, synthetic construct
  REGION 1..438: note = exemplary polypeptide
MKRFSFSAWIGALSLLLITGLGALTARAHIVEPVAIKAKRVFTGVTGAVIENGVVIVDDKGRIAAISRVVPKGAKVIDLGDATLLPGFIDAHVHLSGEMSDNWYKDFRDRMLRFPAEQAHYAARNAKKTLMAGFTTVRDVGSADWQASALRNAVNAGVIVGPRILASNYAIGATGGHGDGTPFPPDRVAPDGPLKGTCNGADQCRAAVRYQIKYGADVIKFCPSSGAITFSNPADVPQLTPDEINAIISEAEAWHRYVAAHAQGDEAARMAVKAGVRSIEHGSFLEDETLKQMKKQGTCLVATLFGVCEAGAQADCFPPAMAEKARAGMASLQRMFAEAVKIGVPIAFGTDSALPANGLQAHEFSLMVKNGMSPKAALLAATREAAKCLRVIDRTGTLEPGKAADMVAVRGDPLADIKATEQVIFVMKGGKIYRNEVE

SEQ ID NO: 359 - protein, 438 aa, synthetic construct
  REGION 1..438: note = exemplary polypeptide
MKRFSFSAWIGALSLLLITGLGALTARAHIVEPVAIKAKRVFTGVTGAVIEPGVVIVDDKGRIAAISRVVPKGAKVIDLGDATCLPGFIDAHVHLVVEMSDQYRPRAFKDWQRFPAEHAHRAARWAKKTLMAGFTTVRDVGEADWQASALRNAVNAGVIVGPRILASNFAIGATGGHGDGTSFPPKRVAPVGPLKGTCNGADQCRAAVRYQIKYGADVIKFCPSSGAITFSNPVDVPELTQDEINAIISEAEAWHRYVAAHAQGDEAARMAVKAGVRSIEHGSFLEDETLKQMKKQGTCLVATLFGGEWVGMKADNYPPAVAEKARAAAAQLQRMFARAVKIGVPIAFGTDAGVEPHGLNAHEFSLMVKNGMSPKAALLAATREAAKCLRVIDRTGTLEPGKAADMVAVRGDPLADIKATEQVIFVMKGGKIYRNEVE

SEQ ID NO: 360 - protein, 7 aa, synthetic construct
  REGION 1..7: note = conservative motif
VMPGLID

SEQ ID NO: 361 - protein, 5 aa, synthetic construct
  REGION 1..5: note = conservative motif
KGGSL

SEQ ID NO: 362 - protein, 13 aa, synthetic construct
  REGION 1..13: note = conservative motif
LLLAGFTTVRDCG

SEQ ID NO: 363 - protein, 7 aa, synthetic construct
  REGION 1..7: note = conservative motif
QTFGHGE

SEQ ID NO: 364 - protein, 11 aa, synthetic construct
  REGION 1..11: note = conservative motif
FATGGVLSQRD

SEQ ID NO: 365 - protein, 5 aa, synthetic construct
  REGION 1..5: note = conservative motif
IVNEA

SEQ ID NO: 366 - protein, 8 aa, synthetic construct
  REGION 1..8: note = conservative motif
IGVDEWGL

SEQ ID NO: 367 - protein, 6 aa, synthetic construct
  REGION 1..6: note = conservative motif
GFETGL

SEQ ID NO: 368 - protein, 440 aa, synthetic construct
  REGION 1..440: note = exemplary polypeptide
MTTPTRILTAVLSLLAANACAADAAPATPHTVVLRAARMFDGASGKLVERPQLTIVGNKIVRIAQADKADVPAGAEVIDLGDATLLPGFIDAHVHLSGQGSDNWYGDFFDNMMRFPAEQALYGAYYARITLEAGFTSVRDVGSTDWVALGLRNAIDAGKVPGPRMLVSNYAVGSTGGHADQDPIPPHKLPLAGPLQGVCNGPEECRAAVRYQIKYGANVIKFMPSGGVLSLSDPVDAPELSQDEMNAVIGESHAWGRKVAAHAPGDKAAKMAVAAGVDSIEHGSFIEDDTLRAMKAKGVYLVPTISAIAHVEEKIATFPPVIAEKGRAAAAHLRQTFRHAVQIGVPIALGTDAGVGAHGTNAREFRYMVENGLSPAQSLLAGTSNAARLLGTIDRVGTLEAGKLADVVAVPGNPIADITVTERPVFVMKDGVVVRRGAVR

SEQ ID NO: 369 - protein, 440 aa, synthetic construct
  REGION 1..440: note = exemplary polypeptide
MRTRTRILTASLALLAANAWAADAMPSTPHTVVLRAARMFDGASGKLVEHPQLTIVGNKIVRVAQDDKAAVPAGADVIDLGDATLLPGFIDAHVHLSGQSSDNWYGDFFDNMMRFPAEQALYGAWYARITLEAGFTSVRDVGSTDWIALGLRNAIDAGKVPGPRMLVSNYAIGSTGGHADQDPIPPQKLAPAGPLQGVCNGPDECRAAVRYQIKYGANVIKFMPSGGVLSLSDPVDAPELTQEEMNAVVGEAHAWGRKVAAHAHGDKAAKMAVAAGVDSIEHGSFLEDDTLRMMKAKGVYLVPTISAVMHVEEKIATFPPVIAEKGRAAAAHVRQTFRHAVQIGVPIALGTDAGVGAHGTNAREFRYMVDNGMAPAQALLAGTSNAARLLGTIDRVGTLEAGKLADVVAVPGNPVADITVTERPVFVMKDGVVVRRGAVR

SEQ ID NO: 370 - protein, 398 aa, synthetic construct
  REGION 1..398: note = exemplary polypeptide
MFDSVSGRLIDNAQVLVVGNKIARVGANLSAPAGAETIDLGDATLLPGFIDAHVHLSGQSSDNWYGDFFDNMMRFPAEQALYGAYNARITLEAGFTSVRDVGSTDWIALGLRNAIDAGKIPGPRMLVSNYAVGSTGGHADQDPVPPQKIAVAGPIQGVCNGPDECRAAVRYQIKYGANVIKFMPSGGVLSLSDPVDAPELTQDEMNAIIGEAHAWGRKVAAHAHGDKAAKMAVQAGVDSIEHGSFLEDDTLRLMKQKGVFLVPTISAVAHVEEKIATFPPVIADKGRAAANHLRTTFRHAAQIGVPIALGTDAAVGPHGANAREFNFMVQNGLTPAQALMAGTCNGARLLGTIDKVGTLQEGKLADIVAVPGNPLTDIQATERPLFVMKDGVVVKRAR